(19) United States
(12) Patent Application Publication
(10) Pub. No.: US 2025/0388628 A1
(43) Pub. Date: Dec. 25, 2025

(54) ENGINEERED ADENO-ASSOCIATED VIRUS CAPSIDS

(71) Applicant: Genentech, Inc., South San Francisco, CA (US)

(72) Inventors: Arvind RAJPAL, San Francisco, CA (US); Travis William BAINBRIDGE, Woodside, CA (US); Beyza BULUTOGLU BAYKARA, Foster City, CA (US); Yoon Min KIM, San Francisco, CA (US)

(21) Appl. No.: 19/220,420

(22) Filed: May 28, 2025

Related U.S. Application Data
(63) Continuation of application No. PCT/US2023/081799, filed on Nov. 30, 2023.
(60) Provisional application No. 63/385,488, filed on Nov. 30, 2022.

(51) Int. Cl. C07K 14/005; C12N 15/86

(52) U.S. Cl. CPC C07K 14/005; C12N 15/86; C12N 2750/14122; C12N 2750/14143

(57) ABSTRACT

Provided herein are engineered adeno-associated virus (AAV) capsids having altered (e.g., increased) affinity for AAV receptor (AAVR); methods of using the same; and methods of determining the affinity of an AAV capsid for a query protein using virus-like particles (VLPs) having a capsid consisting of the VP3 subunit of the AAV capsid.

### CROSS-REFERENCE TO RELATED APPLICATIONS

This application is a continuation of International Patent Application No. PCT/US2023/081799, filed on Nov. 30, 2023, which claims benefit to U.S. Provisional Application No. 63/385,488, filed on Nov. 30, 2022, the entire contents of which are incorporated herein by reference in their entirety.

### SEQUENCE LISTING

The instant application contains a Sequence Listing which has been submitted electronically in XML format and is hereby incorporated by reference in its entirety. Said XML copy, created on May 22, 2025, is named 50474-282003_Sequence_Listing_5_22_25 and is 21,364 bytes in size.

### FIELD OF THE INVENTION

Provided herein are engineered adeno-associated virus (AAV) capsids having increased affinity for AAV receptor (AAVR); methods of using the same; and methods of determining the affinity of an AAV capsid for a query protein using virus-like particles (VLPs) having a capsid consisting of the VP3 subunit of the AAV capsid.

### BACKGROUND

Adeno-associated viruses (AAVs) are non-pathogenic single-stranded DNA (ssDNA) viruses that may be used, e.g., as vectors for gene therapy. AAVs have a non-enveloped icosahedral capsid composed of major capsid protein VP1, minor capsid protein VP2, and minor capsid protein VP3, which are encoded by overlapping genes.

An important factor in the use of AAVs is mitigation of their immunogenicity in the subject. Immunogenicity has been shown to correlate with viral genome dose.

The AAV receptor (AAVR) is critical for transduction of most AAV serotypes into the cell. Improving the affinity of the AAV capsid to AAVR may increase transduction efficiency, thus reducing dose requirements and mitigating immunogenicity.

Therefore, the development of AAV capsids having altered (e.g., increased) affinity for AAVR represents an important unmet need.

### SUMMARY OF THE INVENTION

In one aspect, the disclosure features a method of improving transduction efficiency of an adeno-associated virus (AAV), the method comprising providing a modified AAV having a capsid comprising (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation; wherein the amino acid substitution mutations are numbered relative to the AAV9 major capsid protein VP1 (SEQ ID NO: 1).

In another aspect, the disclosure features a method of improving transduction efficiency of an adeno-associated virus (AAV), the method comprising contacting a target cell with a modified AAV having a capsid comprising (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation; wherein the amino acid substitution mutations are numbered relative to the AAV9 major capsid protein VP1 (SEQ ID NO: 1), thereby improving transduction efficiency.

In some aspects, (a) the major capsid protein VP1 further comprises an A273N amino acid substitution mutation; (b) the minor capsid protein VP2 further comprises an A273N amino acid substitution mutation; and/or (c) the minor capsid protein VP3 further comprises an A273N amino acid substitution mutation.

In some aspects, (a) the major capsid protein VP1 further comprises a Q387K amino acid substitution mutation; (b) the minor capsid protein VP2 further comprises a Q387K amino acid substitution mutation; and/or (c) the minor capsid protein VP3 further comprises a Q387K amino acid substitution mutation.

In some aspects, (a) the major capsid protein VP1 further comprises an E500R amino acid substitution mutation; (b) the minor capsid protein VP2 further comprises an E500R amino acid substitution mutation; and/or (c) the minor capsid protein VP3 further comprises an E500R amino acid substitution mutation.

In some aspects, (a) the major capsid protein VP1 further comprises an E500P amino acid substitution mutation; (b) the minor capsid protein VP2 further comprises an E500P amino acid substitution mutation; and/or (c) the minor capsid protein VP3 further comprises an E500P amino acid substitution mutation.

In some aspects, (a) the major capsid protein VP1 further comprises A273N, Q387K, and E500R amino acid substitution mutations; (b) the minor capsid protein VP2 further comprises A273N, Q387K, and E500R amino acid substitution mutations; and/or (c) the minor capsid protein VP3 further comprises A273N, Q387K, and E500R amino acid substitution mutations.

In another aspect, the disclosure features a method of improving transduction efficiency of an AAV, the method comprising providing a modified AAV having a capsid comprising (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; wherein the amino acid substitution mutations are numbered relative to the AAV9 major capsid protein VP1 (SEQ ID NO: 1).

In another aspect, the disclosure features a method of improving transduction efficiency of an AAV, the method comprising contacting a target cell with a modified AAV having a capsid comprising (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; wherein the amino acid substitution mutations are numbered relative to the AAV9 major capsid protein VP1 (SEQ ID NO: 1), thereby improving transduction efficiency.

In another aspect, the disclosure features a method of improving transduction efficiency of an AAV, the method comprising providing a modified AAV having a capsid comprising (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations; wherein the amino acid substitution mutations are numbered relative to the AAV9 major capsid protein VP1 (SEQ ID NO: 1).

In some aspects, the method comprises contacting a target cell with the modified AAV, thereby improving transduction efficiency.

In some aspects, the transduction efficiency is improved relative to the transduction efficiency of an AAV having a capsid not comprising any of the indicated amino acid substitution mutations.

In another aspect, the disclosure features a method of delivering a cargo to a cell, the method comprising contacting a target cell with a modified AAV having a capsid comprising (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation; wherein the cargo is encapsulated by the AAV; wherein the amino acid substitution mutations are numbered relative to the AAV9 major capsid protein VP1 (SEQ ID NO: 1); and wherein the AAV has an increased rate of transduction into a target cell relative to an AAV having a capsid not comprising any of the indicated amino acid substitution mutations.

In some aspects, (a) the major capsid protein VP1 further comprises an A273N amino acid substitution mutation; (b) the minor capsid protein VP2 further comprises an A273N amino acid substitution mutation; and/or (c) the minor capsid protein VP3 further comprises an A273N amino acid substitution mutation.

In some aspects, (a) the major capsid protein VP1 further comprises a Q387K amino acid substitution mutation; (b) the minor capsid protein VP2 further comprises a Q387K amino acid substitution mutation; and/or (c) the minor capsid protein VP3 further comprises a Q387K amino acid substitution mutation.

In some aspects, (a) the major capsid protein VP1 further comprises an E500R amino acid substitution mutation; (b) the minor capsid protein VP2 further comprises an E500R amino acid substitution mutation; and/or (c) the minor capsid protein VP3 further comprises an E500R amino acid substitution mutation.

In some aspects, (a) the major capsid protein VP1 further comprises an E500P amino acid substitution mutation; (b) the minor capsid protein VP2 further comprises an E500P amino acid substitution mutation; and/or (c) the minor capsid protein VP3 further comprises an E500P amino acid substitution mutation.

In some aspects, (a) the major capsid protein VP1 further comprises A273N, Q387K, and E500R amino acid substitution mutations; (b) the minor capsid protein VP2 further comprises A273N, Q387K, and E500R amino acid substitution mutations; and/or (c) the minor capsid protein VP3 further comprises A273N, Q387K, and E500R amino acid substitution mutations.

In another aspect, the disclosure features a method of delivering a cargo to a cell, the method comprising contacting a target cell with a modified AAV having a capsid comprising (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; wherein the cargo is encapsulated by the AAV; wherein the amino acid substitution mutations are numbered relative to the AAV9 major capsid protein VP1 (SEQ ID NO: 1); and wherein the AAV has an increased rate of transduction into a target cell relative to an AAV9 having a capsid not comprising any of the indicated amino acid substitution mutations.

In another aspect, the disclosure features a method of delivering a cargo to a cell, the method comprising contacting a target cell with a modified AAV having a capsid comprising (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations; wherein the cargo is encapsulated by the AAV; wherein the amino acid substitution mutations are numbered relative to the AAV9 major capsid protein VP1 (SEQ ID NO: 1); and wherein the AAV has an increased rate of transduction into a target cell relative to an AAV9 having a capsid not comprising any of the indicated amino acid substitution mutations.

In some aspects, the modified AAV has an increased affinity for the AAV receptor (AAVR) relative to an AAV having a capsid that does not comprise any of the indicated amino acid substitution mutations.

In some aspects, the modified AAV has an increased rate of delivery to the nucleus of the target cell relative to an AAV having a capsid that does not comprise any of the indicated amino acid substitution mutations.

In some aspects, the modified AAV transduces the target cell at a lower dose than an AAV having a capsid that does not comprise any of the indicated amino acid substitution mutations.

In another aspect, the disclosure features a method of improving the affinity of an AAV for AAV receptor (AAVR), the method comprising providing a modified AAV having a capsid comprising: (i) (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation; (ii) (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; or (iii) (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations; wherein the amino acid substitution mutations are numbered relative to the AAV9 major capsid protein VP1 (SEQ ID NO: 1).

In another aspect, the disclosure features a method of increasing the rate of delivery of an AAV to the nucleus of a target cell, the method comprising providing a modified AAV having a capsid comprising: (i) (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation; (ii) (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; or (iii) (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations; wherein the amino acid substitution mutations are numbered relative to the AAV9 major capsid protein VP1 (SEQ ID NO: 1).

In another aspect, the disclosure features a method of transduction of a target cell by an AAV at a lower dose of the AAV, the method comprising providing a modified AAV having a capsid comprising: (i) (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation; (ii) (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; or (iii) (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations; wherein the amino acid substitution mutations are numbered relative to the AAV9 major capsid protein VP1 (SEQ ID NO: 1).

In some embodiments, the method comprises contacting a target cell with the modified AAV.

In some aspects, the target cell is an endothelial cell, a cancer cell, a central nervous system (CNS) cell, an eye cell, a retinal cell, a muscle cell, a stem cell, a heart cell, a lung cell, a skin cell, a kidney cell, or a liver cell. In some aspects, the endothelial cell is a brain endothelial cell.

In some aspects, the major capsid protein VP1, minor capsid protein VP2, and/or minor capsid protein VP3 comprise one or more further amino acid substitution mutations and/or comprise one or more amino acid insertions or deletions.

In some aspects, the serotype of the modified AAV is AAV1, AAV2, AAV3, AAV6, AAV7, AAV8, AAV9, AAV10, AAV11, or AAV12. In some aspects, the serotype of the modified AAV is AAV9 or a variant thereof.

In another aspect, the disclosure features use of a modified AAV in the manufacture of a medicament for transducing a target cell, wherein the modified AAV has a capsid comprising: (i) (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation; (ii) (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; or (iii) (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations; wherein the amino acid substitution mutations are numbered relative to the AAV9 major capsid protein VP1 (SEQ ID NO: 1); and wherein the transduction affinity of the modified AAV is improved relative to the transduction efficiency of an AAV having a capsid not comprising any of the indicated amino acid substitution mutations.

In another aspect, the disclosure features use of a modified AAV in the manufacture of a medicament for delivering a cargo to a target cell, wherein the modified AAV has a capsid comprising: (i) (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation; (ii) (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; or (iii) (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations; wherein the cargo is encapsulated by the AAV; wherein the amino acid substitution mutations are numbered relative to the AAV9 major capsid protein VP1 (SEQ ID NO: 1); and wherein the transduction affinity of the modified AAV is improved relative to the transduction efficiency of an AAV having a capsid not comprising any of the indicated amino acid substitution mutations.

In another aspect, the disclosure features a modified AAV for use in transducing a target cell, wherein the modified AAV has a capsid comprising: (i) (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation; (ii) (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; or (iii) (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations; wherein the amino acid substitution mutations are numbered relative to the AAV9 major capsid protein VP1 (SEQ ID NO: 1); and wherein the transduction affinity of the modified AAV is improved relative to the transduction efficiency of an AAV having a capsid not comprising any of the indicated amino acid substitution mutations.

In another aspect, the disclosure features a modified AAV for use in delivering a cargo to a target cell, wherein the modified AAV has a capsid comprising (i) (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation; (ii) (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; or (iii) (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations; wherein the cargo is encapsulated by the AAV; wherein the amino acid substitution mutations are numbered relative to the AAV9 major capsid protein VP1 (SEQ ID NO: 1); and wherein the transduction affinity of the modified AAV is improved relative to the transduction efficiency of an AAV having a capsid not comprising any of the indicated amino acid substitution mutations.

In another aspect, the disclosure features an AAV having a capsid comprising (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation; wherein the amino acid substitution mutations are numbered relative to the AAV9 major capsid protein VP1 (SEQ ID NO: 1).

In some aspects, (a) the major capsid protein VP1 further comprises an A273N amino acid substitution mutation; (b) the minor capsid protein VP2 further comprises an A273N amino acid substitution mutation; and/or (c) the minor capsid protein VP3 further comprises an A273N amino acid substitution mutation.

In some aspects, (a) the major capsid protein VP1 further comprises a Q387K amino acid substitution mutation; (b) the minor capsid protein VP2 further comprises a Q387K amino acid substitution mutation; and/or (c) the minor capsid protein VP3 further comprises a Q387K amino acid substitution mutation.

In some aspects, (a) the major capsid protein VP1 further comprises an E500R amino acid substitution mutation; (b) the minor capsid protein VP2 further comprises an E500R amino acid substitution mutation; and/or (c) the minor capsid protein VP3 further comprises an E500R amino acid substitution mutation.

In some aspects, (a) the major capsid protein VP1 further comprises an E500P amino acid substitution mutation; (b) the minor capsid protein VP2 further comprises an E500P amino acid substitution mutation; and/or (c) the minor capsid protein VP3 further comprises an E500P amino acid substitution mutation.

In some aspects, (a) the major capsid protein VP1 further comprises A273N, Q387K, and E500R amino acid substitution mutations; (b) the minor capsid protein VP2 further comprises A273N, Q387K, and E500R amino acid substitution mutations; and/or (c) the minor capsid protein VP3 further comprises A273N, Q387K, and E500R amino acid substitution mutations.

In another aspect, the disclosure features an AAV having a capsid comprising (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; wherein the amino acid substitution mutations are numbered relative to the AAV9 major capsid protein VP1 (SEQ ID NO: 1).

In another aspect, the disclosure features an AAV having a capsid comprising: (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations; wherein the amino acid substitution mutations are numbered relative to the AAV9 major capsid protein VP1 (SEQ ID NO: 1).

In some aspects, the serotype of the AAV is AAV1, AAV2, AAV3, AAV6, AAV7, AAV8, AAV9, AAV10, AAV11, or AAV12. In some aspects, the serotype of the modified AAV is AAV9 or a variant thereof.

In another aspect, the disclosure features a method of improving transduction efficiency of an adeno-associated virus (AAV), the method comprising providing a modified AAV having a capsid comprising (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; wherein the AAV is AAV9 or a variant thereof and the amino acid substitution mutations are numbered relative to the AAV9 major capsid protein VP1 (SEQ ID NO: 1).

In another aspect, the disclosure features a method of improving transduction efficiency of an adeno-associated virus (AAV), the method comprising contacting a target cell with a modified AAV having a capsid comprising (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; wherein the AAV is AAV9 or a variant thereof and the amino acid substitution mutations are numbered relative to the AAV9 major capsid protein VP1 (SEQ ID NO: 1), thereby improving transduction efficiency.

In some aspects, (a) the major capsid protein VP1 further comprises an A273N amino acid substitution mutation; (b) the minor capsid protein VP2 further comprises an A273N amino acid substitution mutation; and/or (c) the minor capsid protein VP3 further comprises an A273N amino acid substitution mutation.

In some aspects, (a) the major capsid protein VP1 further comprises a Q387K amino acid substitution mutation; (b) the minor capsid protein VP2 further comprises a Q387K amino acid substitution mutation; and/or (c) the minor capsid protein VP3 further comprises a Q387K amino acid substitution mutation.

In some aspects, (a) the major capsid protein VP1 further comprises an A472H amino acid substitution mutation; (b) the minor capsid protein VP2 further comprises an A472H amino acid substitution mutation; and/or (c) the minor capsid protein VP3 further comprises an A472H amino acid substitution mutation.

In some aspects, (a) the major capsid protein VP1 further comprises A273N, Q387K, and A472H amino acid substitution mutations; (b) the minor capsid protein VP2 further comprises A273N, Q387K, and A472H amino acid substitution mutations; and/or (c) the minor capsid protein VP3 further comprises A273N, Q387K, and A472H amino acid substitution mutations.

In another aspect, the disclosure features a method of improving transduction efficiency of an AAV, the method comprising providing a modified AAV having a capsid comprising (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations; wherein the AAV is AAV9 or a variant thereof and the amino acid substitution mutations are numbered relative to the AAV9 major capsid protein VP1 (SEQ ID NO: 1).

In another aspect, the disclosure features a method of improving transduction efficiency of an AAV, the method comprising contacting a target cell with a modified AAV having a capsid comprising (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations; wherein the AAV is AAV9 or a variant thereof and the amino acid substitution mutations are numbered relative to the AAV9 major capsid protein VP1 (SEQ ID NO: 1), thereby improving transduction efficiency.

In some aspects, (a) the major capsid protein VP1 further comprises an A472H amino acid substitution mutation; (b) the minor capsid protein VP2 further comprises an A472H amino acid substitution mutation; and/or (c) the minor capsid protein VP3 further comprises an A472H amino acid substitution mutation.

In some aspects, the method comprises contacting a target cell with the modified AAV, thereby improving transduction efficiency.

In some aspects, the transduction efficiency is improved relative to the transduction efficiency of an AAV9 having a capsid not comprising any of the indicated amino acid substitution mutations.

In another aspect, the disclosure features a method of delivering a cargo to a cell, the method comprising contacting a target cell with a modified AAV having a capsid comprising (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; wherein the cargo is encapsulated by the AAV; wherein the AAV is AAV9 or a variant thereof and the amino acid substitution mutations are numbered relative to the AAV9 major capsid protein VP1 (SEQ ID NO: 1); and wherein the AAV has an increased rate of transduction into a target cell relative to an AAV9 having a capsid not comprising any of the indicated amino acid substitution mutations.

In some aspects, (a) the major capsid protein VP1 further comprises an A273N amino acid substitution mutation; (b) the minor capsid protein VP2 further comprises an A273N amino acid substitution mutation; and/or (c) the minor capsid protein VP3 further comprises an A273N amino acid substitution mutation.

In some aspects, (a) the major capsid protein VP1 further comprises a Q387K amino acid substitution mutation; (b) the minor capsid protein VP2 further comprises a Q387K amino acid substitution mutation; and/or (c) the minor capsid protein VP3 further comprises a Q387K amino acid substitution mutation.

In some aspects, (a) the major capsid protein VP1 further comprises an A472H amino acid substitution mutation; (b) the minor capsid protein VP2 further comprises an A472H amino acid substitution mutation; and/or (c) the minor capsid protein VP3 further comprises an A472H amino acid substitution mutation.

In some aspects, (a) the major capsid protein VP1 further comprises A273N, Q387K, and A472H amino acid substitution mutations; (b) the minor capsid protein VP2 further comprises A273N, Q387K, and A472H amino acid substitution mutations; and/or (c) the minor capsid protein VP3 further comprises A273N, Q387K, and A472H amino acid substitution mutations.

In another aspect, the disclosure features a method of delivering a cargo to a cell, the method comprising contacting a target cell with a modified AAV having a capsid comprising (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations; wherein the cargo is encapsulated by the AAV; wherein the AAV is AAV9 or a variant thereof and the amino acid substitution mutations are numbered relative to the AAV9 major capsid protein VP1 (SEQ ID NO: 1); and wherein the AAV has an increased rate of transduction into a target cell relative to an AAV9 having a capsid not comprising any of the indicated amino acid substitution mutations.

In some aspects, (a) the major capsid protein VP1 further comprises an A472H amino acid substitution mutation; (b) the minor capsid protein VP2 further comprises an A472H amino acid substitution mutation; and/or (c) the minor capsid protein VP3 further comprises an A472H amino acid substitution mutation.

In some aspects, the modified AAV has an increased affinity for the AAV receptor (AAVR) relative to an AAV9 having a capsid that does not comprise any of the indicated amino acid substitution mutations.

In some aspects, the modified AAV has an increased rate of delivery to the nucleus of the target cell relative to an AAV9 having a capsid that does not comprise any of the indicated amino acid substitution mutations.

In some aspects, the modified AAV transduces the target cell at a lower dose than an AAV9 having a capsid that does not comprise any of the indicated amino acid substitution mutations.

In another aspect, the disclosure features a method of improving the affinity of an AAV for AAV receptor (AAVR), the method comprising providing a modified AAV having a capsid comprising: (i) (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; or (ii) (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations; wherein the AAV is AAV9 or a variant thereof and the amino acid substitution mutations are numbered relative to the AAV9 major capsid protein VP1 (SEQ ID NO: 1).

In another aspect, the disclosure features a method of increasing the rate of delivery of an AAV to the nucleus of a target cell, the method comprising providing a modified AAV having a capsid comprising: (i) (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; or (ii) (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations; wherein the AAV is AAV9 or a variant thereof and the amino acid substitution mutations are numbered relative to the AAV9 major capsid protein VP1 (SEQ ID NO: 1).

In another aspect, the disclosure features a method of transduction of a target cell by an AAV at a lower dose of the AAV, the method comprising providing a modified AAV having a capsid comprising: (i) (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; or (ii) (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations; wherein the AAV is AAV9 or a variant thereof and the amino acid substitution mutations are numbered relative to the AAV9 major capsid protein VP1 (SEQ ID NO: 1).

In some aspects, the method comprises contacting a target cell with the modified AAV.

In some aspects, the target cell is an endothelial cell, a cancer cell, a central nervous system (CNS) cell, an eye cell, a retinal cell, a muscle cell, a stem cell, a heart cell, a lung cell, a skin cell, a kidney cell, or a liver cell. In some aspects, the endothelial cell is a brain endothelial cell.

In some aspects, the major capsid protein VP1, minor capsid protein VP2, and/or minor capsid protein VP3 comprise one or more further amino acid substitution mutations and/or comprise one or more amino acid insertions or deletions.

In another aspect, the disclosure features use of a modified AAV in the manufacture of a medicament for transducing a target cell, wherein the modified AAV has a capsid comprising: (i) (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; or (ii) (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations; wherein the AAV is AAV9 or a variant thereof and the amino acid substitution mutations are numbered relative to the AAV9 major capsid protein VP1 (SEQ ID NO: 1); and wherein the transduction affinity of the modified AAV is improved relative to the transduction efficiency of an AAV having a capsid not comprising any of the indicated amino acid substitution mutations.

In another aspect, the disclosure features use of a modified AAV in the manufacture of a medicament for delivering a cargo to a target cell, wherein the modified AAV has a capsid comprising: (i) (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; or (ii) (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations; wherein the cargo is encapsulated by the AAV; wherein the AAV is AAV9 or a variant thereof and the amino acid substitution mutations are numbered relative to the AAV9 major capsid protein VP1 (SEQ ID NO: 1); and wherein the transduction affinity of the modified AAV is improved relative to the transduction efficiency of an AAV having a capsid not comprising any of the indicated amino acid substitution mutations.

In another aspect, the disclosure features a modified AAV for use in transducing a target cell, wherein the modified AAV has a capsid comprising: (i) (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; or (ii) (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations; wherein the AAV is AAV9 or a variant thereof and the amino acid substitution mutations are numbered relative to the AAV9 major capsid protein VP1 (SEQ ID NO: 1); and wherein the transduction affinity of the modified AAV is improved relative to the transduction efficiency of an AAV having a capsid not comprising any of the indicated amino acid substitution mutations.

In another aspect, the disclosure features a modified AAV for use in delivering a cargo to a target cell, wherein the modified AAV has a capsid comprising: (i) (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; or (ii) (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations; wherein the cargo is encapsulated by the AAV; wherein the AAV is AAV9 or a variant thereof and the amino acid substitution mutations are numbered relative to the AAV9 major capsid protein VP1 (SEQ ID NO: 1); and wherein the transduction affinity of the modified AAV is improved relative to the transduction efficiency of an AAV having a capsid not comprising any of the indicated amino acid substitution mutations.

In another aspect, the disclosure features an AAV having a capsid comprising (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; wherein the AAV is AAV9 or a variant thereof and the amino acid substitution mutations are numbered relative to the AAV9 major capsid protein VP1 (SEQ ID NO: 1).

In some aspects, (a) the major capsid protein VP1 further comprises an A273N amino acid substitution mutation; (b) the minor capsid protein VP2 further comprises an A273N amino acid substitution mutation; and/or (c) the minor capsid protein VP3 further comprises an A273N amino acid substitution mutation.

In some aspects, (a) the major capsid protein VP1 further comprises a Q387K amino acid substitution mutation; (b) the minor capsid protein VP2 further comprises a Q387K amino acid substitution mutation; and/or (c) the minor capsid protein VP3 further comprises a Q387K amino acid substitution mutation.

In some aspects, (a) the major capsid protein VP1 further comprises an A472H amino acid substitution mutation; (b) the minor capsid protein VP2 further comprises an A472H amino acid substitution mutation; and/or (c) the minor capsid protein VP3 further comprises an A472H amino acid substitution mutation.

In some aspects, (a) the major capsid protein VP1 further comprises A273N, Q387K, and A472H amino acid substitution mutations; (b) the minor capsid protein VP2 further comprises A273N, Q387K, and A472H amino acid substitution mutations; and/or (c) the minor capsid protein VP3 further comprises A273N, Q387K, and A472H amino acid substitution mutations.

In another aspect, the disclosure features an AAV having a capsid comprising (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations; wherein the AAV is AAV9 or a variant thereof and the amino acid substitution mutations are numbered relative to the AAV9 major capsid protein VP1 (SEQ ID NO: 1).

In another aspect, (a) the major capsid protein VP1 further comprises an A472H amino acid substitution mutation; (b) the minor capsid protein VP2 further comprises an A472H amino acid substitution mutation; and/or (c) the minor capsid protein VP3 further comprises an A472H amino acid substitution mutation.

In another aspect, the disclosure features a method of improving transduction efficiency of an adeno-associated virus (AAV), the method comprising providing a modified AAV having a capsid comprising (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations; wherein the AAV is AAV2 or a variant thereof and the amino acid substitution mutations are numbered relative to the AAV2 major capsid protein VP1 (SEQ ID NO: 2).

In some aspects, the method comprises contacting a target cell with the modified AAV, thereby improving transduction efficiency.

In another aspect, the disclosure features a method of improving transduction efficiency of an adeno-associated virus (AAV), the method comprising contacting a target cell with a modified AAV having a capsid comprising (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations; wherein the AAV is AAV2 or a variant thereof and the amino acid substitution mutations are numbered relative to the AAV2 major capsid protein VP1 (SEQ ID NO: 2), thereby improving transduction efficiency.

In some aspects, the transduction efficiency is improved relative to the transduction efficiency of an AAV2 having a capsid not comprising either of the indicated amino acid substitution mutations.

In another aspect, the disclosure features a method of delivering a cargo to a cell, the method comprising contacting a target cell with a modified AAV having a capsid comprising (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations; wherein the cargo is encapsulated by the AAV; wherein the AAV is AAV2 or a variant thereof and the amino acid substitution mutations are numbered relative to the AAV2 major capsid protein VP1 (SEQ ID NO: 2); and wherein the AAV has an increased rate of transduction into a target cell relative to an AAV2 having a capsid not comprising either of the indicated amino acid substitution mutations.

In some aspects, the modified AAV has an increased affinity for the AAV receptor (AAVR) relative to an AAV2 having a capsid that does not comprise either of the indicated amino acid substitution mutations.

In some aspects, the modified AAV has an increased rate of delivery to the nucleus of the target cell relative to an AAV2 having a capsid that does not comprise either of the indicated amino acid substitution mutations.

In some aspects, the modified AAV transduces the target cell at a lower dose than an AAV2 having a capsid that does not comprise either of the indicated amino acid substitution mutations.

In another aspect, the disclosure features a method of improving the affinity of an AAV for the AAV receptor (AAVR), the method comprising providing a modified AAV having a capsid comprising: (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations; wherein the AAV is AAV2 or a variant thereof and the amino acid substitution mutations are numbered relative to the AAV2 major capsid protein VP1 (SEQ ID NO: 2).

In another aspect, the disclosure features a method of increasing the rate of delivery of an AAV to the nucleus of a target cell, the method comprising providing a modified AAV having a capsid comprising: (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations; wherein the AAV is AAV2 or a variant thereof and the amino acid substitution mutations are numbered relative to the AAV2 major capsid protein VP1 (SEQ ID NO: 2).

In another aspect, the disclosure features a method of transducing a target cell by an AAV at a lower dose of the AAV, the method comprising providing a modified AAV having a capsid comprising: (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations; wherein the AAV is AAV2 or a variant thereof and the amino acid substitution mutations are numbered relative to the AAV2 major capsid protein VP1 (SEQ ID NO: 2).

In some aspects, the method comprises contacting a target cell with the modified AAV.

In some aspects, the target cell is an endothelial cell, a cancer cell, a CNS cell, an eye cell, a retinal cell, a muscle cell, a stem cell, a heart cell, a lung cell, a skin cell, a kidney cell, or a liver cell. In some aspects, the endothelial cell is a brain endothelial cell.

In some aspects, the major capsid protein VP1, minor capsid protein VP2, and/or minor capsid protein VP3 comprise one or more further amino acid substitution mutations and/or comprise one or more amino acid insertions or deletions.

In another aspect, the disclosure features use of a modified AAV in the manufacture of a medicament for transducing a target cell, wherein the modified AAV has a capsid comprising: (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations; wherein the AAV is AAV2 or a variant thereof and the amino acid substitution mutations are numbered relative to the AAV2 major capsid protein VP1 (SEQ ID NO: 2), and wherein the transduction affinity of the modified AAV is improved relative to the transduction efficiency of an AAV having a capsid not comprising any of the indicated amino acid substitution mutations.

In another aspect, the disclosure features use of a modified AAV in the manufacture of a medicament for delivering a cargo to a target cell, wherein the modified AAV has a capsid comprising: (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations; wherein the cargo is encapsulated by the AAV, wherein the AAV is AAV2 or a variant thereof and the amino acid substitution mutations are numbered relative to the AAV2 major capsid protein VP1 (SEQ ID NO: 2), and wherein the transduction affinity of the modified AAV is improved relative to the transduction efficiency of an AAV having a capsid not comprising any of the indicated amino acid substitution mutations.

In another aspect, the disclosure features a modified AAV for use in transducing a target cell, wherein the modified AAV has a capsid comprising: (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations; wherein the AAV is AAV2 or a variant thereof and the amino acid substitution mutations are numbered relative to the AAV2 major capsid protein VP1 (SEQ ID NO: 2), and wherein the transduction affinity of the modified AAV is improved relative to the transduction efficiency of an AAV having a capsid not comprising any of the indicated amino acid substitution mutations.

In another aspect, the disclosure features a modified AAV for use in delivering a cargo to a target cell, wherein the modified AAV has a capsid comprising: (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations; wherein the AAV is AAV2 or a variant thereof and the amino acid substitution mutations are numbered relative to the AAV2 major capsid protein VP1 (SEQ ID NO: 2), and wherein the transduction affinity of the modified AAV is improved relative to the transduction efficiency of an AAV having a capsid not comprising any of the indicated amino acid substitution mutations.

In another aspect, the disclosure features an AAV having a capsid comprising (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations; wherein the AAV is AAV2 or a variant thereof and the amino acid substitution mutations are numbered relative to the AAV2 major capsid protein VP1 (SEQ ID NO: 2).

In another aspect, the disclosure features a method of improving transduction efficiency of an adeno-associated virus (AAV), the method comprising providing a modified AAV having a capsid comprising: (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations; wherein the AAV is AAV9 or a variant thereof and the amino acid substitution mutations are numbered relative to the AAV9 major capsid protein VP1 (SEQ ID NO: 1).

In another aspect, the disclosure features a method of improving transduction efficiency of an adeno-associated virus (AAV), the method comprising contacting a target cell with a modified AAV having a capsid comprising: (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations; wherein the AAV is AAV9 or a variant thereof and the amino acid substitution mutations are numbered relative to the AAV9 major capsid protein VP1 (SEQ ID NO: 1), thereby improving transduction efficiency.

In some aspects, (a) the major capsid protein VP1 further comprises an E500P amino acid substitution mutation; (b) the minor capsid protein VP2 further comprises an E500P amino acid substitution mutation; and/or (c) the minor capsid protein VP3 further comprises an E500P amino acid substitution mutation.

In some aspects, (a) the major capsid protein VP1 further comprises an E500R amino acid substitution mutation; (b) the minor capsid protein VP2 further comprises an E500R amino acid substitution mutation; and/or (c) the minor capsid protein VP3 further comprises an E500R amino acid substitution mutation.

In another aspect, the disclosure features a method of improving transduction efficiency of an AAV, the method comprising contacting a target cell with a modified AAV having a capsid comprising: (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; wherein the AAV is AAV9 or a variant thereof and the amino acid substitution mutations are numbered relative to the AAV9 major capsid protein VP1 (SEQ ID NO: 1), thereby increasing transduction efficiency.

In another aspect, the disclosure features a method of improving transduction efficiency of an AAV, the method comprising providing a modified AAV having a capsid comprising: (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; wherein the AAV is AAV9 or a variant thereof and the amino acid substitution mutations are numbered relative to the AAV9 major capsid protein VP1 (SEQ ID NO: 1).

In another aspect, the disclosure features a method of improving transduction efficiency of an AAV, the method comprising contacting a target cell with a modified AAV having a capsid comprising: (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations; wherein the AAV is AAV9 or a variant thereof and the amino acid substitution mutations are numbered relative to the AAV9 major capsid protein VP1 (SEQ ID NO: 1), thereby increasing transduction efficiency.

In another aspect, the disclosure features a method of improving transduction efficiency of an AAV, the method comprising providing a modified AAV having a capsid comprising: (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations; wherein the AAV is AAV9 or a variant thereof and the amino acid substitution mutations are numbered relative to the AAV9 major capsid protein VP1 (SEQ ID NO: 1).

In some embodiments, the method comprises contacting a target cell with the modified AAV, thereby improving transduction efficiency.

In some embodiments, the transduction efficiency is improved relative to the transduction efficiency of an AAV9 having a capsid not comprising any of the indicated amino acid substitution mutations.

In another aspect, the disclosure features a method of delivering a cargo to a cell, the method comprising contacting a target cell with a modified AAV having a capsid comprising: (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations; wherein the cargo is encapsulated by the AAV; wherein the AAV is AAV9 or a variant thereof and the amino acid substitution mutations are numbered relative to the AAV9 major capsid protein VP1 (SEQ ID NO: 1); and wherein the AAV has an increased rate of transduction into a target cell relative to an AAV9 having a capsid not comprising any of the indicated amino acid substitution mutations.

In some aspects, (a) the major capsid protein VP1 further comprises an E500P amino acid substitution mutation; (b) the minor capsid protein VP2 further comprises an E500P amino acid substitution mutation; and/or (c) the minor capsid protein VP3 further comprises an E500P amino acid substitution mutation.

In some aspects, (a) the major capsid protein VP1 further comprises an E500R amino acid substitution mutation; (b) the minor capsid protein VP2 further comprises an E500R amino acid substitution mutation; and/or (c) the minor capsid protein VP3 further comprises an E500R amino acid substitution mutation.

In another aspect, the disclosure features a method of delivering a cargo to a cell, the method comprising contacting a target cell with a modified AAV having a capsid comprising: (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; wherein the cargo is encapsulated by the AAV; wherein the AAV is AAV9 or a variant thereof and the amino acid substitution mutations are numbered relative to the AAV9 major capsid protein VP1 (SEQ ID NO: 1); and wherein the AAV has an increased rate of transduction into a target cell relative to an AAV9 having a capsid not comprising any of the indicated amino acid substitution mutations.

In another aspect, the disclosure features a method of delivering a cargo to a cell, the method comprising contacting a target cell with a modified AAV having a capsid comprising: (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations; wherein the cargo is encapsulated by the AAV; wherein the AAV is AAV9 or a variant thereof and the amino acid substitution mutations are numbered relative to the AAV9 major capsid protein VP1 (SEQ ID NO: 1); and wherein the AAV has an increased rate of transduction into a target cell relative to an AAV9 having a capsid not comprising any of the indicated amino acid substitution mutations.

In some aspects, the modified AAV has an increased affinity for AAV receptor (AAVR) relative to an AAV9 having a capsid that does not comprise any of the indicated amino acid substitution mutations.

In some aspects, the modified AAV has an increased rate of delivery to the nucleus of the target cell relative to an AAV9 having a capsid that does not comprise any of the indicated amino acid substitution mutations.

In some aspects, the modified AAV transduces the target cell at a lower dose than an AAV9 having a capsid that does not comprise any of the indicated amino acid substitution mutations.

In another aspect, the disclosure features a method of improving the affinity of an AAV for AAV receptor (AAVR), the method comprising providing a modified AAV having a capsid comprising: (i) (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations; (ii) (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; or (iii) (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations; wherein the AAV is AAV9 or a variant thereof and the amino acid substitution mutations are numbered relative to the AAV9 major capsid protein VP1 (SEQ ID NO: 1).

In another aspect, the disclosure features a method of increasing the rate of delivery of an AAV to the nucleus of a target cell, the method comprising providing a modified AAV having a capsid comprising: (i) (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations; (ii) (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; or (iii) (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations; wherein the AAV is AAV9 or a variant thereof and the amino acid substitution mutations are numbered relative to the AAV9 major capsid protein VP1 (SEQ ID NO: 1).

In another aspect, the disclosure features a method of transduction of a target cell by an AAV at a lower dose of the AAV, the method comprising providing a modified AAV having a capsid comprising: (i) (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations; (ii) (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; or (iii) (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations; wherein the AAV is AAV9 or a variant thereof and the amino acid substitution mutations are numbered relative to the AAV9 major capsid protein VP1 (SEQ ID NO: 1).

In some aspects, the method comprises contacting a target cell with the modified AAV.

In some aspects, the target cell is an endothelial cell, a cancer cell, a central nervous system (CNS) cell, an eye cell, a retinal cell, a muscle cell, a stem cell, a heart cell, a lung cell, a skin cell, a kidney cell, or a liver cell. In some aspects, the endothelial cell is a brain endothelial cell.

In some aspects, the major capsid protein VP1, minor capsid protein VP2, and/or minor capsid protein VP3 comprise one or more further amino acid substitution mutations and/or comprise one or more amino acid insertions or deletions.

In another aspect, the disclosure features use of a modified AAV in the manufacture of a medicament for transducing a target cell, wherein the modified AAV has a capsid comprising: (i) (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations; (ii) (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; or (iii) (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations; wherein the AAV is AAV9 or a variant thereof and the amino acid substitution mutations are numbered relative to the AAV9 major capsid protein VP1 (SEQ ID NO: 1); and wherein the transduction affinity of the modified AAV is improved relative to the transduction efficiency of an AAV having a capsid not comprising any of the indicated amino acid substitution mutations.

In another aspect, the disclosure features use of a modified AAV in the manufacture of a medicament for delivering a cargo to a target cell, wherein the modified AAV has a capsid comprising: (i) (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations; (ii) (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; or (iii) (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations; wherein the cargo is encapsulated by the AAV; wherein the AAV is AAV9 or a variant thereof and the amino acid substitution mutations are numbered relative to the AAV9 major capsid protein VP1 (SEQ ID NO: 1); and wherein the transduction affinity of the modified AAV is improved relative to the transduction efficiency of an AAV having a capsid not comprising any of the indicated amino acid substitution mutations.

In another aspect, the disclosure features a modified AAV for use in transducing a target cell, wherein the modified AAV has a capsid comprising (i) (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations; (ii) (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; or (iii) (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations; wherein the AAV is AAV9 or a variant thereof and the amino acid substitution mutations are numbered relative to the AAV9 major capsid protein VP1 (SEQ ID NO: 1); and wherein the transduction affinity of the modified AAV is improved relative to the transduction efficiency of an AAV having a capsid not comprising any of the indicated amino acid substitution mutations.

In another aspect, the disclosure features a modified AAV for use in delivering a cargo to a target cell, wherein the modified AAV has a capsid comprising: (i) (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations; (ii) (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; or (iii) (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations; wherein the cargo is encapsulated by the AAV; wherein the AAV is AAV9 or a variant thereof and the amino acid substitution mutations are numbered relative to the AAV9 major capsid protein VP1 (SEQ ID NO: 1); and wherein the transduction affinity of the modified AAV is improved relative to the transduction efficiency of an AAV having a capsid not comprising any of the indicated amino acid substitution mutations.

In another aspect, the disclosure features an AAV having a capsid comprising: (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations; wherein the AAV is AAV9 or a variant thereof and the amino acid substitution mutations are numbered relative to the AAV9 major capsid protein VP1 (SEQ ID NO: 1).

In some aspects, (a) the major capsid protein VP1 further comprises an E500P amino acid substitution mutation; (b) the minor capsid protein VP2 further comprises an E500P amino acid substitution mutation; and/or (c) the minor capsid protein VP3 further comprises an E500P amino acid substitution mutation.

In some aspects, (a) the major capsid protein VP1 further comprises an E500R amino acid substitution mutation; (b) the minor capsid protein VP2 further comprises an E500R amino acid substitution mutation; and/or (c) the minor capsid protein VP3 further comprises an E500R amino acid substitution mutation.

In another aspect, the disclosure features an AAV having a capsid comprising: (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; wherein the AAV is AAV9 or a variant thereof and the amino acid substitution mutations are numbered relative to the AAV9 major capsid protein VP1 (SEQ ID NO: 1).

In another aspect, the disclosure features an AAV having a capsid comprising: (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations; wherein the AAV is AAV9 or a variant thereof and the amino acid substitution mutations are numbered relative to the AAV9 major capsid protein VP1 (SEQ ID NO: 1). In another aspect, the disclosure features a method for determining the affinity of an AAV capsid for a query protein, the method comprising (a) providing a virus-like particle (VLP) having a capsid consisting essentially of the VP3 subunit of the AAV capsid; (b) contacting the VLP of step (a) with the query protein under conditions permitting the binding of the query protein and the VLP; and (c) quantifying the strength of the interaction between the query protein and the VLP, thereby determining the affinity of the AAV capsid for the query protein.

In some aspects, the VLP is immobilized on a solid surface. In some aspects, the solid surface comprises an antibody or antigen-binding fragment thereof having affinity for the VLP. In some aspects, the antibody is a single-domain antibody. In some aspects, the antigen-binding fragment is a Fab. In some aspects, the antibody or antigen-binding fragment thereof binds to the VP3 subunit of the AAV capsid.

In some aspects, the VLP is produced in a cell line, and immobilizing the VLP on the solid surface comprises contacting the solid surface with a culture medium comprising the VLP. In some aspects, the culture medium comprises lysate of the cell line from which the VLP is produced.

In some aspects, the quantifying is performed using biolayer interferometry (BLI) and/or surface plasmon resonance (SPR).

In some aspects, the VP3 subunit of the AAV capsid comprises one or more amino acid substitution mutations relative to the sequence of a wild-type VP3 subunit of the AAV capsid.

In some aspects, the serotype of the AAV is AAV1, AAV2, AAV3, AAV4, AAV5, AAV6, AAV7, AAV8, AAV9, AAV10, AAV11, or AAV12.

### BRIEF DESCRIPTION OF THE DRAWINGS

FIG. 1 is a schematic diagram showing a workflow for screening AAV virus-like particle (VLP) mutants comprising variants of the minor capsid protein VP3 for affinity to a target molecule. BLI: biolayer interferometry. SPR: surface plasmon resonance.

FIG. 2A is a chart showing amino acid substitution mutations made at residues at the AAV2 or AAV9 capsid/AAVR interface.

FIG. 2B is a pair of protein structure diagrams showing interactions between residue 385 of the AAV2 capsid and residue 437 of the AAV receptor (AAVR; SEQ ID NO: 3). The left panel shows an AAV2 capsid protein having the native residue Q at position 385 (Q385), which forms a hydrogen bond (H-bond) with D437 of AAVR. The right panel shows an AAV2 capsid protein having a K at position 385 (Q385K substitution mutation), which may form a salt bridge with D437 of AAVR.

FIG. 2C is a pair of protein structure diagrams showing interactions between residue 271 of the AAV2 capsid and residue 437 of AAVR. The left panel shows an AAV2 capsid protein having the native residue H at position 271 (H271). The right panel shows an AAV2 capsid protein having a N at position 271 (H271N substitution mutation), which may form a new backbone H-bond with D437 of AAVR.

FIG. 3A is a schematic diagram showing a workflow for assessing affinity of VLPs for AAVR using BLI.

FIG. 3B is a sensorgram showing the results of a BLI experiment in which AAV2 VLPs comprising variants of the minor capsid protein VP3 were assessed for affinity to AAVR.

FIG. 3C is a set of sensorgrams showing affinity of wild-type (WT) AAV2 VLPs and AAV2 VLPs comprising T592D, A593E, and T592D/A593E amino acid substitution mutations (numbered relative to AAV2 VP1) for AAVR, as assessed using SPR.

FIG. 4A is a sensorgram showing the results of a BLI experiment in which AAV9 VLPs comprising variants of the minor capsid protein VP3 were assessed for affinity to AAVR.

FIG. 4B is a set of sensorgrams showing affinity of WT AAV9 VLPs and AAV9 VLPs comprising A273N, Q387K, and E500R amino acid substitution mutations (numbered relative to AAV9 VP1) for AAVR, as assessed using SPR.

FIG. 5 is a schematic diagram showing the location of the A273 residue in the major capsid protein VP1 and the minor capsid proteins VP2 and VP3; the location of the related P98 residue in the accessory protein assembly-activating protein (AAP); the effect of introducing an A273N mutation (via replacement of the codon GCC with either AAT or AAC) on the P98 codon; and an alignment of AAP protein sequences from the indicated AAV serotypes.

FIG. 6 is a chart showing capture levels from conditioned media of AAV9 VLPs generated with co-expressed AAP mutants on anti-AAV9 BLI biosensor.

FIG. 7A is a bar graph showing the yield titer (vector genome (vg)/mL) of wild-type AAV9 (AAV9) and AAV9 comprising a Q387K amino acid substitution mutation (AAV9-Q387K) in 40 mL EXPI293™ cell culture.

FIG. 7B is a pair of bar graphs showing the transduction efficiency of wild-type AAV9 and AAV9 comprising a Q387K amino acid substitution mutation in MDA-MB-231 cells (percent of cells). Left panel: AAVs derived from cell pellet; right panel: AAVs derived from culture medium. MOI: multiplicity of infection.

FIG. 7C is a bar graph showing the transduction efficiency of wild-type AAV9 and AAV9 comprising a Q387K amino acid substitution mutation in GNE293T (293/tsA1609neo) cells (percent of cells).

FIG. 7D is a bar graph showing the transduction efficiency of wild-type AAV9 and AAV9 comprising a Q387K amino acid substitution mutation in wild-type HEK293 cells; in a cell line not comprising AAVR or GPR108 (DKO-25); and in a cell line not comprising AAVR (KIAA0319L-1).

FIG. 8 is a set of sensorgrams showing affinity of WT AAV9 VLPs and AAV9 VLPs comprising Q387K; A273N and Q387K; A273N and E500R; Q387K and E500R; and A273N, Q387K, and E500R amino acid substitution mutations for AAVR, as assessed using SPR.

FIG. 9 is a set of sensorgrams showing affinity of WT AAV9 VLPs and AAV9 VLPs comprising A273N, Q387K, E500R, and A472H amino acid substitution mutations (numbered relative to AAV9 VP1) for AAVR, as assessed using SPR.

FIG. 10A is a bar graph showing the transduction efficiency of a control AAV9 and AAV9 comprising an E500R (“ER”), A273N (“AN”), and/or Q387K (“QK”) amino acid substitution mutation and a GFP marker in HEK293T cells, as measured by the proportion of cells comprising the GFP marker. Multiplicity of infection (MOI): 1×10¹³.

FIG. 10B is a bar graph showing the transduction efficiency of a control AAV9 and AAV9 comprising an E500R (“ER”), A273N (“AN”), and/or Q387K (“QK”) amino acid substitution mutation and a GFP marker in HEK293T cells, as measured by the proportion of cells comprising the GFP marker. MOI: 1×10⁵.

FIG. 10C is a bar graph showing the transduction efficiency of a control AAV9 and AAV9 comprising an E500R (“ER”), A273N (“AN”), and/or Q387K (“QK”) amino acid substitution mutation and a GFP marker in HEK293 cells, as measured by geometric mean fluorescence intensity (Geo MFI) of a FITC marker. MOI: 1×10⁶.

FIG. 10D is a bar graph showing the transduction efficiency of a control AAV9 and AAV9 comprising an E500R (“ER”), A273N (“AN”), and/or Q387K (“QK”) amino acid substitution mutation and a GFP marker in HEK293 cells, as measured by Geo MFI of a FITC marker. MOI: 1×10⁵.

FIG. 10E is a set of photomicrographs showing the transduction efficiency of a control AAV9 and AAV9 comprising an E500R (“ER”), A273N (“AN”), and/or Q387K (“QK”) amino acid substitution mutation in HEK293T cells, as indicated by the proportion of cells comprising a GFP marker. MOI: 1×10⁵.

FIG. 11A is a bar graph showing the transduction efficiency of a control AAV9 and AAV9 comprising an E500R (“ER”), A273N (“AN”), and/or Q387K (“QK”) amino acid substitution mutation and a GFP marker in SH-SY5y (neuroblastoma) cells, as measured by the proportion of cells comprising the GFP marker. MOI: 1×10⁶.

FIG. 11B is a bar graph showing the transduction efficiency of a control AAV9 and AAV9 comprising an E500R (“ER”), A273N (“AN”), and/or Q387K (“QK”) amino acid substitution mutation and a GFP marker in SH-SY5y cells, as measured by the proportion of cells comprising the GFP marker. MOI: 1×10⁵.

FIG. 11C is a bar graph showing the transduction efficiency of a control AAV9 and AAV9 comprising an E500R (“ER”), A273N (“AN”), and/or Q387K (“QK”) amino acid substitution mutation and a GFP marker in SH-SY5y cells, as measured by Geo MFI of a FITC marker. Multipiicity of infection (MOI): 1×10⁶.

FIG. 11D is a bar graph showing the transduction efficiency of a control AAV9 and AAV9 comprising an E500R (“ER”), A273N (“AN”), and/or Q387K (“QK”) amino acid substitution mutation and a GFP marker in SH-SY5y cells, as measured by Geo MFI of a FITC marker. Multiplicity of infection (MOI): 1×10⁶.

FIG. 12 is a diagram showing the affinity for AAVR of VLPs comprising the indicated amino acid substitution mutations (presented as a ratio of the dissociation constant (K_{D}) of a wild-type VLP to the K_{D} of the mutant (K_{D} fold change)), as measured using the CARTERRA® LSA. An X denotes a mutant that was not tested. A blank space denotes a mutant for which a K_{D} fold change of less than 0.05 was observed. Fold changes between 0.05 and 0.1 are rounded to 0.1.

FIG. 13 is a pair of ribbon diagrams showing AAV9 in complex with AAVR (Protein Data Bank (PDB) 7WJX). The locations of six AAVR residues that were associated with improved affinity when mutated are shown.

FIG. 14 is a set of sensorgrams showing affinity of unpurified WT VLPs (from cell media) and purified WT VLPs for a target protein, as assessed using SPR.

### DETAILED DESCRIPTION OF THE INVENTION

### I. Definitions

Unless otherwise defined, all terms of art, notations, and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

The term “about” as used herein refers to the usual error range for the respective value readily known to the skilled person in this technical field. Reference to “about” a value or parameter herein includes (and describes) aspects that are directed to that value or parameter per se.

As used herein, the singular forms “a,” “an,” and “the” include plural referents unless the context clearly dictates otherwise. For example, reference to “an isolated peptide” means one or more isolated peptides.

Throughout this specification and claims, the word “comprise,” or variations such as “comprises” or “comprising” will be understood to imply the inclusion of a stated integer or group of integers but not the exclusion of any other integer or group of integers.

An “effective amount” refers to an amount of an agent (e.g., a therapeutic agent) that is effective to bring about a therapeutic/prophylactic benefit (e.g., as described herein) that is not outweighed by unwanted/undesirable side effects.

The term “pharmaceutical formulation” refers to a preparation which is in such form as to permit the biological activity of the active ingredient or ingredients to be effective, and which contains no additional components which are unacceptably toxic to a subject to which the formulation would be administered. Such formulations are sterile. In one embodiment, the formulation is for intravenous (IV) administration. In another embodiment, the formulation is for subcutaneous (SC) administration.

A “native sequence” protein herein refers to a protein comprising the amino acid sequence of a protein found in nature, including naturally occurring variants of the protein. The term as used herein includes the protein as isolated from a natural source thereof or as recombinantly produced.

The term “protein,” as used herein, refers to any native protein from any source, including viruses (e.g., adeno-associated viruses (AAVs), mammals such as primates (e.g., humans), and rodents (e.g., mice and rats). The term encompasses “full-length,” unprocessed protein any form of the protein that results from processing, e.g., processing in a cell. The term also encompasses naturally occurring variants of the protein, e.g., splice variants or allelic variants, e.g., amino acid substitution mutations or amino acid deletion mutations. The term also includes isolated regions or domains of the protein, e.g., the extracellular domain (ECD).

An “isolated” protein or peptide is one which has been separated from a component of its natural environment. In some aspects, a protein or peptide is purified to greater than 95% or 99% purity as determined by, for example, electrophoresis (e.g., SDS-PAGE, isoelectric focusing (IEF), capillary electrophoresis) or chromatography (e.g., ion exchange or reverse phase HPLC).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The terms “AAV9 major capsid protein VP1” and “AAV9 VP1,” as used herein, refer to any native VP1 from adeno-associated virus serotype 9 (AAV9), unless otherwise indicated. The term encompasses full-length AAV9 VP1 and isolated regions or domains of AAV9 VP1 that comprise at least a portion of the AAV9 VP1 unique N-terminus (i.e., the AAV9 VP1 N-terminal sequence not present in wild-type VP2 or VP3). The term also encompasses naturally occurring variants of AAV9 VP1, e.g., splice variants or allelic variants. The amino acid sequence of an exemplary AAV9 VP1 is provided as SEQ ID NO: 1 and as GenBank accession number AAS99264.1. Minor sequence variations, especially conservative amino acid substitutions of AAV9 VP1 that do not affect AAV9 VP1 function and/or activity, are also contemplated by the invention.

The terms “AAV2 major capsid protein VP1” and “AAV2 VP1,” as used herein, refer to any native VP1 from adeno-associated virus serotype 2 (AAV2), unless otherwise indicated. The term encompasses full-length AAV2 VP1 and isolated regions or domains of AAV2 VP1 that comprise at least a portion of the AAV2 VP1 unique N-terminus (i.e., the AAV2 VP1 N-terminal sequence not present in wild-type VP2 or VP3). The term also encompasses naturally occurring variants of AAV2 VP1, e.g., splice variants or allelic variants. The amino acid sequence of an exemplary AAV2 VP1 is provided as SEQ ID NO: 2 and in NCBI Reference Sequence YP_680426.1. Minor sequence variations, especially conservative amino acid substitutions of AAV2 VP1 that do not affect AAV2 VP1 function and/or activity, are also contemplated by the invention.

The terms “AAV receptor” and “AAVR,” as used herein, refer to any native AAVR from any vertebrate source, including mammals such as primates (e.g., humans) and rodents (e.g., mice and rats), unless otherwise indicated. The term encompasses full-length AAVR and isolated regions or domains thereof. The term also encompasses naturally occurring variants of AAVR, e.g., splice variants or allelic variants. The amino acid sequence of an exemplary AAVR is provided as SEQ ID NO: 3 and as UniProt ID: Q8IZA0. Minor sequence variations, especially conservative amino acid substitutions of AAVR that do not affect AAVR function and/or activity, are also contemplated by the invention.

The term “virus-like particle” or “VLP,” as used herein, refers to an AAV-like particle having a capsid comprising (e.g., consisting essentially of) only one or two of the AAV major capsid protein VP1, AAV minor capsid protein VP2, and AAV minor capsid protein VP3, e.g., comprising only VP1; comprising only VP2; comprising only VP3; comprising only VP1 and VP2; comprising only VP1 and VP3; or comprising only VP2 and VP3.

“Affinity” refers to the strength of the sum total of noncovalent interactions between a single binding site of a molecule (e.g., a receptor) and its binding partner (e.g., a ligand). Unless indicated otherwise, as used herein, “binding affinity” refers to intrinsic binding affinity, which reflects a 1:1 interaction between members of a binding pair (e.g., receptor and ligand). The affinity of a molecule X for its partner Y can generally be represented by the dissociation constant (K_{D}). Affinity can be measured by common methods known in the art, including those described herein.

“Complex” or “complexed” as used herein refers to the association of two or more molecules that interact with each other through bonds and/or forces (e.g., Van der Waals, hydrophobic, hydrophilic forces) that are not peptide bonds. In one aspect, a complex is heteromultimeric. It should be understood that the term “protein complex” or “polypeptide complex” as used herein includes complexes that have a non-protein entity conjugated to a protein in the protein complex (e.g., including, but not limited to, chemical molecules such as a toxin or a detection agent).

The terms “host cell,” “host cell line,” and “host cell culture” are used interchangeably and refer to cells into which exogenous nucleic acid has been introduced, including the progeny of such cells. Host cells include “transfected cells,” “transformed cells,” and “transformants,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. In some aspects, the host cell is stably transformed with the exogenous nucleic acid. In other aspects, the host cell is transiently transformed with the exogenous nucleic acid.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### II. Engineered AAV Capsids

### A. Engineered AAV Capsids Comprising A472H Mutations

In some aspects, the disclosure features an adeno-associated virus (AAV) or virus-like particle (VLP) having a capsid comprising (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation, wherein the amino acid substitution mutations are numbered relative to the AAV9 major capsid protein VP1 (SEQ ID NO: 1).

In some aspects, the AAV or VLP is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV or VLP is AAV1, AAV2, or AAV9. In some aspects, the AAV or VLP is AAV9 or a variant thereof. Further serotypes that may be used in the invention are provided, e.g., in Section II(F), below. A skilled artisan is able to identify corresponding residues in the VP1, VP2, and/or VP3 proteins of two or more AAV serotypes (e.g., is able to determine the residues of a given serotype that correspond to the recited residues of AAV9), e.g., by creating a sequence alignment.

In some aspects, the disclosure features an AAV or VLP having a capsid comprising: (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation; wherein the AAV is AAV9 or a variant thereof and the amino acid substitution mutations are numbered relative to the AAV9 major capsid protein VP1 (SEQ ID NO: 1).

Combination of A472H with A273N, Q387K, and/or E500R Mutations

In some aspects, (a) the major capsid protein VP1 further comprises an A273N amino acid substitution mutation; (b) the minor capsid protein VP2 further comprises an A273N amino acid substitution mutation; and/or (c) the minor capsid protein VP3 further comprises an A273N amino acid substitution mutation, i.e., VP1, VP2, and/or VP3 comprise A273N and A472H amino acid substitution mutations.

In some aspects, (a) the major capsid protein VP1 further comprises a Q387K amino acid substitution mutation; (b) the minor capsid protein VP2 further comprises a Q387K amino acid substitution mutation; and/or (c) the minor capsid protein VP3 further comprises a Q387K amino acid substitution mutation, i.e., VP1, VP2, and/or VP3 comprise Q387K and A472H amino acid substitution mutations.

In some aspects, (a) the major capsid protein VP1 further comprises an E500R amino acid substitution mutation; (b) the minor capsid protein VP2 further comprises an E500R amino acid substitution mutation; and/or (c) the minor capsid protein VP3 further comprises an E500R amino acid substitution mutation, i.e., VP1, VP2, and/or VP3 comprise E500R and A472H amino acid substitution mutations.

In some aspects, (a) the major capsid protein VP1 further comprises A273N, Q387K, and E500R amino acid substitution mutations; (b) the minor capsid protein VP2 further comprises A273N, Q387K, and E500R amino acid substitution mutations; and/or (c) the minor capsid protein VP3 further comprises A273N, Q387K, and E500R amino acid substitution mutations, i.e., VP1, VP2, and/or VP3 comprise A273N, Q387K, A472H, and E500R amino acid substitution mutations.

In some aspects, the disclosure features an AAV or VLP having a capsid comprising: (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; wherein the amino acid substitution mutations are numbered relative to the AAV9 major capsid protein VP1 (SEQ ID NO: 1).

In some aspects, the disclosure features an AAV or VLP having a capsid comprising: (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; wherein the AAV is AAV9 or a variant thereof and the amino acid substitution mutations are numbered relative to the AAV9 major capsid protein VP1 (SEQ ID NO: 1).

In some aspects, the modified AAV or VLP has an increased affinity for AAV receptor (AAVR) relative to a control AAV or VLP having a capsid that does not comprise any of the indicated amino acid substitution mutations (e.g., an AAV or VLP of the same serotype that does not comprise any of the indicated amino acid substitution mutations). In some aspects in which the modified AAV or VLP comprises two of the four A273N, Q387K, A472H, and E500R amino acid substitution mutations, the modified AAV or VLP has an increased affinity for AAVR relative to a control AAV or VLP having a capsid that comprises only one of the indicated amino acid substitution mutations. In some aspects in which the modified AAV or VLP comprises three of the four A273N, Q387K, A472H, and E500R amino acid substitution mutations, the modified AAV or VLP has an increased affinity for AAVR relative to a control AAV or VLP having a capsid that comprises only one or only two of the indicated amino acid substitution mutations. In some aspects in which the modified AAV or VLP comprises all four of the A273N, Q387K, A472H, and E500R amino acid substitution mutations, the modified AAV or VLP has an increased affinity for AAVR relative to a control AAV or VLP having a capsid that comprises only one, only two, or only three of the indicated amino acid substitution mutations.

In some aspects, the modified AAV or VLP and has an increased affinity for AAVR relative to a control AAV or VLP derivative thereof, e.g., an AAV or a VLP derivative thereof having a capsid that (a) does not comprise any of the indicated amino acid substitution mutations or (b) comprises fewer of the indicated amino acid substitution mutations than the modified AAV or VLP of interest. For example, in some aspects, affinity of the modified AAV or VLP for the AAVR is increased by at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, or more than 200%, e.g., is improved by 5%-10%, 10%-20%, 20%-30%, 30%-40%, 40%-50%, 50%-60%, 60%-70%, 70%-80%, 80%-90%, 90%-100%, 100%-150%, or 150%-200% relative to the affinity of a control AAV or VLP. In some aspects, the modified AAV or VLP is AAV9 or a variant thereof.

Combination of A472H with T592D and/or A593E Mutations

In some aspects, (a) the major capsid protein VP1 further comprises a T592D amino acid substitution mutation; (b) the minor capsid protein VP2 further comprises a T592D amino acid substitution mutation; and/or (c) the minor capsid protein VP3 further comprises a T592D amino acid substitution mutation, i.e., VP1, VP2, and/or VP3 comprise A472H and T592D amino acid substitution mutations, wherein the A472H amino acid substitution mutation is numbered relative to the AAV9 major capsid protein VP1 (SEQ ID NO: 1) and the T592D amino acid substitution mutation is numbered relative to the AAV2 major capsid protein VP1 (SEQ ID NO: 2).

In some aspects, (a) the major capsid protein VP1 further comprises an A593E amino acid substitution mutation; (b) the minor capsid protein VP2 further comprises an A593E amino acid substitution mutation; and/or (c) the minor capsid protein VP3 further comprises an A593E amino acid substitution mutation, i.e., VP1, VP2, and/or VP3 comprise A472H and A593E amino acid substitution mutations, wherein the A472H amino acid substitution mutation is numbered relative to the AAV9 major capsid protein VP1 (SEQ ID NO: 1) and the A593E amino acid substitution mutation is numbered relative to the AAV2 major capsid protein VP1 (SEQ ID NO: 2).

In some aspects, (a) the major capsid protein VP1 further comprises T592D and A593E amino acid substitution mutations; (b) the minor capsid protein VP2 further comprises T592D and A593E amino acid substitution mutations; and/or (c) the minor capsid protein VP3 further comprises T592D and A593E amino acid substitution mutations, i.e., VP1, VP2, and/or VP3 comprise A472H, T592D, and A593E amino acid substitution mutations, wherein the A472H amino acid substitution mutation is numbered relative to the AAV9 major capsid protein VP1 (SEQ ID NO: 1) and the T592D and A593E amino acid substitution mutations are numbered relative to the AAV2 major capsid protein VP1 (SEQ ID NO: 2).

In some aspects, the modified AAV or VLP has an increased affinity for AAV receptor (AAVR) relative to a control AAV or VLP having a capsid that does not comprise any of the indicated amino acid substitution mutations (e.g., an AAV or VLP of the same serotype that does not comprise any of the indicated amino acid substitution mutations).

In some aspects, the modified AAV or VLP and has an increased affinity for AAVR relative to a control AAV or VLP derivative thereof, e.g., an AAV or a VLP derivative thereof having a capsid that (a) does not comprise any of the indicated amino acid substitution mutations or (b) comprises fewer of the indicated amino acid substitution mutations than the modified AAV or VLP of interest. For example, in some aspects, affinity of the modified AAV or VLP for the AAVR is increased by at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, or more than 200%, e.g., is improved by 5%-10%, 10%-20%, 20%-30%, 30%-40%, 40%-50%, 50%-60%, 60%-70%, 70%-80%, 80%-90%, 90%-100%, 100%-150%, or 150%-200% relative to the affinity of a control AAV or VLP.

Combination of A472H with E500P Mutation

In some aspects, (a) the major capsid protein VP1 further comprises an E500P amino acid substitution mutation; (b) the minor capsid protein VP2 further comprises an E500P amino acid substitution mutation; and/or (c) the minor capsid protein VP3 further comprises an E500P amino acid substitution mutation, i.e., VP1, VP2, and/or VP3 comprise A472H and E500P amino acid substitution mutations, wherein the A472H and E500P amino acid substitution mutations are numbered relative to the AAV9 major capsid protein VP1 (SEQ ID NO: 1).

In some aspects, the disclosure features an AAV having a capsid comprising (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations, wherein the amino acid substitution mutations are numbered relative to the AAV9 major capsid protein VP1 (SEQ ID NO: 1).

In some aspects, the modified AAV or VLP has an increased affinity for AAV receptor (AAVR) relative to a control AAV or VLP having a capsid that does not comprise any of the indicated amino acid substitution mutations (e.g., an AAV or VLP of the same serotype that does not comprise any of the indicated amino acid substitution mutations).

In some aspects, the modified AAV or VLP and has an increased affinity for AAVR relative to a control AAV or VLP derivative thereof, e.g., an AAV or a VLP derivative thereof having a capsid that (a) does not comprise any of the indicated amino acid substitution mutations or (b) comprises fewer of the indicated amino acid substitution mutations than the modified AAV or VLP of interest. For example, in some aspects, affinity of the modified AAV or VLP for the AAVR is increased by at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, or more than 200%, e.g., is improved by 5%-10%, 10%-20%, 20%-30%, 30%-40%, 40%-50%, 50%-60%, 60%-70%, 70%-80%, 80%-90%, 90%-100%, 100%-150%, or 150%-200% relative to the affinity of a control AAV or VLP.

In some aspects, the modified AAV or VLP is AAV2 or a variant thereof.

In some aspects, the yield and/or titer in an expression system (e.g., EXPI293™ cells) of any of the modified AAVs or VLPs provided herein, e.g., AAVs or VLPs comprising one or more of the A273N, Q387K, A472H, E500R, T592D, and A593E amino acid substitution mutations, is not substantially reduced relative to the yield and/or titer of wild-type AAVs or VLPs in the expression system (e.g., is similar to, equal to, or greater than the yield and/or titer of wild-type AAVs or VLPs in the expression system).

### Methods of Improving Affinity for AAVR

In some aspects, the disclosure features a method of improving the affinity of an AAV for AAV receptor (AAVR), the method comprising providing any of the above-described modified AAVs (e.g., contacting a cell with any of the above-described modified AAVs, wherein the contacting results in increased affinity for AAVR).

For example, in some aspects, the disclosure features a method of improving the affinity of an AAV for AAV receptor (AAVR), the method comprising providing a modified AAV having a capsid comprising (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation, wherein the amino acid substitution mutations are numbered relative to the AAV9 major capsid protein VP1 (SEQ ID NO: 1).

In some aspects, the disclosure features a method of improving the affinity of an AAV for AAV receptor (AAVR), the method comprising providing a modified AAV having a capsid comprising (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations, wherein the amino acid substitution mutations are numbered relative to the AAV9 major capsid protein VP1 (SEQ ID NO: 1).

In some aspects, the disclosure features a method of improving the affinity of an AAV for AAV receptor (AAVR), the method comprising providing a modified AAV having a capsid comprising (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations, wherein the amino acid substitution mutations are numbered relative to the AAV9 major capsid protein VP1 (SEQ ID NO: 1).

For example, in some aspects, affinity of the modified AAV or VLP for the AAVR is increased by at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, or more than 200%, e.g., is improved by 5%-10%, 10%-20%, 20%-30%, 30%-40%, 40%-50%, 50%-60%, 60%-70%, 70%-80%, 80%-90%, 90%-100%, 100%-150%, or 150%-200% relative to the affinity of a control AAV or VLP.

Alternative amino acid substitutions at position A472 In some embodiments of any of the aspects provided herein, the modified AAV or VLP may comprise an A472F, A472K, A472N, A472W, or A472Y amino acid substitution mutation instead of the A472H amino acid substitution mutation.

For example, in some aspects, the disclosure features an AAV or VLP having a capsid comprising (a) a major capsid protein VP1 comprising an A472F, A472K, A472N, A472W, or A472Y amino acid substitution mutation; (b) a minor capsid protein VP2 comprising an A472F, A472K, A472N, A472W, or A472Y amino acid substitution mutation; and/or (c) a minor capsid protein VP3 comprising an A472F, A472K, A472N, A472W, or A472Y amino acid substitution mutation, wherein the amino acid substitution mutations are numbered relative to the AAV9 major capsid protein VP1 (SEQ ID NO: 1).

The AAV or VLP comprising the A472F, A472K, A472N, A472W, or A472Y amino acid substitution mutation may further comprise one or more additional amino acid substitutions provided herein, e.g., may comprise a major capsid protein VP1, a minor capsid protein VP2, and/or a minor capsid protein VP3 that comprises one, two, or all three of an A273N amino acid substitution mutation, a Q387K amino acid substitution mutation, and an E500R amino acid substitution mutation, wherein the amino acid substitution mutations are numbered relative to the AAV9 major capsid protein VP1 (SEQ ID NO: 1).

Additionally or alternatively, the AAV or VLP comprising the A472F, A472K, A472N, A472W, or A472Y amino acid substitution mutation may further comprise a major capsid protein VP1, a minor capsid protein VP2, and/or a minor capsid protein VP3 that comprises one or both of a T592D amino acid substitution mutation and an A593E amino acid substitution mutation, wherein the A472H amino acid substitution mutation is numbered relative to the AAV9 major capsid protein VP1 (SEQ ID NO: 1) and the T592D and A593E amino acid substitution mutations are numbered relative to the AAV2 major capsid protein VP1 (SEQ ID NO: 2).

Additionally or alternatively, the AAV or VLP comprising the A472F, A472K, A472N, A472W, or A472Y amino acid substitution mutation may further comprise a major capsid protein VP1, a minor capsid protein VP2, and/or a minor capsid protein VP3 that comprises an E500P amino acid substitution mutation, wherein the E500P amino acid substitution mutation is numbered relative to the AAV9 major capsid protein VP1 (SEQ ID NO: 1).

### B. Engineered AAV Capsids Comprising E500R Mutations

In some aspects, the disclosure features an adeno-associated virus (AAV) or virus-like particle (VLP) having a capsid comprising (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation, wherein the amino acid substitution mutations are numbered relative to the AAV9 major capsid protein VP1 (SEQ ID NO: 1).

In some aspects, the AAV or VLP is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV or VLP is AAV1, AAV2, or AAV9. Further serotypes that may be used in the invention are provided, e.g., in Section II(F), below. A skilled artisan is able to identify corresponding residues in the VP1, VP2, and/or VP3 proteins of two or more AAV serotypes (e.g., is able to determine the residues of a given serotype that correspond to the recited residues of AAV9), e.g., by creating a sequence alignment.

In some aspects, the disclosure features an AAV or VLP having a capsid comprising: (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; wherein the AAV is AAV9 or a variant thereof and the amino acid substitution mutations are numbered relative to the AAV9 major capsid protein VP1 (SEQ ID NO: 1).

In some aspects, (a) the major capsid protein VP1 further comprises an A273N amino acid substitution mutation; (b) the minor capsid protein VP2 further comprises an A273N amino acid substitution mutation; and/or (c) the minor capsid protein VP3 further comprises an A273N amino acid substitution mutation, i.e., VP1, VP2, and/or VP3 comprise A273N and E500R amino acid substitution mutations.

In some aspects, (a) the major capsid protein VP1 further comprises a Q387K amino acid substitution mutation; (b) the minor capsid protein VP2 further comprises a Q387K amino acid substitution mutation; and/or (c) the minor capsid protein VP3 further comprises a Q387K amino acid substitution mutation, i.e., VP1, VP2, and/or VP3 comprise Q387K and E500R amino acid substitution mutations.

In some aspects, (a) the major capsid protein VP1 further comprises an A472H amino acid substitution mutation; (b) the minor capsid protein VP2 further comprises an A472H amino acid substitution mutation; and/or (c) the minor capsid protein VP3 further comprises an A472H amino acid substitution mutation, i.e., VP1, VP2, and/or VP3 comprise A472H and E500R amino acid substitution mutations.

In some aspects, (a) the major capsid protein VP1 further comprises A273N and Q387K amino acid substitution mutations; (b) the minor capsid protein VP2 further comprises A273N and Q387K amino acid substitution mutations; and/or (c) the minor capsid protein VP3 further comprises A273N and Q387K amino acid substitution mutations, i.e., VP1, VP2, and/or VP3 comprise A273N, Q387K, and E500R amino acid substitution mutations.

In some aspects, (a) the major capsid protein VP1 further comprises A273N, Q387K, and A472H amino acid substitution mutations; (b) the minor capsid protein VP2 further comprises A273N, Q387K, and A472H amino acid substitution mutations; and/or (c) the minor capsid protein VP3 further comprises A273N, Q387K, and A472H amino acid substitution mutations, i.e., VP1, VP2, and/or VP3 comprise A273N, Q387K, A472H, and E500R amino acid substitution mutations.

In some aspects, the disclosure features an AAV or VLP having a capsid comprising: (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; wherein the amino acid substitution mutations are numbered relative to the AAV9 major capsid protein VP1 (SEQ ID NO: 1).

In some aspects, the disclosure features an AAV or VLP having a capsid comprising: (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations; wherein the AAV is AAV9 or a variant thereof and the amino acid substitution mutations are numbered relative to the AAV9 major capsid protein VP1 (SEQ ID NO: 1).

In some aspects, (a) the major capsid protein VP1 further comprises an A472H amino acid substitution mutation; (b) the minor capsid protein VP2 further comprises an A472H amino acid substitution mutation; and/or (c) the minor capsid protein VP3 further comprises an A472H amino acid substitution mutation. In some aspects, (a) the major capsid protein VP1 further comprises an A472H amino acid substitution mutation; (b) the minor capsid protein VP2 further comprises an A472H amino acid substitution mutation; and/or (c) the minor capsid protein VP3 further comprises an A472H amino acid substitution mutation, i.e., VP1, VP2, and/or VP3 comprise A472H and E500R amino acid substitution mutations.

In some aspects, the modified AAV or VLP has an increased affinity for AAV receptor (AAVR) relative to a control AAV or VLP having a capsid that does not comprise any of the indicated amino acid substitution mutations (e.g., an AAV or VLP of the same serotype that does not comprise any of the indicated amino acid substitution mutations). In some aspects in which the modified AAV or VLP comprises two of the three A273N, Q387K, and E500R amino acid substitution mutations, the modified AAV or VLP has an increased affinity for AAVR relative to a control AAV or VLP having a capsid that comprises only one of the indicated amino acid substitution mutations. In some aspects in which the modified AAV or VLP comprises all three of the A273N, Q387K, and E500R amino acid substitution mutations, the modified AAV or VLP has an increased affinity for AAVR relative to a control AAV or VLP having a capsid that comprises only one or only two of the indicated amino acid substitution mutations.

In some aspects, the modified AAV or VLP is AAV9 or a variant thereof and has an increased affinity for AAVR relative to a control AAV or VLP derivative thereof, e.g., an AAV9 or a VLP derivative thereof having a capsid that (a) does not comprise any of the indicated amino acid substitution mutations or (b) comprises fewer of the indicated amino acid substitution mutations than the modified AAV or VLP of interest. For example, in some aspects, affinity of the modified AAV or VLP for the AAVR is increased by at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, or more than 200%, e.g., is improved by 5%-10%, 10%-20%, 20%-30%, 30%-40%, 40%-50%, 50%-60%, 60%-70%, 70%-80%, 80%-90%, 90%-100%, 100%-150%, or 150%-200% relative to the affinity of a control AAV or VLP.

### Methods of Improving Affinity for AAVR

In some aspects, the disclosure features a method of improving the affinity of an AAV for AAV receptor (AAVR), the method comprising providing any of the above-described modified AAVs (e.g., contacting a cell with any of the above-described modified AAVs, wherein the contacting results in increased affinity for AAVR).

For example, in some aspects, the disclosure features a method of improving the affinity of an AAV for AAV receptor (AAVR), the method comprising providing a modified AAV having a capsid comprising (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation, wherein the amino acid substitution mutations are numbered relative to the AAV9 major capsid protein VP1 (SEQ ID NO: 1).

In some aspects, the disclosure features a method of improving the affinity of an AAV for AAV receptor (AAVR), the method comprising providing a modified AAV having a capsid comprising (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations, wherein the amino acid substitution mutations are numbered relative to the AAV9 major capsid protein VP1 (SEQ ID NO: 1).

For example, in some aspects, affinity of the modified AAV or VLP for the AAVR is increased by at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, or more than 200%, e.g., is improved by 5%-10%, 10%-20%, 20%-30%, 30%-40%, 40%-50%, 50%-60%, 60%-70%, 70%-80%, 80%-90%, 90%-100%, 100%-150%, or 150%-200% relative to the affinity of a control AAV or VLP.

### Alternative Amino Acid Substitution at Position E500

In some embodiments of any of the aspects provided herein, the modified AAV or VLP may comprise an E500P amino acid substitution mutation instead of the E500R amino acid substitution mutation.

For example, in some aspects, the disclosure features an AAV or VLP having a capsid comprising (a) a major capsid protein VP1 comprising an E500P amino acid substitution mutation; (b) a minor capsid protein VP2 comprising an E500P amino acid substitution mutation; and/or (c) a minor capsid protein VP3 comprising an E500P amino acid substitution mutation, wherein the amino acid substitution mutations are numbered relative to the AAV9 major capsid protein VP1 (SEQ ID NO: 1).

The AAV or VLP comprising the E500P amino acid substitution mutation may further comprise one or more additional amino acid substitutions provided herein, e.g., may comprise a major capsid protein VP1, a minor capsid protein VP2, and/or a minor capsid protein VP3 that comprises one, two, or all three of an A273N amino acid substitution mutation, a Q387K amino acid substitution mutation, and an A472H amino acid substitution mutation, wherein the amino acid substitution mutations are numbered relative to the AAV9 major capsid protein VP1 (SEQ ID NO: 1).

### C. Engineered AAV Capsids Comprising T592D and A593E Mutations

In some aspects, the disclosure features an AAV or VLP having a capsid comprising: (a) a major capsid protein VP1 comprising T592D and/or A593E amino acid substitution mutations; (b) a minor capsid protein VP2 comprising T592D and/or A593E amino acid substitution mutations; and/or (c) a minor capsid protein VP3 comprising T592D and/or A593E amino acid substitution mutations; wherein the amino acid substitution mutations are numbered relative to the AAV2 major capsid protein VP1 (SEQ ID NO: 2).

In some aspects, the disclosure features an AAV or VLP having a capsid comprising: (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations; wherein the amino acid substitution mutations are numbered relative to the AAV2 major capsid protein VP1 (SEQ ID NO: 2).

In some aspects, the AAV or VLP is of a serotype that interacts with AAVR (e.g., AAV1, AAV2, AAV3, AAV6, AAV7, AAV8, AAV9, AAV10, AAV11, or AAV12). In some aspects, the AAV or VLP is AAV1, AAV2, or AAV9. Further serotypes that may be used in the invention are provided, e.g., in Section II(F), below.

In some aspects, the disclosure features an AAV or VLP having a capsid comprising: (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations; wherein the AAV is AAV2 or a variant thereof and wherein the amino acid substitution mutations are numbered relative to the AAV2 major capsid protein VP1 (SEQ ID NO: 2).

In some aspects, the modified AAV or VLP has an increased affinity for AAV receptor (AAVR) relative to a control AAV or VLP having a capsid that does not comprise either of the indicated amino acid substitution mutations or that comprises only one of the two indicated amino acid substitution mutations (e.g., an AAV or VLP of the same serotype that does not comprise either of the indicated amino acid substitution mutations or that comprises only one of the two indicated amino acid substitution mutations).

In some aspects, the modified AAV or VLP is AAV2 or a variant thereof and has an increased affinity for AAVR relative to a control AAV2 or a VLP derivative thereof, e.g., an AAV2 or a VLP derivative thereof having a capsid that (a) does not comprise either of the indicated amino acid substitution mutations or (b) comprises only one of the indicated amino acid substitution mutations. For example, in some aspects, affinity of the modified AAV or VLP for the AAVR is increased by at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, or more than 200%, e.g., is improved by 5%-10%, 10%-20%, 20%-30%, 30%-40%, 40%-50%, 50%-60%, 60%-70%, 70%-80%, 80%-90%, 90%-100%, 100%-150%, or 150%-200% relative to the affinity of a control AAV or VLP.

### Methods of Improving Affinity for AAVR

In some aspects, the disclosure features a method of improving the affinity of an AAV for AAV receptor (AAVR), the method comprising providing any of the above-described modified AAVs (e.g., contacting a cell with any of the above-described modified AAVs, wherein the contacting results in increased affinity for AAVR).

For example, in some aspects, the disclosure features a method of improving the affinity of an AAV for AAV receptor (AAVR), the method comprising providing a modified AAV having a capsid comprising (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations, wherein the AAV is AAV2 or a variant thereof and the amino acid substitution mutations are numbered relative to the AAV2 major capsid protein VP1 (SEQ ID NO: 2).

For example, in some aspects, affinity of the modified AAV or VLP for the AAVR is increased by at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, or more than 200%, e.g., is improved by 5%-10%, 10%-20%, 20%-30%, 30%-40%, 40%-50%, 50%-60%, 60%-70%, 70%-80%, 80%-90%, 90%-100%, 100%-150%, or 150%-200% relative to the affinity of a control AAV or VLP.

### D. Engineered AAV Capsids Comprising I451Y, S454I, and P504I Mutations

In some aspects, the disclosure features an adeno-associated virus (AAV) or virus-like particle (VLP) having a capsid comprising (a) a major capsid protein VP1 comprising one, two, or all three of an I451Y, I451T, or I451M amino acid substitution mutation, a S454I or S454F amino acid substitution mutation, and a P504I, P504T, or P504V amino acid substitution mutation; (b) a minor capsid protein VP2 comprising one, two, or all three of an I451Y, I451T, or I451M amino acid substitution mutation, a S454I or S454F amino acid substitution mutation, and a P504I, P504T, or P504V amino acid substitution mutation; and/or (c) a minor capsid protein VP3 comprising one, two, or all three of an I451Y, I451T, or I451M amino acid substitution mutation, a S454I or S454F amino acid substitution mutation, and a P504I, P504T, or P504V amino acid substitution mutation, wherein the amino acid substitution mutations are numbered relative to the AAV9 major capsid protein VP1 (SEQ ID NO: 1).

In some aspects, the disclosure features an adeno-associated virus (AAV) or virus-like particle (VLP) having a capsid comprising (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations, wherein the amino acid substitution mutations are numbered relative to the AAV9 major capsid protein VP1 (SEQ ID NO: 1).

In some aspects, the AAV or VLP is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV or VLP is AAV1, AAV2, or AAV9. Further serotypes that may be used in the invention are provided, e.g., in Section II(F), below. A skilled artisan is able to identify corresponding residues in the VP1, VP2, and/or VP3 proteins of two or more AAV serotypes (e.g., is able to determine the residues of a given serotype that correspond to the recited residues of AAV9), e.g., by creating a sequence alignment.

In some aspects, the disclosure features an AAV or VLP having a capsid comprising: (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations; wherein the AAV is AAV9 or a variant thereof and the amino acid substitution mutations are numbered relative to the AAV9 major capsid protein VP1 (SEQ ID NO: 1).

In some aspects, (a) the major capsid protein VP1 further comprises an E500P amino acid substitution mutation; (b) the minor capsid protein VP2 further comprises an E500P amino acid substitution mutation; and/or (c) the minor capsid protein VP3 further comprises an E500P amino acid substitution mutation, i.e., VP1, VP2, and/or VP3 comprise I451Y, S454I, P504I, and E500P amino acid substitution mutations.

In some aspects, (a) the major capsid protein VP1 further comprises an E500R amino acid substitution mutation; (b) the minor capsid protein VP2 further comprises an E500R amino acid substitution mutation; and/or (c) the minor capsid protein VP3 further comprises an E500R amino acid substitution mutation, i.e., VP1, VP2, and/or VP3 comprise I451Y, S454I, P504I, and E500R amino acid substitution mutations.

In some aspects, the disclosure features an AAV or VLP having a capsid comprising: (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; wherein the amino acid substitution mutations are numbered relative to the AAV9 major capsid protein VP1 (SEQ ID NO: 1).

In some aspects, the disclosure features an AAV or VLP having a capsid comprising: (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; wherein the AAV is AAV9 or a variant thereof and the amino acid substitution mutations are numbered relative to the AAV9 major capsid protein VP1 (SEQ ID NO: 1).

In some aspects, the disclosure features an AAV or VLP having a capsid comprising: (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations; wherein the amino acid substitution mutations are numbered relative to the AAV9 major capsid protein VP1 (SEQ ID NO: 1).

In some aspects, the disclosure features an AAV or VLP having a capsid comprising: (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations; wherein the AAV is AAV9 or a variant thereof and the amino acid substitution mutations are numbered relative to the AAV9 major capsid protein VP1 (SEQ ID NO: 1).

In some aspects, the modified AAV or VLP has an increased affinity for AAV receptor (AAVR) relative to a control AAV or VLP having a capsid that does not comprise any of the indicated amino acid substitution mutations (e.g., an AAV or VLP of the same serotype that does not comprise any of the indicated amino acid substitution mutations). In some aspects in which the modified AAV or VLP comprises three of the four I451Y, S454I, E500P, and P504I amino acid substitution mutations or three of the four I451Y, S454I, E500R, and P504I amino acid substitution mutations, the modified AAV or VLP has an increased affinity for AAVR relative to a control AAV or VLP having a capsid that comprises only one or only two of the indicated amino acid substitution mutations. In some aspects in which the modified AAV or VLP comprises all four of the I451Y, S454I, E500P, and P504I amino acid substitution mutations or all four of the I451Y, S454I, E500R, and P504I amino acid substitution mutations, the modified AAV or VLP has an increased affinity for AAVR relative to a control AAV or VLP having a capsid that comprises only one, only two, or only three of the indicated amino acid substitution mutations.

In some aspects, the modified AAV or VLP is AAV9 or a variant thereof and has an increased affinity for AAVR relative to a control AAV or VLP derivative thereof, e.g., an AAV9 or a VLP derivative thereof having a capsid that (a) does not comprise any of the indicated amino acid substitution mutations or (b) comprises fewer of the indicated amino acid substitution mutations than the modified AAV or VLP of interest. For example, in some aspects, affinity of the modified AAV or VLP for the AAVR is increased by at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, or more than 200%, e.g., is improved by 5%-10%, 10%-20%, 20%-30%, 30%-40%, 40%-50%, 50%-60%, 60%-70%, 70%-80%, 80%-90%, 90%-100%, 100%-150%, or 150%-200% relative to the affinity of a control AAV or VLP.

### Methods of Improving Affinity for AAVR

In some aspects, the disclosure features a method of improving the affinity of an AAV for AAV receptor (AAVR), the method comprising providing any of the above-described modified AAVs (e.g., contacting a cell with any of the above-described modified AAVs, wherein the contacting results in increased affinity for AAVR).

For example, in some aspects, the disclosure features a method of improving the affinity of an AAV for AAV receptor (AAVR), the method comprising providing a modified AAV having a capsid comprising (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations, wherein the amino acid substitution mutations are numbered relative to the AAV9 major capsid protein VP1 (SEQ ID NO: 1).

In some aspects, the disclosure features a method of improving the affinity of an AAV for AAV receptor (AAVR), the method comprising providing a modified AAV having a capsid comprising (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations, wherein the amino acid substitution mutations are numbered relative to the AAV9 major capsid protein VP1 (SEQ ID NO: 1).

In some aspects, the disclosure features a method of improving the affinity of an AAV for AAV receptor (AAVR), the method comprising providing a modified AAV having a capsid comprising (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations, wherein the AAV is AAV9 or a variant thereof and the amino acid substitution mutations are numbered relative to the AAV9 major capsid protein VP1 (SEQ ID NO: 1), wherein the amino acid substitution mutations are numbered relative to the AAV9 major capsid protein VP1 (SEQ ID NO: 1).

For example, in some aspects, affinity of the modified AAV or VLP for the AAVR is increased by at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, or more than 200%, e.g., is improved by 5%-10%, 10%-20%, 20%-30%, 30%-40%, 40%-50%, 50%-60%, 60%-70%, 70%-80%, 80%-90%, 90%-100%, 100%-150%, or 150%-200% relative to the affinity of a control AAV or VLP.

### Alternative Amino Acid Substitutions at Position I451

In some embodiments of any of the aspects provided herein, the modified AAV or VLP may comprise an I451Y or I451M amino acid substitution mutation instead of the I451T amino acid substitution mutation.

### Alternative Amino Acid Substitution at Position S454

In some embodiments of any of the aspects provided herein, the modified AAV or VLP may comprise a S454F amino acid substitution mutation instead of the S454I amino acid substitution mutation.

### Alternative Amino Acid Substitutions at Position P504

In some embodiments of any of the aspects provided herein, the modified AAV or VLP may comprise a P504T or P504V amino acid substitution mutation instead of the P504I amino acid substitution mutation.

### E. Further Capsid Mutations

In some aspects, the disclosure features an adeno-associated virus (AAV) or virus-like particle (VLP) having a capsid comprising (a) a major capsid protein VP1; (b) a minor capsid protein VP2; and/or (c) a minor capsid protein VP3 comprising one or more of an I451T, I451Y, or I451M amino acid substitution mutation; an S454I or S454F amino acid substitution mutation; an A472F, A472H, A472K, A472N, A472W, or A472Y amino acid substitution mutation; a P504I, P504T, or P504V amino acid substitution mutation; an E500P or E500R amino acid substitution mutation; an A273N amino acid substitution mutation; a Q387K amino acid substitution mutation; an S263A or S263Y amino acid substitution mutation, and a W503H amino acid substitution mutation, wherein the amino acid substitution mutations are numbered relative to the AAV9 major capsid protein VP1 (SEQ ID NO: 1). In some aspects, the AAV or VLP has improved transduction affinity, has increased affinity for AAVR, has an increased rate of delivery to the nucleus of a target cell, and/or transduces a target cell at a lower dose than a control AAV or VLP, e.g., an AAV or VLP that does not comprise any of the indicated amino acid substitution mutations.

In some aspects, the AAV or VLP comprises two, three, four, five, six, seven, eight, or nine of the listed amino acid substitution mutations. For example, in some aspects, the disclosure features an AAV or VLP having a capsid comprising (a) a major capsid protein VP1; (b) a minor capsid protein VP2; and/or (c) a minor capsid protein VP3 comprising I451T and A472H, I451Y and A472H, S454I and A472H, A472H and P504I, A472H and P504T, A472H and E500R, A273N and A472H, A472H and E500P, Q387K and A472H, A273N and Q387K, A273N and E500R, or Q387K and E500R amino acid substitution mutations. In some aspects, the disclosure features an AAV or VLP having a capsid comprising (a) a major capsid protein VP1; (b) a minor capsid protein VP2; and/or (c) a minor capsid protein VP3 comprising Q387K, A472H, and E500P; A273N, A472H, and E500P; I451Y, S454I, and E500P; or A273N, Q387K, and E500R amino acid substitution mutations. In some aspects, the disclosure features an AAV or VLP having a capsid comprising (a) a major capsid protein VP1; (b) a minor capsid protein VP2; and/or (c) a minor capsid protein VP3 comprising I451Y, S454I, E500P, and P504I; I451Y, S454I, E500P, and P504T; or A273N, Q387K, A472H, and E500P amino acid substitution mutations.

In some aspects, the AAV or VLP is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV or VLP is AAV1, AAV2, or AAV9. In some aspects, the AAV or VLP is AAV9 or a variant thereof. Further serotypes that may be used in the invention are provided, e.g., in Section II(F), below. A skilled artisan is able to identify corresponding residues in the VP1, VP2, and/or VP3 proteins of two or more AAV serotypes (e.g., is able to determine the residues of a given serotype that correspond to the recited residues of AAV9), e.g., by creating a sequence alignment.

### F. Exemplary AAV Serotypes

As used herein, the serotype of an AAV or virus-like particle (VLP) refers to the serotype of the capsid protein(s) of the AAV or VLP.

An AAV or VLP of the disclosure may be, e.g., of a natural AAV serotype (e.g., AAV1, AAV2, AAV3, AAV3B, AAV4, AAV5, AAV6, AAV7, AAV8, AAV9, AAV10, AAV11, or AAV12), an engineered AAV serotype, or any variant, derivative, or pseudotype thereof. For example, the AAV may be a self-complementary AAV (scAAV), a chimeric AAV, a hybrid AAV, AAVrh74, AAVanc80L65, AAVrh.10, AAVrh.74, AAV2/1, AAV2/5, AAV2/6, AAV2/8, AAV2/9, an AAV2-AAV3 hybrid, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6 (Y445F/Y73 IF), AAV2.5T, AAV-HAE1/2, AAV clone 32/83, AAVShHIO, AAV2 (Y->F), AAV8 (Y733F), AAV2.15, AAV2.4, AAVM41, or AAVr3.45. AAV serotypes that may be used in the invention are described, e.g., in Asokan et al., Mol Ther., 20(4): 699-708, 2012.

### G. AAVs and VLPs

In some aspects, the disclosure provides an adeno-associated virus (AAV) having a capsid comprising all three of major capsid protein VP1, minor capsid protein VP2, and minor capsid protein VP3. In some aspects, all three of VP1, VP2, and VP3 comprise the specified amino acid substitution mutation(s) (e.g., all three of VP1, VP2, and VP3 comprise A273N, Q387K, A472H, and/or E500R amino acid substitution mutations or all three of VP1, VP2, and VP3 comprise T592D and A593E amino acid substitution mutations). In other aspects, only a subset of VP1, VP2, and VP3 comprise the indicated amino acid substitution mutation(s) (e.g., only VP1; only VP2; only VP3; only VP1 and VP2; only VP1 and VP3; or only VP2 and VP3 comprise the mutation(s)).

Provided herein are AAVs having a capsid comprising (a) a major capsid protein VP1 comprising one or more amino acid substitution mutations (“mutant VP1”); (b) a minor capsid protein VP2 comprising one or more amino acid substitution mutations (“mutant VP2”); and/or (c) a minor capsid protein VP3 comprising one or more amino acid substitution mutations (“mutant VP3”) (e.g., amino acid substitution mutations as described in Sections IIA-IIE, above). Accordingly, an AAV provided herein may comprise (i) a mutant VP1, a wild-type VP2, and a wild-type VP3; (ii) a wild-type VP1, a mutant VP2, and a wild-type VP3; (iii) a wild-type VP1, a wild-type VP2, and a mutant VP3; (iv) a mutant VP1, a mutant VP2, and a wild-type VP3; (v) a mutant VP1, a wild-type VP2, and a mutant VP3; or (vi) a wild-type VP1, a mutant VP2, and a mutant VP3 (i.e., may be a chimera), or may comprise a mutant VP1, a mutant VP2, and a mutant VP3.

In some aspects in which the one or more amino acid substitution mutations increase the affinity of the AAV subunit (VP1, VP2, or VP3) for AAVR, a chimeric AAV (e.g., an AAV comprising amino acid substitution mutations in only one or only two subunits) has lower affinity for AAVR than an AAV that comprises the amino acid substitution mutations in all three subunits. Thus, one of skill in the art would understand that the affinity of an AAV for its target may be optimized by providing chimeric AAVs. Of note, VP1 and VP2 are present in a lower proportion than VP3 in the AAV capsid; thus, in some embodiments, provided herein is a chimeric AAV comprising amino acid substitution mutations in only VP1, only VP2, or only VP1 and VP2. In other embodiments, provided herein is a chimeric AAV comprising amino acid substitution mutations in only VP3.

AAVs comprising capsids having differences among the VP1, VP2, and/or VP3 sequences may be generated using molecular biology techniques that are known in the art. For example, the VP1, VP2, and/or VP3 may be expressed on separate plasmids or separate open reading frames (ORFs) on the same plasmid.

In other aspects, the disclosure provides a virus-like particle (VLP) having a capsid comprising only one or two of VP1, VP2, and VP3, e.g., comprising only VP1; comprising only VP2; comprising only VP3; comprising only VP1 and VP2; comprising only VP1 and VP3; or comprising only VP2 and VP3. In aspects comprising two of VP1, VP2, and VP3, one or both of the proteins may comprise the specified amino acid substitution mutation(s).

### H. Additional Mutations

In some aspects, the major capsid protein VP1, minor capsid protein VP2, and/or minor capsid protein VP3 of any of the above-described aspects comprise one or more additional amino acid substitution mutations and/or comprise one or more amino acid insertions or deletions.

In some aspects, the one or more additional amino acid substitution mutations, amino acid insertions, or amino acid deletions are substitutions, insertions, or deletions that affect binding of the AAV capsid to one or more attachment receptors. Exemplary mutations that affect (e.g., attenuate) binding to attachment receptors are provided, e.g., in Cabanes-Creus et al., Molecular Therapy: Methods and Clinical Development, 17: 1139-1154, 2020; Shen et al., Journal of Virology, 86(19): 10408-10417, 2012; and Asokan et al., Journal of Virology, 80(18): 8961-8969, 2006.

In some aspects, the modified AAV or VLP has improved transduction into liver cells (e.g., human liver cells). For example, in some aspects, one or more of the mutations provided herein is combined with a R585A, R585S, R588A, R588T, T503A, or N596D amino acid substitution mutation (numbered relative to the AAV2 major capsid protein VP1) (Cabanes-Creus et al., Molecular Therapy: Methods and Clinical Development, 17: 1139-1154, 2020). In some aspects, the modified AAV has decreased affinity for heparin.

In some aspects, the modified AAV or VLP has decreased affinity for glycans. For example, in some aspects, one or more of the mutations provided herein is combined with a W503R amino acid substitution mutation (numbered relative to the AAV9 major capsid protein VP1) (Shen et al., Journal of Virology, 86(19): 10408-10417, 2012).

In some aspects, the modified AAV or VLP has decreased affinity for integrin α5β1. For example, in some aspects, one or more of the mutations provided herein is combined with a R513A amino acid substitution mutation (numbered relative to the AAV2 major capsid protein VP1) (Asokan et al., Journal of Virology, 80(18): 8961-8969, 2006).

In some aspects, the modified AAV or VLP has decreased sialic acid binding. For example, in some aspects, one or more of the mutations provided herein is combined with a L587T amino acid substitution mutation and/or a M569V amino acid substitution mutation (see Afione et al., Journal of Virology, 89(3), 2015, which is incorporated herein by reference in its entirety).

In some aspects, the modified AAV or VLP comprises a N272A amino acid substitution mutation (e.g., as described in Bell et al., Journal of Virology, 86(13), 2012, which is incorporated herein by reference in its entirety).

### I. Target Diseases

In some aspects of any of the AAVs, VLPs, and methods provided herein, the AAV or VLP is for use in treatment of a disease or condition in a subject, e.g., a human subject. Exemplary diseases and conditions that may be treated with the AAVs and VLPs presented herein include ophthalmological, neurological, or neurodegenerative diseases or conditions and proliferative diseases or conditions (e.g., cancer). For example, in some aspects, an AAVs or VLP provided herein is used in the treatment of Alzheimer's disease (AD), amyotrophic lateral sclerosis (ALS), a disease or condition (e.g., ALS) associated with the ataxin 2 (ATXN2) gene, age-related macular degeneration (AMD) (e.g., dry AMD or wet AMD), cystic fibrosis (CF), a disease or condition (e.g., CF) associated with the cystic fibrosis transmembrane conductance regulator (CFTR) gene, neuronal ceroid lipofuscinosis 2 (CLN2) disease, frontotemporal dementia (FTD), GRN-related frontotemporal lobar degeneration (FTD-GRN), Friedreich's ataxia, generalized anxiety disorder (GAD), panic disorder (PD), Huntington's disease (HTT), Parkinson's disease (PD), a disease or condition (e.g., PD) associated with the glucocerebrosidase (GBD) gene, Rett syndrome, spinal muscular atrophy (SMA), or a disease or condition associated with a Tau gene. In other aspects, the AAV or VLP is for use in an in vitro system (e.g., is for delivery to an in vitro cell).

### III. Methods of Using Engineered AAV Capsids

### A. Methods of Improving Transduction Efficiency Using Engineered AAV Capsids

### Capsids Comprising an A472H Mutation

In one aspect, the disclosure features a method of improving transduction efficiency of an adeno-associated virus (AAV) or virus-like particle (VLP), the method comprising providing a modified AAV or VLP having a capsid comprising (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation; wherein the amino acid substitution mutations are numbered relative to the AAV9 major capsid protein VP1 (SEQ ID NO: 1). In some aspects, the method comprises contacting a target cell with the modified AAV or VLP, thereby improving transduction efficiency.

In another aspect, the disclosure features a method of improving transduction efficiency of an adeno-associated virus (AAV) or virus-like particle (VLP), the method comprising contacting a target cell with a modified AAV or VLP having a capsid comprising (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation; wherein the amino acid substitution mutations are numbered relative to the AAV9 major capsid protein VP1 (SEQ ID NO: 1), thereby improving transduction efficiency. The disclosure also provides use of such AAVs or VLPs in the manufacture of a medicament for transducing a target cell and such AAVs or VLPs for use in transducing a target cell.

Thus, in some aspects, the disclosure provides use of a modified AAV in the manufacture of a medicament for transducing a target cell, wherein the modified AAV has a capsid comprising (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation; wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV or VLP of interest.

In some aspects, the disclosure provides a modified AAV for use in transducing a target cell, wherein the modified AAV has a capsid comprising (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV or VLP of interest.

In some aspects, the AAV or VLP is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV is AAV1, AAV2, or AAV9. Further serotypes that may be used in the invention are provided, e.g., in Section II(F), above.

In another aspect, the disclosure features a method of improving transduction efficiency of an AAV or VLP, the method comprising providing a modified AAV or VLP having a capsid comprising (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation, wherein the AAV is AAV9 or a variant thereof and the amino acid substitution mutations are numbered relative to the AAV9 major capsid protein VP1 (SEQ ID NO: 1). In some aspects, the method comprises contacting a target cell with the modified AAV or VLP, thereby improving transduction efficiency.

In another aspect, the disclosure features a method of improving transduction efficiency of an AAV or VLP, the method comprising contacting a target cell with a modified AAV or VLP having a capsid comprising (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation, wherein the AAV is AAV9 or a variant thereof and the amino acid substitution mutations are numbered relative to the AAV9 major capsid protein VP1 (SEQ ID NO: 1), thereby improving transduction efficiency. The disclosure also provides use of such AAVs or VLPs in the manufacture of a medicament for transducing a target cell and such AAVs or VLPs for use in transducing a target cell.

In some aspects, (a) the major capsid protein VP1 further comprises an A273N amino acid substitution mutation; (b) the minor capsid protein VP2 further comprises an A273N amino acid substitution mutation; and/or (c) the minor capsid protein VP3 further comprises an A273N amino acid substitution mutation.

In some aspects, (a) the major capsid protein VP1 further comprises a Q387K amino acid substitution mutation; (b) the minor capsid protein VP2 further comprises a Q387K amino acid substitution mutation; and/or (c) the minor capsid protein VP3 further comprises a Q387K amino acid substitution mutation.

In some aspects, (a) the major capsid protein VP1 further comprises an E500R amino acid substitution mutation; (b) the minor capsid protein VP2 further comprises an E500R amino acid substitution mutation; and/or (c) the minor capsid protein VP3 further comprises an E500R amino acid substitution mutation.

In some aspects, (a) the major capsid protein VP1 further comprises an E500P amino acid substitution mutation; (b) the minor capsid protein VP2 further comprises an E500P amino acid substitution mutation; and/or (c) the minor capsid protein VP3 further comprises an E500P amino acid substitution mutation.

In some aspects, (a) the major capsid protein VP1 further comprises A273N, Q387K, and E500R amino acid substitution mutations; (b) the minor capsid protein VP2 further comprises A273N, Q387K, and E500R amino acid substitution mutations; and/or (c) the minor capsid protein VP3 further comprises A273N, Q387K, and E500R amino acid substitution mutations.

In another aspect, the disclosure features a method of improving transduction efficiency of an AAV, the method comprising providing a modified AAV having a capsid comprising (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations, wherein the amino acid substitution mutations are numbered relative to the AAV9 major capsid protein VP1 (SEQ ID NO: 1). In some aspects, the method comprises contacting a target cell with the modified AAV, thereby improving transduction efficiency.

In another aspect, the disclosure features a method of improving transduction efficiency of an AAV, the method comprising contacting a target cell with a modified AAV having a capsid comprising (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations, wherein the amino acid substitution mutations are numbered relative to the AAV9 major capsid protein VP1 (SEQ ID NO: 1), thereby improving transduction efficiency. The disclosure also provides use of such AAVs in the manufacture of a medicament for transducing a target cell and such AAVs or VLPs for use in transducing a target cell.

Thus, in some aspects, the disclosure provides use of a modified AAV in the manufacture of a medicament for transducing a target cell, wherein the modified AAV has a capsid comprising (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some aspects, the disclosure provides a modified AAV for use in transducing a target cell, wherein the modified AAV has a capsid comprising (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some aspects, the AAV is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV is AAV1, AAV2, or AAV9.

In another aspect, the disclosure features a method of improving transduction efficiency of an AAV, the method comprising providing a modified AAV having a capsid comprising: (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations, wherein the AAV is AAV9 or a variant thereof and the amino acid substitution mutations are numbered relative to the AAV9 major capsid protein VP1 (SEQ ID NO: 1). In some aspects, the method comprises contacting a target cell with the modified AAV, thereby improving transduction efficiency.

In another aspect, the disclosure features a method of improving transduction efficiency of an AAV, the method comprising contacting a target cell with a modified AAV having a capsid comprising: (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations, wherein the AAV is AAV9 or a variant thereof and the amino acid substitution mutations are numbered relative to the AAV9 major capsid protein VP1 (SEQ ID NO: 1), thereby improving transduction efficiency. The disclosure also provides use of such AAVs in the manufacture of a medicament for transducing a target cell and such AAVs or VLPs for use in transducing a target cell.

In another aspect, the disclosure features a method of improving transduction efficiency of an AAV, the method comprising providing a modified AAV having a capsid comprising (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations; wherein the amino acid substitution mutations are numbered relative to the AAV9 major capsid protein VP1 (SEQ ID NO: 1). In some aspects, the method comprises contacting a target cell with the modified AAV, thereby improving transduction efficiency.

In another aspect, the disclosure features a method of improving transduction efficiency of an AAV, the method comprising contacting a target cell with a modified AAV having a capsid comprising (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations; wherein the amino acid substitution mutations are numbered relative to the AAV9 major capsid protein VP1 (SEQ ID NO: 1); thereby improving transduction efficiency. The disclosure also provides use of such AAVs in the manufacture of a medicament for transducing a target cell and such AAVs for use in transducing a target cell.

Thus, in some aspects, the disclosure provides use of a modified AAV in the manufacture of a medicament for transducing a target cell, wherein the modified AAV has a capsid comprising (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations; wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some aspects, the disclosure provides a modified AAV for use in transducing a target cell, wherein the modified AAV has a capsid comprising (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some embodiments of any of the above aspects, the modified AAV or VLP may comprise an A472F, A472K, A472N, A472W, or A472Y amino acid substitution mutation instead of the A472H amino acid substitution mutation.

### Capsids Comprising an E500R Mutation

In one aspect, the disclosure features a method of improving transduction efficiency of an adeno-associated virus (AAV) or virus-like particle (VLP), the method comprising providing a modified AAV or VLP having a capsid comprising (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; wherein the amino acid substitution mutations are numbered relative to the AAV9 major capsid protein VP1 (SEQ ID NO: 1). In some aspects, the method comprises contacting a target cell with the modified AAV, thereby improving transduction efficiency.

In another aspect, the disclosure features a method of improving transduction efficiency of an adeno-associated virus (AAV) or virus-like particle (VLP), the method comprising contacting a target cell with a modified AAV or VLP having a capsid comprising (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; wherein the amino acid substitution mutations are numbered relative to the AAV9 major capsid protein VP1 (SEQ ID NO: 1), thereby improving transduction efficiency. The disclosure also provides use of such AAVs or VLPs in the manufacture of a medicament for transducing a target cell and such AAVs or VLPs for use in transducing a target cell.

In some aspects, the AAV or VLP is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV is AAV1, AAV2, or AAV9. Further serotypes that may be used in the invention are provided, e.g., in Section II(F), above.

In another aspect, the disclosure features a method of improving transduction efficiency of an AAV or VLP, the method comprising providing a modified AAV or VLP having a capsid comprising (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation, wherein the AAV is AAV9 or a variant thereof and the amino acid substitution mutations are numbered relative to the AAV9 major capsid protein VP1 (SEQ ID NO: 1). In some aspects, the method comprises contacting a target cell with the modified AAV or VLP, thereby improving transduction efficiency.

In another aspect, the disclosure features a method of improving transduction efficiency of an AAV or VLP, the method comprising contacting a target cell with a modified AAV or VLP having a capsid comprising (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation, wherein the AAV is AAV9 or a variant thereof and the amino acid substitution mutations are numbered relative to the AAV9 major capsid protein VP1 (SEQ ID NO: 1), thereby improving transduction efficiency. The disclosure also provides use of such AAVs or VLPs in the manufacture of a medicament for transducing a target cell and such AAVs or VLPs for use in transducing a target cell.

In some aspects, (a) the major capsid protein VP1 further comprises an A273N amino acid substitution mutation; (b) the minor capsid protein VP2 further comprises an A273N amino acid substitution mutation; and/or (c) the minor capsid protein VP3 further comprises an A273N amino acid substitution mutation.

In some aspects, (a) the major capsid protein VP1 further comprises a Q387K amino acid substitution mutation; (b) the minor capsid protein VP2 further comprises a Q387K amino acid substitution mutation; and/or (c) the minor capsid protein VP3 further comprises a Q387K amino acid substitution mutation.

In some aspects, (a) the major capsid protein VP1 further comprises an A472H amino acid substitution mutation; (b) the minor capsid protein VP2 further comprises an A472H amino acid substitution mutation; and/or (c) the minor capsid protein VP3 further comprises an A472H amino acid substitution mutation.

In some aspects, (a) the major capsid protein VP1 further comprises A273N and Q387K amino acid substitution mutations; (b) the minor capsid protein VP2 further comprises A273N and Q387K amino acid substitution mutations; and/or (c) the minor capsid protein VP3 further comprises A273N and Q387K amino acid substitution mutations.

In some aspects, (a) the major capsid protein VP1 further comprises A273N, Q387K, and A472H amino acid substitution mutations; (b) the minor capsid protein VP2 further comprises A273N, Q387K, and A472H amino acid substitution mutations; and/or (c) the minor capsid protein VP3 further comprises A273N, Q387K, and A472H amino acid substitution mutations.

In another aspect, the disclosure features a method of improving transduction efficiency of an AAV, the method comprising providing a modified AAV having a capsid comprising (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations, wherein the amino acid substitution mutations are numbered relative to the AAV9 major capsid protein VP1 (SEQ ID NO: 1). In some aspects, the method comprises contacting a target cell with the modified AAV, thereby improving transduction efficiency.

In another aspect, the disclosure features a method of improving transduction efficiency of an AAV, the method comprising contacting a target cell with a modified AAV having a capsid comprising (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations, wherein the amino acid substitution mutations are numbered relative to the AAV9 major capsid protein VP1 (SEQ ID NO: 1), thereby improving transduction efficiency. The disclosure also provides use of such AAVs in the manufacture of a medicament for transducing a target cell and such AAVs for use in transducing a target cell.

Thus, in some aspects, the disclosure provides use of a modified AAV in the manufacture of a medicament for transducing a target cell, wherein the modified AAV has a capsid comprising (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some aspects, the disclosure provides a modified AAV for use in transducing a target cell, wherein the modified AAV has a capsid comprising (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some aspects, (a) the major capsid protein VP1 further comprises an A472H amino acid substitution mutation; (b) the minor capsid protein VP2 further comprises an A472H amino acid substitution mutation; and/or (c) the minor capsid protein VP3 further comprises an A472H amino acid substitution mutation.

In some aspects, the AAV is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV is AAV1, AAV2, or AAV9.

In another aspect, the disclosure features a method of improving transduction efficiency of an AAV, the method comprising providing a modified AAV having a capsid comprising: (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations, wherein the AAV is AAV9 or a variant thereof and the amino acid substitution mutations are numbered relative to the AAV9 major capsid protein VP1 (SEQ ID NO: 1). In some aspects, the method comprises contacting a target cell with the modified AAV, thereby improving transduction efficiency.

In another aspect, the disclosure features a method of improving transduction efficiency of an AAV, the method comprising contacting a target cell with a modified AAV having a capsid comprising: (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations, wherein the AAV is AAV9 or a variant thereof and the amino acid substitution mutations are numbered relative to the AAV9 major capsid protein VP1 (SEQ ID NO: 1), thereby improving transduction efficiency.

### Alternative Amino Acid Substitution at Position E500

In some embodiments of any of the aspects provided herein, the modified AAV or VLP may comprise an E500P amino acid substitution mutation instead of the E500R amino acid substitution mutation.

### Capsids Comprising T592D and A593E Mutations

In another aspect, the disclosure features a method of improving transduction efficiency of an AAV or VLP, the method comprising providing a modified AAV having a capsid comprising (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations, wherein the amino acid substitution mutations are numbered relative to the AAV2 major capsid protein VP1 (SEQ ID NO: 2). In some aspects, the method comprises contacting a target cell with the modified AAV or VLP, thereby improving transduction efficiency.

In another aspect, the disclosure features a method of improving transduction efficiency of an AAV or VLP, the method comprising contacting a target cell with a modified AAV having a capsid comprising (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations, wherein the amino acid substitution mutations are numbered relative to the AAV2 major capsid protein VP1 (SEQ ID NO: 2), thereby improving transduction efficiency. The disclosure also provides use of such AAVs or VLPs in the manufacture of a medicament for transducing a target cell and such AAVs or VLPs for use in transducing a target cell.

Thus, in some aspects, the disclosure provides use of a modified AAV in the manufacture of a medicament for transducing a target cell, wherein the modified AAV has a capsid comprising (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations; wherein the amino acid substitution mutations are numbered relative to the AAV2 major capsid protein VP1 (SEQ ID NO: 2), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV or VLP of interest.

In some aspects, the disclosure provides a modified AAV for use in transducing a target cell, wherein the modified AAV has a capsid comprising (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations, wherein the amino acid substitution mutations are numbered relative to the AAV2 major capsid protein VP1 (SEQ ID NO: 2), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV or VLP of interest.

In some aspects, the AAV is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV is AAV1, AAV2, or AAV9.

In another aspect, the disclosure features a method of improving transduction efficiency of an adeno-associated virus (AAV), the method comprising providing a modified AAV having a capsid comprising (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations, wherein the AAV is AAV2 or a variant thereof and the amino acid substitution mutations are numbered relative to the AAV2 major capsid protein VP1 (SEQ ID NO: 2). In some aspects, the method comprises contacting a target cell with the modified AAV, thereby improving transduction efficiency.

In another aspect, the disclosure features a method of improving transduction efficiency of an adeno-associated virus (AAV), the method comprising contacting a target cell with a modified AAV having a capsid comprising (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations, wherein the AAV is AAV2 or a variant thereof and the amino acid substitution mutations are numbered relative to the AAV2 major capsid protein VP1 (SEQ ID NO: 2), thereby improving transduction efficiency. The disclosure also provides use of such AAVs in the manufacture of a medicament for transducing a target cell and such AAVs for use in transducing a target cell.

### Capsids Comprising I451Y, S454I, and P504I Mutations

In another aspect, the disclosure features a method of improving transduction efficiency of an adeno-associated virus (AAV) or virus-like particle (VLP), the method comprising providing a modified AAV or VLP having a capsid comprising (a) a major capsid protein VP1 comprising I451Y, S454I, and/or P504I amino acid substitution mutations; (b) a minor capsid protein VP2 comprising I451Y, S454I, and/or P504I amino acid substitution mutations; and/or (c) a minor capsid protein VP3 comprising I451Y, S454I, and/or P504I amino acid substitution mutations; wherein the amino acid substitution mutations are numbered relative to the AAV9 major capsid protein VP1 (SEQ ID NO: 1). In some aspects, the method comprises contacting a target cell with the modified AAV or VLP, thereby improving transduction efficiency.

In another aspect, the disclosure features a method of improving transduction efficiency of an adeno-associated virus (AAV) or virus-like particle (VLP), the method comprising providing a modified AAV or VLP having a capsid comprising (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations; wherein the amino acid substitution mutations are numbered relative to the AAV9 major capsid protein VP1 (SEQ ID NO: 1). In some aspects, the method comprises contacting a target cell with the modified AAV or VLP, thereby improving transduction efficiency.

In another aspect, the disclosure features a method of improving transduction efficiency of an adeno-associated virus (AAV) or virus-like particle (VLP), the method comprising contacting a target cell with a modified AAV or VLP having a capsid comprising (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations; wherein the amino acid substitution mutations are numbered relative to the AAV9 major capsid protein VP1 (SEQ ID NO: 1), thereby improving transduction efficiency. The disclosure also provides use of such AAVs or VLPs in the manufacture of a medicament for transducing a target cell and such AAVs or VLPs for use in transducing a target cell.

Thus, in some aspects, the disclosure provides use of a modified AAV in the manufacture of a medicament for transducing a target cell, wherein the modified AAV has a capsid comprising (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations; wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV or VLP of interest.

In some aspects, the disclosure provides a modified AAV for use in transducing a target cell, wherein the modified AAV has a capsid comprising (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV or VLP of interest.

In some aspects, the AAV or VLP is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV is AAV1, AAV2, or AAV9. Further serotypes that may be used in the invention are provided, e.g., in Section II(F), above.

In another aspect, the disclosure features a method of improving transduction efficiency of an AAV or VLP, the method comprising providing a modified AAV or VLP having a capsid comprising (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations, wherein the AAV is AAV9 or a variant thereof and the amino acid substitution mutations are numbered relative to the AAV9 major capsid protein VP1 (SEQ ID NO: 1). In some aspects, the method comprises contacting a target cell with the modified AAV or VLP, thereby improving transduction efficiency.

In another aspect, the disclosure features a method of improving transduction efficiency of an AAV or VLP, the method comprising contacting a target cell with a modified AAV or VLP having a capsid comprising (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations, wherein the AAV is AAV9 or a variant thereof and the amino acid substitution mutations are numbered relative to the AAV9 major capsid protein VP1 (SEQ ID NO: 1), thereby improving transduction efficiency. The disclosure also provides use of such AAVs or VLPs in the manufacture of a medicament for transducing a target cell and such AAVs or VLPs for use in transducing a target cell.

In some aspects, (a) the major capsid protein VP1 further comprises an E500P amino acid substitution mutation; (b) the minor capsid protein VP2 further comprises an E500P amino acid substitution mutation; and/or (c) the minor capsid protein VP3 further comprises an E500P amino acid substitution mutation.

In some aspects, (a) the major capsid protein VP1 further comprises an E500R amino acid substitution mutation; (b) the minor capsid protein VP2 further comprises an E500R amino acid substitution mutation; and/or (c) the minor capsid protein VP3 further comprises an E500R amino acid substitution mutation.

In another aspect, the disclosure features a method of improving transduction efficiency of an AAV, the method comprising providing a modified AAV having a capsid comprising (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations, wherein the amino acid substitution mutations are numbered relative to the AAV9 major capsid protein VP1 (SEQ ID NO: 1). In some aspects, the method comprises contacting a target cell with the modified AAV, thereby improving transduction efficiency.

In another aspect, the disclosure features a method of improving transduction efficiency of an AAV, the method comprising contacting a target cell with a modified AAV having a capsid comprising (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations, wherein the amino acid substitution mutations are numbered relative to the AAV9 major capsid protein VP1 (SEQ ID NO: 1), thereby improving transduction efficiency.

In another aspect, the disclosure features a method of improving transduction efficiency of an AAV, the method comprising contacting a target cell with a modified AAV having a capsid comprising: (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations, wherein the AAV is AAV9 or a variant thereof and the amino acid substitution mutations are numbered relative to the AAV9 major capsid protein VP1 (SEQ ID NO: 1), thereby improving transduction efficiency.

In another aspect, the disclosure features a method of improving transduction efficiency of an AAV, the method comprising providing a modified AAV having a capsid comprising (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations, wherein the amino acid substitution mutations are numbered relative to the AAV9 major capsid protein VP1 (SEQ ID NO: 1). In some aspects, the method comprises contacting a target cell with the modified AAV, thereby improving transduction efficiency.

In another aspect, the disclosure features a method of improving transduction efficiency of an AAV, the method comprising contacting a target cell with a modified AAV having a capsid comprising (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations, wherein the amino acid substitution mutations are numbered relative to the AAV9 major capsid protein VP1 (SEQ ID NO: 1), thereby improving transduction efficiency.

In another aspect, the disclosure features a method of improving transduction efficiency of an AAV, the method comprising contacting a target cell with a modified AAV having a capsid comprising: (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations, wherein the AAV is AAV9 or a variant thereof and the amino acid substitution mutations are numbered relative to the AAV9 major capsid protein VP1 (SEQ ID NO: 1), thereby improving transduction efficiency.

The disclosure also provides use of the above-described AAVs or VLPs in the manufacture of a medicament for transducing a target cell and such AAVs or VLPs for use in transducing a target cell.

Thus, in some aspects, the disclosure provides use of a modified AAV in the manufacture of a medicament for transducing a target cell, wherein the modified AAV has a capsid comprising (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV or VLP of interest.

In some aspects, the disclosure provides a modified AAV for use in transducing a target cell, wherein the modified AAV has a capsid comprising (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV or VLP of interest.

In some aspects, the disclosure provides use of a modified AAV in the manufacture of a medicament for transducing a target cell, wherein the modified AAV has a capsid comprising (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations; wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV or VLP of interest.

In some aspects, the disclosure provides a modified AAV for use in transducing a target cell, wherein the modified AAV has a capsid comprising (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV or VLP of interest.

### Alternative Amino Acid Substitutions at Position I451

In some embodiments of any of the aspects provided herein, the modified AAV or VLP may comprise an I451Y or I451M amino acid substitution mutation instead of the I451T amino acid substitution mutation.

### Alternative Amino Acid Substitution at Position S454

In some embodiments of any of the aspects provided herein, the modified AAV or VLP may comprise a S454F amino acid substitution mutation instead of the S454I amino acid substitution mutation.

### Alternative Amino Acid Substitutions at Position P504

In some embodiments of any of the aspects provided herein, the modified AAV or VLP may comprise a P504T or P504V amino acid substitution mutation instead of the P504I amino acid substitution mutation.

### Further Capsid Mutations

In another aspect, the disclosure features a method of improving transduction efficiency of an adeno-associated virus (AAV) or virus-like particle (VLP), the method comprising providing a modified AAV or VLP having a capsid comprising (a) a major capsid protein VP1; (b) a minor capsid protein VP2; and/or (c) a minor capsid protein VP3 comprising one or more of an I451T, I451Y, or I451M amino acid substitution mutation; an S454I or S454F amino acid substitution mutation; an A472F, A472H, A472K, A472N, A472W, or A472Y amino acid substitution mutation; a P504I, P504T, or P504V amino acid substitution mutation; an E500P or E500R amino acid substitution mutation; an A273N amino acid substitution mutation; a Q387K amino acid substitution mutation; an S263A or S263Y amino acid substitution mutation; and a W503H amino acid substitution mutation, wherein the amino acid substitution mutations are numbered relative to the AAV9 major capsid protein VP1 (SEQ ID NO: 1). In some aspects, the method comprises contacting a target cell with the modified AAV, thereby improving transduction efficiency.

In another aspect, the disclosure features a method of improving transduction efficiency of an adeno-associated virus (AAV) or virus-like particle (VLP), the method comprising contacting a target cell with a modified AAV or VLP having a capsid comprising (a) a major capsid protein VP1; (b) a minor capsid protein VP2; and/or (c) a minor capsid protein VP3 comprising one or more of an I451T, I451Y, or I451M amino acid substitution mutation; an S454I or S454F amino acid substitution mutation; an A472F, A472H, A472K, A472N, A472W, or A472Y amino acid substitution mutation; a P504I, P504T, or P504V amino acid substitution mutation; an E500P or E500R amino acid substitution mutation; an A273N amino acid substitution mutation; a Q387K amino acid substitution mutation; an S263A or S263Y amino acid substitution mutation; and a W503H amino acid substitution mutation; wherein the amino acid substitution mutations are numbered relative to the AAV9 major capsid protein VP1 (SEQ ID NO: 1), thereby improving transduction efficiency. The disclosure also provides use of such AAVs or VLPs in the manufacture of a medicament for transducing a target cell and such AAVs or VLPs for use in transducing a target cell.

Thus, in some aspects, the disclosure provides use of a modified AAV in the manufacture of a medicament for transducing a target cell, wherein the modified AAV has a capsid comprising (a) a major capsid protein VP1; (b) a minor capsid protein VP2; and/or (c) a minor capsid protein VP3 comprising one or more of an I451T, I451Y, or I451M amino acid substitution mutation; an S454I or S454F amino acid substitution mutation; an A472F, A472H, A472K, A472N, A472W, or A472Y amino acid substitution mutation; a P504I, P504T, or P504V amino acid substitution mutation; an E500P or E500R amino acid substitution mutation; an A273N amino acid substitution mutation; a Q387K amino acid substitution mutation; an S263A or S263Y amino acid substitution mutation; and a W503H amino acid substitution mutation; wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV or VLP of interest.

In some aspects, the disclosure provides a modified AAV for use in transducing a target cell, wherein the modified AAV has a capsid comprising (a) a major capsid protein VP1; (b) a minor capsid protein VP2; and/or (c) a minor capsid protein VP3 comprising one or more of an I451T, I451Y, or I451M amino acid substitution mutation; an S454I or S454F amino acid substitution mutation; an A472F, A472H, A472K, A472N, A472W, or A472Y amino acid substitution mutation; a P504I, P504T, or P504V amino acid substitution mutation; an E500P or E500R amino acid substitution mutation; an A273N amino acid substitution mutation; a Q387K amino acid substitution mutation; an S263A or S263Y amino acid substitution mutation; and a W503H amino acid substitution mutation, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV or VLP of interest.

In some aspects, the AAV or VLP comprises two, three, four, five, six, seven, eight, or nine of the listed amino acid substitution mutations. For example, in some aspects, the AAV or VLP has a capsid comprising (a) a major capsid protein VP1; (b) a minor capsid protein VP2; and/or (c) a minor capsid protein VP3 comprising I451T and A472H, I451Y and A472H, S454I and A472H, A472H and P504I, A472H and P504T, A472H and E500R, A273N and A472H, A472H and E500P, Q387K and A472H, A273N and Q387K, A273N and E500R, or Q387K and E500R amino acid substitution mutations. In some aspects, the AAV or VLP has a capsid comprising (a) a major capsid protein VP1; (b) a minor capsid protein VP2; and/or (c) a minor capsid protein VP3 comprising Q387K, A472H, and E500P; A273N, A472H, and E500P; I451Y, S454I, and E500P; or A273N, Q387K, and E500R amino acid substitution mutations. In some aspects, the AAV or VLP has a capsid comprising (a) a major capsid protein VP1; (b) a minor capsid protein VP2; and/or (c) a minor capsid protein VP3 comprising I451Y, S454I, E500P, and P504I; I451Y, S454I, E500P, and P504T; or A273N, Q387K, A472H, and E500P amino acid substitution mutations.

In some aspects, the AAV or VLP is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV is AAV1, AAV2, or AAV9. Further serotypes that may be used in the invention are provided, e.g., in Section II(F), above.

### Improvement of Transduction Efficiency

In some aspects, transduction efficiency of the modified AAV or VLP is improved by at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, or more than 200%, e.g., is improved by 5%-10%, 10%-20%, 20%-30%, 30%-40%, 40%-50%, 50%-60%, 60%-70%, 70%-80%, 80%-90%, 90%-100%, 100%-150%, or 150%-200% relative to the transduction efficiency of a control AAV or VLP having a capsid that (a) does not comprise any of the indicated amino acid substitution mutations or (b) comprises fewer of the indicated amino acid substitution mutations than the modified AAV or VLP of interest (e.g., an AAV or VLP of the same serotype that does not comprise any of the indicated amino acid substitution mutations or comprises fewer of the indicated amino acid substitution mutations than the modified AAV or VLP of interest).

The improvement in transduction efficiency may be in one or more target cells. Exemplary target cells include, without limitation, an endothelial cell (e.g., a brain endothelial cell), a cancer cell, a central nervous system (CNS) cell, an eye cell, a retinal cell, a muscle cell, a stem cell, a heart cell, a lung cell, a skin cell, a kidney cell, and a liver cell. In some aspects, the target cell is an in vitro cell. In other aspects, the target cell is a cell in an organism (e.g., a mammal, e.g., a human).

In some aspects, the modified AAV or VLP has an increased transduction efficiency relative to a control AAV or VLP having a capsid that (a) does not comprise any of the indicated amino acid substitution mutations or (b) comprises fewer of the indicated amino acid substitution mutations than the modified AAV or VLP of interest (e.g., an AAV or VLP of the same serotype that does not comprise any of the indicated amino acid substitution mutations or comprises fewer of the indicated amino acid substitution mutations than the modified AAV or VLP of interest).

In some aspects, the modified AAV or VLP has an increased transduction efficiency relative to an AAV or a VLP derivative thereof having a capsid that does not comprise any of the A273N, Q387K, A472H, and E500R amino acid substitution mutations. In some aspects in which the modified AAV or VLP comprises two of the four A273N, Q387K, A472H, and E500R amino acid substitution mutations (e.g., comprises A273N and Q387K; A273N and A472H; A273N and E500R; Q387K and A472H; Q387K and E500R; or A472H and E500R amino acid substitution mutations), the modified AAV or VLP has an increased transduction efficiency relative to a control AAV or VLP having a capsid that comprises only one of the indicated amino acid substitution mutations. In some aspects, the modified AAV or VLP comprises A273N and A472H amino acid substitution mutations; comprises A273N and E500R amino acid substitution mutations; comprises Q387K and A472H amino acid substitution mutations; comprises Q387K and E500R amino acid substitution mutations; or comprises A472H and E500R amino acid substitution mutations and the modified AAV or VLP has an increased transduction efficiency relative to a control AAV or VLP having a capsid that comprises only one of the indicated amino acid substitution mutations. In some aspects in which the modified AAV or VLP comprises three of the four A273N, Q387K, A472H, and E500R amino acid substitution mutations (e.g., comprises A273N, Q387K, and A472H; A273N, Q387K, and E500R; A273N, A472H, and E500R; or Q387K, A472H, and E500R amino acid substitution mutations), the modified AAV or VLP has an increased transduction efficiency relative to a control AAV or VLP having a capsid that comprises only one or only two of the indicated amino acid substitution mutations. In some aspects, the modified AAV or VLP comprises A273N, Q387K, and A472H amino acid substitution mutations; comprises A273N, Q387K, and E500R amino acid substitution mutations; comprises A273N, A472H, and E500R amino acid substitution mutations; or comprises Q387K, A472H, and E500R amino acid substitution mutations and the modified AAV or VLP has an increased transduction efficiency relative to a control AAV or VLP having a capsid that comprises only one or only two of the indicated amino acid substitution mutations. In some aspects in which the modified AAV or VLP comprises all four of the A273N, Q387K, A472H, and E500R amino acid substitution mutations, the modified AAV or VLP has an increased transduction efficiency relative to a control AAV or VLP having a capsid that comprises only one, only two, or only three of the indicated amino acid substitution mutations. In some aspects, the modified AAV or VLP is AAV9 or a variant thereof.

In some aspects, the modified AAV or VLP is AAV2 or a variant thereof and has an increased transduction efficiency relative to an AAV2 or a VLP derivative thereof having a capsid that (a) does not comprise either of the T592D and A593E amino acid substitution mutations or (b) comprises only one of the T592D and A593E amino acid substitution mutations.

For example, in some aspects, transduction efficiency is increased by at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, or more than 200%, e.g., is improved by 5%-10%, 10%-20%, 20%-30%, 30%-40%, 40%-50%, 50%-60%, 60%-70%, 70%-80%, 80%-90%, 90%-100%, 100%-150%, or 150%-200% relative to the affinity of a control AAV or VLP.

In some aspects, the modified AAV or VLP has an increased rate of delivery to the nucleus of a target cell relative to a control AAV or VLP having a capsid that (a) does not comprise any of the indicated amino acid substitution mutations or (b) comprises fewer of the indicated amino acid substitution mutations than the modified AAV or VLP of interest (e.g., an AAV or VLP of the same serotype that does not comprise any of the indicated amino acid substitution mutations or comprises fewer of the indicated amino acid substitution mutations than the modified AAV or VLP of interest).

In some aspects, the modified AAV or VLP has an increased rate of delivery to the nucleus of the target cell relative to an AAV or a VLP derivative thereof having a capsid that does not comprise any of the A273N, Q387K, A472H, and E500R substitution mutations. In some aspects in which the modified AAV or VLP comprises two of the four A273N, Q387K, A472H, and E500R amino acid substitution mutations (e.g., comprises A273N and Q387K; A273N and A472H; A273N and E500R; Q387K and A472H; Q387K and E500R; or A472H and E500R amino acid substitution mutations), the modified AAV or VLP has an increased rate of delivery to the nucleus relative to a control AAV or VLP having a capsid that comprises only one of the indicated amino acid substitution mutations. In some aspects, the modified AAV or VLP comprises A273N and A472H amino acid substitution mutations; comprises A273N and E500R amino acid substitution mutations; comprises Q387K and A472H amino acid substitution mutations; comprises Q387K and E500R amino acid substitution mutations; or comprises A472H and E500R amino acid substitution mutations and the modified AAV or VLP has an increased rate of delivery to the nucleus relative to a control AAV or VLP having a capsid that comprises only one of the indicated amino acid substitution mutations. In some aspects in which the modified AAV or VLP comprises three of the four A273N, Q387K, A472H, and E500R amino acid substitution mutations (e.g., comprises A273N, Q387K, and A472H; A273N, Q387K, and E500R; A273N, A472H, and E500R; or Q387K, A472H, and E500R amino acid substitution mutations), the modified AAV or VLP has an increased rate of delivery to the nucleus relative to a control AAV or VLP having a capsid that comprises only one or only two of the indicated amino acid substitution mutations. In some aspects, the modified AAV or VLP comprises A273N, Q387K, and A472H amino acid substitution mutations; comprises A273N, Q387K, and E500R amino acid substitution mutations; comprises A273N, A472H, and E500R amino acid substitution mutations; or comprises Q387K, A472H, and E500R amino acid substitution mutations and the modified AAV or VLP has an increased rate of delivery to the nucleus relative to a control AAV or VLP having a capsid that comprises only one or only two of the indicated amino acid substitution mutations. In some aspects in which the modified AAV or VLP comprises all four of the A273N, Q387K, A472H, and E500R amino acid substitution mutations, the modified AAV or VLP has an increased rate of delivery to the nucleus relative to a control AAV or VLP having a capsid that comprises only one, only two, or only three of the indicated amino acid substitution mutations. In some aspects, the modified AAV or VLP is AAV9 or a variant thereof.

In some aspects, the modified AAV or VLP is AAV2 or a variant thereof and has an increased rate of delivery to the nucleus of the target cell relative to an AAV2 or a VLP derivative thereof having a capsid that (a) does not comprise either of the T592D and A593E amino acid substitution mutations or (b) comprises only one of the T592D and A593E amino acid substitution mutations.

For example, in some aspects, the rate of delivery to the nucleus is increased by at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, or more than 200%, e.g., is improved by 5%-10%, 10%-20%, 20%-30%, 30%-40%, 40%-50%, 50%-60%, 60%-70%, 70%-80%, 80%-90%, 90%-100%, 100%-150%, or 150%-200% relative to the rate of delivery of a control AAV or VLP.

In some aspects, the disclosure provides a method of increasing the rate of delivery of an AAV to the nucleus of a target cell, the method comprising providing a modified AAV having a capsid comprising:
(i) (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation;
(ii) (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; or
(iii) (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations;
   wherein the amino acid substitution mutations are numbered relative to the AAV9 major capsid protein VP1 (SEQ ID NO: 1). In some aspects, the method comprises contacting a target cell with the modified AAV, thereby increasing the rate of delivery to the nucleus.

In some aspects, the disclosure provides a method of increasing the rate of delivery of an AAV to the nucleus of a target cell, the method comprising providing a modified AAV having a capsid comprising:
(i) (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; or
(ii) (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations;
   wherein the AAV is AAV9 or a variant thereof and the amino acid substitution mutations are numbered relative to the AAV9 major capsid protein VP1 (SEQ ID NO: 1). In some aspects, the method comprises contacting a target cell with the modified AAV, thereby increasing the rate of delivery to the nucleus.

In some aspects, the disclosure features a method of increasing the rate of delivery of an AAV to the nucleus of a target cell, the method comprising providing a modified AAV having a capsid comprising: (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations, wherein the AAV is AAV2 or a variant thereof and the amino acid substitution mutations are numbered relative to the AAV2 major capsid protein VP1 (SEQ ID NO: 2). In some aspects, the method comprises contacting a target cell with the modified AAV, thereby increasing the rate of delivery to the nucleus.

In some aspects, the disclosure features a method of increasing the rate of delivery of an AAV to the nucleus of a target cell, the method comprising providing a modified AAV having a capsid comprising:
(i) (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations;
(ii) (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; or
(iii) (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations;
   wherein the AAV is AAV9 or a variant thereof and the amino acid substitution mutations are numbered relative to the AAV9 major capsid protein VP1 (SEQ ID NO: 1). In some aspects, the method comprises contacting a target cell with the modified AAV, thereby increasing the rate of delivery to the nucleus.

In some aspects, the modified AAV transduces a target cell at a lower dose than a control AAV or VLP having a capsid that (a) does not comprise any of the indicated amino acid substitution mutations or (b) comprises fewer of the indicated amino acid substitution mutations than the modified AAV or VLP of interest (e.g., an AAV or VLP of the same serotype that does not comprise any of the indicated amino acid substitution mutations or comprises fewer of the indicated amino acid substitution mutations than the modified AAV or VLP of interest), e.g., transduces the target cell at a rate sufficient to achieve a desired effect, e.g., a therapeutic effect, at a lower dose.

In some aspects, the modified AAV or VLP transduces a target cell at a lower dose than an AAV or a VLP derivative thereof having a capsid that does not comprise any of the A273N, Q387K, A472H, and E500R substitution mutations. In some aspects in which the modified AAV or VLP comprises two of the four A273N, Q387K, A472H, and E500R amino acid substitution mutations (e.g., comprises A273N and Q387K; A273N and A472H; A273N and E500R; Q387K and A472H; Q387K and E500R; or A472H and E500R amino acid substitution mutations), the modified AAV or VLP transduces a target cell at a lower dose than a control AAV or VLP having a capsid that comprises only one of the indicated amino acid substitution mutations. In some aspects, the modified AAV or VLP comprises A273N and A472H amino acid substitution mutations; comprises A273N and E500R amino acid substitution mutations; comprises Q387K and A472H amino acid substitution mutations; comprises Q387K and E500R amino acid substitution mutations; or comprises A472H and E500R amino acid substitution mutations and the modified AAV or VLP transduces a target cell at a lower dose than a control AAV or VLP having a capsid that comprises only one of the indicated amino acid substitution mutations. In some aspects in which the modified AAV or VLP comprises three of the four A273N, Q387K, A472H, and E500R amino acid substitution mutations (e.g., comprises A273N, Q387K, and A472H; A273N, Q387K, and E500R; A273N, A472H, and E500R; or Q387K, A472H, and E500R amino acid substitution mutations), the modified AAV or VLP transduces a target cell at a lower dose than a control AAV or VLP having a capsid that comprises only one or only two of the indicated amino acid substitution mutations. In some aspects, the modified AAV or VLP comprises A273N, Q387K, and A472H amino acid substitution mutations; comprises A273N, Q387K, and E500R amino acid substitution mutations; comprises A273N, A472H, and E500R amino acid substitution mutations; or comprises Q387K, A472H, and E500R amino acid substitution mutations and the modified AAV or VLP transduces a target cell at a lower dose than a control AAV or VLP having a capsid that comprises only one or only two of the indicated amino acid substitution mutations. In some aspects in which the modified AAV or VLP comprises all four of the A273N, Q387K, A472H, and E500R amino acid substitution mutations, the modified AAV or VLP transduces a target cell at a lower dose than a control AAV or VLP having a capsid that comprises only one, only two, or only three of the indicated amino acid substitution mutations. In some aspects, the modified AAV or VLP is AAV9 or a variant thereof.

In some aspects, the modified AAV or VLP is AAV2 or a variant thereof and transduces a target cell at a lower dose than an AAV2 or a VLP derivative thereof having a capsid that (a) does not comprise either of the T592D and A593E amino acid substitution mutations or (b) comprises only one of the T592D and A593E amino acid substitution mutations.

For example, in some aspects, the modified AAV or VLP transduces the target cell at a dose that is at least 1.1-fold lower, 1.2-fold lower, 1.3-fold lower, 1.4-fold lower, 1.5-fold lower, 1.6-fold lower, 1.7-fold lower, 1.8-fold lower, 1.9-fold lower, 2-fold lower, 2.5-fold lower, 3-fold lower, 3.5-fold lower, 4-fold lower, 4.5-fold lower, 5-fold lower, 5.5-fold lower, 6-fold lower, 6.5-fold lower, 7-fold lower, 7.5-fold lower, 8-fold lower, 8.5-fold lower, 9-fold lower, 9.5-fold lower, 10-fold lower, or more than 10-fold lower, e.g., 1.1-1.5-fold lower, 1.5-2-fold lower, 2-3-fold lower, 3-4-fold lower, 4-5-fold lower, 5-6-fold lower, 6-7-fold lower, 7-8-fold lower, 8-9-fold lower, or 9-10-fold lower than the dose of a control AAV or VLP. Accordingly, in some aspects, the modified AAV or VLP has a lower effective dose than a control AAV or VLP that (a) does not comprise any of the indicated amino acid substitution mutations or (b) comprises fewer of the indicated amino acid substitution mutations than the modified AAV or VLP of interest.

In some aspects, the disclosure features a method of transduction of a target cell by an AAV at a lower dose of the AAV, the method comprising providing a modified AAV having a capsid comprising:
(i) (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation;
(ii) (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations; or
(iii) (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations; wherein the amino acid substitution mutations are numbered relative to the AAV9 major capsid protein VP1 (SEQ ID NO: 1). In some aspects, the method comprises contacting a target cell with the modified AAV, thereby transducing the target cell at a lower AAV dose.

In some aspects, the disclosure features a method of transduction of a target cell by an AAV at a lower dose of the AAV, the method comprising providing a modified AAV having a capsid comprising:
(i) (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; or
(ii) (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations wherein the AAV is AAV9 or a variant thereof and the amino acid substitution mutations are numbered relative to the AAV9 major capsid protein VP1 (SEQ ID NO: 1). In some aspects, the method comprises contacting a target cell with the modified AAV, thereby transducing the target cell at a lower AAV dose.

In some aspects, the disclosure features a method of transduction of a target cell by an AAV at a lower dose of the AAV, the method comprising providing a modified AAV having a capsid comprising (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations; wherein the AAV is AAV2 or a variant thereof and the amino acid substitution mutations are numbered relative to the AAV2 major capsid protein VP1 (SEQ ID NO: 2). In some aspects, the method comprises contacting a target cell with the modified AAV, thereby transducing the target cell at a lower AAV dose.

In some aspects, the disclosure features a method of transduction of a target cell by an AAV at a lower dose of the AAV, the method comprising providing a modified AAV having a capsid comprising:
(i) (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations;
(ii) (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; or
(iii) (a) a major capsid protein VP1 comprising I451Y, S454I, E500R, and P504I amino acid substitution mutations; (b) a minor capsid protein VP2 comprising I451Y, S454I, E500R, and P504I amino acid substitution mutations; and/or (c) a minor capsid protein VP3 comprising I451Y, S454I, E500R, and P504I amino acid substitution mutations; wherein the AAV is AAV9 or a variant thereof and the amino acid substitution mutations are numbered relative to the AAV9 major capsid protein VP1 (SEQ ID NO: 1). In some aspects, the method comprises contacting a target cell with the modified AAV, thereby transducing the target cell at a lower AAV dose.

### B. Methods of Delivering a Cargo to a Cell

### Capsids Comprising an A472H Mutation

In another aspect, the disclosure features a method of delivering a cargo to a cell, the method comprising contacting a target cell with a modified AAV or virus-like particle (VLP) having a capsid comprising (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation; wherein the cargo is encapsulated by the AAV or VLP, wherein the amino acid substitution mutations are numbered relative to the AAV9 major capsid protein VP1 (SEQ ID NO: 1), and wherein the AAV or VLP has an increased rate of transduction into a target cell relative to an AAV or VLP having a capsid (a) not comprising any of the indicated amino acid substitution mutations or (b) comprising fewer of the indicated amino acid substitution mutations than the modified AAV or VLP of interest. The disclosure also provides use of such AAVs or VLPs in the manufacture of a medicament for delivering a cargo to a target cell and such AAVs or VLPs for use in delivering a cargo to a target cell.

Thus, in some aspects, the disclosure provides use of a modified AAV in the manufacture of a medicament for delivering a cargo to a target cell, wherein the modified AAV has a capsid comprising (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation, wherein the cargo is encapsulated by the AAV, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some aspects, the disclosure provides a modified AAV for use in delivering a cargo to a target cell, wherein the modified AAV has a capsid comprising (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation, wherein the cargo is encapsulated by the AAV, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some aspects, the AAV or VLP is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV is AAV1, AAV2, or AAV9. Further serotypes that may be used in the invention are provided, e.g., in Section II(F), above.

In another aspect, the disclosure features a method of delivering a cargo to a cell, the method comprising contacting a target cell with a modified AAV or VLP having a capsid comprising: (a) a major capsid protein VP1 comprising an A472H amino acid substitution mutation; (b) a minor capsid protein VP2 comprising an A472H amino acid substitution mutation; and/or (c) a minor capsid protein VP3 comprising an A472H amino acid substitution mutation, wherein the cargo is encapsulated by the AAV or VLP; wherein the AAV or VLP is AAV9 or a variant thereof and the amino acid substitution mutations are numbered relative to the AAV9 major capsid protein VP1 (SEQ ID NO: 1), and wherein the AAV or VLP has an increased rate of transduction into a target cell relative to an AAV9 or VLP derivative thereof having a capsid (a) not comprising any of the indicated amino acid substitution mutations or (b) comprising fewer of the indicated amino acid substitution mutations than the modified AAV or VLP of interest. The disclosure also provides use of such AAVs or VLPs in the manufacture of a medicament for delivering a cargo to a target cell and such AAVs or VLPs for use in delivering a cargo to a target cell.

In some aspects, (a) the major capsid protein VP1 further comprises an A273N amino acid substitution mutation; (b) the minor capsid protein VP2 further comprises an A273N amino acid substitution mutation; and/or (c) the minor capsid protein VP3 further comprises an A273N amino acid substitution mutation.

In some aspects, (a) the major capsid protein VP1 further comprises a Q387K amino acid substitution mutation; (b) the minor capsid protein VP2 further comprises a Q387K amino acid substitution mutation; and/or (c) the minor capsid protein VP3 further comprises a Q387K amino acid substitution mutation.

In some aspects, (a) the major capsid protein VP1 further comprises an E500R amino acid substitution mutation; (b) the minor capsid protein VP2 further comprises an E500R amino acid substitution mutation; and/or (c) the minor capsid protein VP3 further comprises an E500R amino acid substitution mutation.

In some aspects, (a) the major capsid protein VP1 further comprises an E500P amino acid substitution mutation; (b) the minor capsid protein VP2 further comprises an E500P amino acid substitution mutation; and/or (c) the minor capsid protein VP3 further comprises an E500P amino acid substitution mutation.

In some aspects, (a) the major capsid protein VP1 further comprises A273N and Q387K amino acid substitution mutations; (b) the minor capsid protein VP2 further comprises A273N and Q387K amino acid substitution mutations; and/or (c) the minor capsid protein VP3 further comprises A273N and Q387K amino acid substitution mutations.

In some aspects, (a) the major capsid protein VP1 further comprises A273N, Q387K, and E500R amino acid substitution mutations; (b) the minor capsid protein VP2 further comprises A273N, Q387K, and E500R amino acid substitution mutations; and/or (c) the minor capsid protein VP3 further comprises A273N, Q387K, and E500R amino acid substitution mutations.

In some aspects, the disclosure features a method of delivering a cargo to a cell, the method comprising contacting a target cell with a modified AAV or VLP having a capsid comprising (a) a major capsid protein VP1 comprising A273N, Q387K, and A472H amino acid substitution mutations; (b) a minor capsid protein VP2 comprising A273N, Q387K, and A472H amino acid substitution mutations; and/or (c) a minor capsid protein VP3 comprising A273N, Q387K, and A472H amino acid substitution mutations; wherein the cargo is encapsulated by the AAV or VLP, wherein the amino acid substitution mutations are numbered relative to the AAV9 major capsid protein VP1 (SEQ ID NO: 1), and wherein the AAV or VLP has an increased rate of transduction into a target cell relative to an AAV or VLP having a capsid not comprising any of the indicated amino acid substitution mutations. In some aspects in which the modified AAV or VLP comprises two of the three A273N, Q387K, and A472H amino acid substitution mutations (e.g., comprises A273N and Q387K; A273N and A472H; or Q387K and A472H amino acid substitution mutations), the modified AAV or VLP has an increased rate of transduction into a target cell relative to a control AAV or VLP having a capsid that comprises only one of the indicated amino acid substitution mutations. In some aspects, the modified AAV or VLP comprises A273N and A472H amino acid substitution mutations or comprises Q387K and A472H amino acid substitution mutations and the modified AAV or VLP has an increased rate of transduction into a target cell relative to a control AAV or VLP having a capsid that comprises only one of the indicated amino acid substitution mutations. In some aspects in which the modified AAV or VLP comprises all three of the A273N, Q387K, and A472H amino acid substitution mutations, the modified AAV or VLP has an increased rate of transduction into a target cell relative to a control AAV or VLP having a capsid that comprises only one or only two of the indicated amino acid substitution mutations.

In some aspects, (a) the major capsid protein VP1 further comprises an E500R amino acid substitution mutation; (b) the minor capsid protein VP2 further comprises an E500R amino acid substitution mutation; and/or (c) the minor capsid protein VP3 further comprises an E500R amino acid substitution mutation.

The disclosure also provides use of such AAVs or VLPs in the manufacture of a medicament for delivering a cargo to a target cell and such AAVs or VLPs for use in delivering a cargo to a target cell.

Thus, in some aspects, the disclosure provides use of a modified AAV in the manufacture of a medicament for delivering a cargo to a target cell, wherein the modified AAV has a capsid comprising (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations, wherein the cargo is encapsulated by the AAV, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some aspects, the disclosure provides a modified AAV for use in delivering a cargo to a target cell, wherein the modified AAV has a capsid comprising (a) a major capsid protein VP1 comprising A273N, Q387K, A472H, and E500R amino acid substitution mutations; (b) a minor capsid protein VP2 comprising A273N, Q387K, A472H, and E500R amino acid substitution mutations; and/or (c) a minor capsid protein VP3 comprising A273N, Q387K, A472H, and E500R amino acid substitution mutations, wherein the cargo is encapsulated by the AAV, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In another aspect, the disclosure features a method of delivering a cargo to a cell, the method comprising contacting a target cell with a modified AAV having a capsid comprising (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations; wherein the cargo is encapsulated by the AAV; wherein the amino acid substitution mutations are numbered relative to the AAV9 major capsid protein VP1 (SEQ ID NO: 1); and wherein the AAV has an increased rate of transduction into a target cell relative to an AAV9 having a capsid not comprising any of the indicated amino acid substitution mutations. The disclosure also provides use of such AAVs or VLPs in the manufacture of a medicament for delivering a cargo to a target cell and such AAVs or VLPs for use in delivering a cargo to a target cell.

Thus, in some aspects, the disclosure provides use of a modified AAV in the manufacture of a medicament for delivering a cargo to a target cell, wherein the modified AAV has a capsid comprising (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations, wherein the cargo is encapsulated by the AAV, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some aspects, the disclosure provides a modified AAV for use in delivering a cargo to a target cell, wherein the modified AAV has a capsid comprising (a) a major capsid protein VP1 comprising A472H and E500P amino acid substitution mutations; (b) a minor capsid protein VP2 comprising A472H and E500P amino acid substitution mutations; and/or (c) a minor capsid protein VP3 comprising A472H and E500P amino acid substitution mutations, wherein the cargo is encapsulated by the AAV, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some aspects, the AAV or VLP is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV is AAV1, AAV2, or AAV9.

In some aspects, the cargo of the AAV or VLP is a nucleic acid, e.g., a single-stranded RNA (ssRNA). In some aspects, the cargo is a therapeutic nucleic acid. In some aspects, the cargo is a nucleic acid that is useful as a gene therapy. In some aspects, the cargo of the AAV or VLP is an antisense oligonucleotide or an siRNA. In some aspects, the inverted terminal repeat (ITR) sequences of the AAV genome are retained for packaging, and the viral genes are replaced with a sequence comprising or encoding the cargo.

In some aspects, the major capsid protein VP1, minor capsid protein VP2, and/or minor capsid protein VP3 comprise one or more further amino acid substitution mutations and/or comprise one or more amino acid insertions or deletions, e.g., comprise any of the features described in Section II(H), above.

The cell may be, e.g., an endothelial cell (e.g., a brain endothelial cell), a cancer cell, a central nervous system (CNS) cell, an eye cell, a retinal cell, a muscle cell, a stem cell, a heart cell, a lung cell, a skin cell, a kidney cell, and a liver cell. In some aspects, the target cell is an in vitro cell. In other aspects, the target cell is a cell in an organism (e.g., a mammal, e.g., a human).

In some embodiments of any of the above aspects, the modified AAV or VLP may comprise an A472F, A472K, A472N, A472W, or A472Y amino acid substitution mutation instead of the A472H amino acid substitution mutation.

### Capsids Comprising an E500R Mutation

In another aspect, the disclosure features a method of delivering a cargo to a cell, the method comprising contacting a target cell with a modified AAV or virus-like particle (VLP) having a capsid comprising (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation; wherein the cargo is encapsulated by the AAV or VLP, wherein the amino acid substitution mutations are numbered relative to the AAV9 major capsid protein VP1 (SEQ ID NO: 1), and wherein the AAV or VLP has an increased rate of transduction into a target cell relative to an AAV or VLP having a capsid (a) not comprising any of the indicated amino acid substitution mutations or (b) comprising fewer of the indicated amino acid substitution mutations than the modified AAV or VLP of interest. The disclosure also provides use of such AAVs or VLPs in the manufacture of a medicament for delivering a cargo to a target cell and such AAVs or VLPs for use in delivering a cargo to a target cell.

Thus, in some aspects, the disclosure provides use of a modified AAV in the manufacture of a medicament for delivering a cargo to a target cell, wherein the modified AAV has a capsid comprising (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation, wherein the cargo is encapsulated by the AAV, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV or VLP of interest.

In some aspects, the disclosure provides a modified AAV for use in delivering a cargo to a target cell, wherein the modified AAV has a capsid comprising (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation, wherein the cargo is encapsulated by the AAV, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV or VLP of interest.

In some aspects, the AAV or VLP is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV is AAV1, AAV2, or AAV9. Further serotypes that may be used in the invention are provided, e.g., in Section II(F), above.

In another aspect, the disclosure features a method of delivering a cargo to a cell, the method comprising contacting a target cell with a modified AAV or VLP having a capsid comprising: (a) a major capsid protein VP1 comprising an E500R amino acid substitution mutation; (b) a minor capsid protein VP2 comprising an E500R amino acid substitution mutation; and/or (c) a minor capsid protein VP3 comprising an E500R amino acid substitution mutation, wherein the cargo is encapsulated by the AAV or VLP; wherein the AAV or VLP is AAV9 or a variant thereof and the amino acid substitution mutations are numbered relative to the AAV9 major capsid protein VP1 (SEQ ID NO: 1), and wherein the AAV or VLP has an increased rate of transduction into a target cell relative to an AAV9 or VLP derivative thereof having a capsid (a) not comprising any of the indicated amino acid substitution mutations or (b) comprising fewer of the indicated amino acid substitution mutations than the modified AAV or VLP of interest. The disclosure also provides use of such AAVs or VLPs in the manufacture of a medicament for delivering a cargo to a target cell and such AAVs or VLPs for use in delivering a cargo to a target cell.

In some aspects, (a) the major capsid protein VP1 further comprises an A273N amino acid substitution mutation; (b) the minor capsid protein VP2 further comprises an A273N amino acid substitution mutation; and/or (c) the minor capsid protein VP3 further comprises an A273N amino acid substitution mutation.

In some aspects, (a) the major capsid protein VP1 further comprises a Q387K amino acid substitution mutation; (b) the minor capsid protein VP2 further comprises a Q387K amino acid substitution mutation; and/or (c) the minor capsid protein VP3 further comprises a Q387K amino acid substitution mutation.

In some aspects, (a) the major capsid protein VP1 further comprises an A472H amino acid substitution mutation; (b) the minor capsid protein VP2 further comprises an A472H amino acid substitution mutation; and/or (c) the minor capsid protein VP3 further comprises an A472H amino acid substitution mutation.

In some aspects, (a) the major capsid protein VP1 further comprises A273N and Q387K amino acid substitution mutations; (b) the minor capsid protein VP2 further comprises A273N and Q387K amino acid substitution mutations; and/or (c) the minor capsid protein VP3 further comprises A273N and Q387K amino acid substitution mutations.

In some aspects, (a) the major capsid protein VP1 further comprises A273N, Q387K, and A472H amino acid substitution mutations; (b) the minor capsid protein VP2 further comprises A273N, Q387K, and A472H amino acid substitution mutations; and/or (c) the minor capsid protein VP3 further comprises A273N, Q387K, and A472H amino acid substitution mutations.

In some aspects, the disclosure features a method of delivering a cargo to a cell, the method comprising contacting a target cell with a modified AAV or VLP having a capsid comprising (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations; wherein the cargo is encapsulated by the AAV or VLP, wherein the amino acid substitution mutations are numbered relative to the AAV9 major capsid protein VP1 (SEQ ID NO: 1), and wherein the AAV or VLP has an increased rate of transduction into a target cell relative to an AAV or VLP having a capsid not comprising any of the indicated amino acid substitution mutations. In some aspects in which the modified AAV or VLP comprises two of the three A273N, Q387K, and E500R amino acid substitution mutations (e.g., comprises A273N and Q387K; A273N and E500R; or Q387K and E500R amino acid substitution mutations), the modified AAV or VLP has an increased rate of transduction into a target cell relative to a control AAV or VLP having a capsid that comprises only one of the indicated amino acid substitution mutations. In some aspects, the modified AAV or VLP comprises A273N and E500R amino acid substitution mutations or comprises Q387K and E500R amino acid substitution mutations and the modified AAV or VLP has an increased rate of transduction into a target cell relative to a control AAV or VLP having a capsid that comprises only one of the indicated amino acid substitution mutations. In some aspects in which the modified AAV or VLP comprises all three of the A273N, Q387K, and E500R amino acid substitution mutations, the modified AAV or VLP has an increased rate of transduction into a target cell relative to a control AAV or VLP having a capsid that comprises only one or only two of the indicated amino acid substitution mutations. The disclosure also provides use of such AAVs or VLPs in the manufacture of a medicament for delivering a cargo to a target cell and such AAVs or VLPs for use in delivering a cargo to a target cell.

Thus, in some aspects, the disclosure provides use of a modified AAV in the manufacture of a medicament for delivering a cargo to a target cell, wherein the modified AAV has a capsid comprising (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations, wherein the cargo is encapsulated by the AAV, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some aspects, the disclosure provides a modified AAV for use in delivering a cargo to a target cell, wherein the modified AAV has a capsid comprising (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations, wherein the cargo is encapsulated by the AAV, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some aspects, (a) the major capsid protein VP1 further comprises an A472H amino acid substitution mutation; (b) the minor capsid protein VP2 further comprises an A472H amino acid substitution mutation; and/or (c) the minor capsid protein VP3 further comprises an A472H amino acid substitution mutation.

In some aspects, the AAV or VLP is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV is AAV1, AAV2, or AAV9.

In some aspects, the disclosure features a method of delivering a cargo to a cell, the method comprising contacting a target cell with a modified AAV or VLP having a capsid comprising (a) a major capsid protein VP1 comprising A273N, Q387K, and E500R amino acid substitution mutations; (b) a minor capsid protein VP2 comprising A273N, Q387K, and E500R amino acid substitution mutations; and/or (c) a minor capsid protein VP3 comprising A273N, Q387K, and E500R amino acid substitution mutations; wherein the cargo is encapsulated by the AAV or VLP, wherein the AAV or VLP is AAV9 or a variant thereof and the amino acid substitution mutations are numbered relative to the AAV9 major capsid protein VP1 (SEQ ID NO: 1), and wherein the AAV or VLP has an increased rate of transduction into a target cell relative to an AAV9 or VLP derivative thereof having a capsid not comprising any of the indicated amino acid substitution mutations. In some aspects in which the modified AAV or VLP comprises two of the three A273N, Q387K, and E500R amino acid substitution mutations (e.g., comprises A273N and Q387K; A273N and E500R; or Q387K and E500R amino acid substitution mutations), the modified AAV or VLP has an increased rate of transduction into a target cell relative to a control AAV or VLP having a capsid that comprises only one of the indicated amino acid substitution mutations. In some aspects, the modified AAV or VLP comprises A273N and E500R amino acid substitution mutations or comprises Q387K and E500R amino acid substitution mutations and the modified AAV or VLP has an increased rate of transduction into a target cell relative to a control AAV or VLP having a capsid that comprises only one of the indicated amino acid substitution mutations. In some aspects in which the modified AAV or VLP comprises all three of the A273N, Q387K, and E500R amino acid substitution mutations, the modified AAV or VLP has an increased rate of transduction into a target cell relative to a control AAV or VLP having a capsid that comprises only one or only two of the indicated amino acid substitution mutations. The disclosure also provides use of such AAVs or VLPs in the manufacture of a medicament for delivering a cargo to a target cell and such AAVs or VLPs for use in delivering a cargo to a target cell.

In some aspects, (a) the major capsid protein VP1 further comprises an A472H amino acid substitution mutation; (b) the minor capsid protein VP2 further comprises an A472H amino acid substitution mutation; and/or (c) the minor capsid protein VP3 further comprises an A472H amino acid substitution mutation.

In some aspects, the cargo of the AAV or VLP is a nucleic acid, e.g., a single-stranded RNA (ssRNA). In some aspects, the cargo is a therapeutic nucleic acid. In some aspects, the cargo is a nucleic acid that is useful as a gene therapy. In some aspects, the cargo of the AAV or VLP is an antisense oligonucleotide or an siRNA. In some aspects, the inverted terminal repeat (ITR) sequences of the AAV genome are retained for packaging, and the viral genes are replaced with a sequence comprising or encoding the cargo.

In some aspects, the major capsid protein VP1, minor capsid protein VP2, and/or minor capsid protein VP3 comprise one or more further amino acid substitution mutations and/or comprise one or more amino acid insertions or deletions, e.g., comprise any of the features described in Section II(H), above.

The cell may be, e.g., an endothelial cell (e.g., a brain endothelial cell), a cancer cell, a central nervous system (CNS) cell, an eye cell, a retinal cell, a muscle cell, a stem cell, a heart cell, a lung cell, a skin cell, a kidney cell, and a liver cell. In some aspects, the target cell is an in vitro cell. In other aspects, the target cell is a cell in an organism (e.g., a mammal, e.g., a human).

In some embodiments of any of the above aspects, the modified AAV or VLP may comprise an A472F, A472K, A472N, A472W, or A472Y amino acid substitution mutation instead of the A472H amino acid substitution mutation.

### Capsids Comprising T592D and A593E Mutations

In another aspect, the disclosure features a method of delivering a cargo to a cell, the method comprising contacting a target cell with a modified AAV or VLP having a capsid comprising (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations, wherein the cargo is encapsulated by the AAV; wherein the amino acid substitution mutations are numbered relative to the AAV2 major capsid protein VP1 (SEQ ID NO: 2), and wherein the AAV has an increased rate of transduction into a target cell relative to an AAV having a capsid (a) not comprising either of the indicated amino acid substitution mutations or (b) comprising only one of the T592D and A593E amino acid substitution mutations.

The disclosure also provides use of such AAVs or VLPs in the manufacture of a medicament for delivering a cargo to a target cell and such AAVs or VLPs for use in delivering a cargo to a target cell.

Thus, in some aspects, the disclosure provides use of a modified AAV in the manufacture of a medicament for delivering a cargo to a target cell, wherein the modified AAV has a capsid comprising (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations, wherein the cargo is encapsulated by the AAV, wherein the amino acid substitution mutations are numbered relative to the AAV2 major capsid protein VP1 (SEQ ID NO: 2), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some aspects, the disclosure provides a modified AAV for use in delivering a cargo to a target cell, wherein the modified AAV has a capsid comprising (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations, wherein the cargo is encapsulated by the AAV, wherein the amino acid substitution mutations are numbered relative to the AAV2 major capsid protein VP1 (SEQ ID NO: 2), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some aspects, the AAV or VLP is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV is AAV1, AAV2, or AAV9.

In another aspect, the disclosure features a method of delivering a cargo to a cell, the method comprising contacting a target cell with a modified AAV or VLP having a capsid comprising (a) a major capsid protein VP1 comprising T592D and A593E amino acid substitution mutations; (b) a minor capsid protein VP2 comprising T592D and A593E amino acid substitution mutations; and/or (c) a minor capsid protein VP3 comprising T592D and A593E amino acid substitution mutations, wherein the cargo is encapsulated by the AAV or VLP; wherein the AAV or VLP is AAV2 or a variant thereof and the amino acid substitution mutations are numbered relative to the AAV2 major capsid protein VP1 (SEQ ID NO: 2), and wherein the AAV or VLP has an increased rate of transduction into a target cell relative to an AAV2 or a VLP derivative thereof having a capsid (a) not comprising either of the indicated amino acid substitution mutations or (b) comprising only one of the T592D and A593E amino acid substitution mutations. The disclosure also provides use of such AAVs or VLPs in the manufacture of a medicament for delivering a cargo to a target cell and such AAVs or VLPs for use in delivering a cargo to a target cell.

In some aspects, the major capsid protein VP1, minor capsid protein VP2, and/or minor capsid protein VP3 comprise one or more further amino acid substitution mutations and/or comprise one or more amino acid insertions or deletions, e.g., comprise any of the features described in Section II(H), above.

The cell may be, e.g., an endothelial cell (e.g., a brain endothelial cell), a cancer cell, a central nervous system (CNS) cell, an eye cell, a retinal cell, a muscle cell, a stem cell, a heart cell, a lung cell, a skin cell, a kidney cell, and a liver cell. In some aspects, the target cell is an in vitro cell. In other aspects, the target cell is a cell in an organism (e.g., a mammal, e.g., a human).

### Capsids Comprising I451Y, S454I, and P504I Mutations

In another aspect, the disclosure features a method of delivering a cargo to a cell, the method comprising contacting a target cell with a modified AAV or virus-like particle (VLP) having a capsid comprising (a) a major capsid protein VP1 comprising I451Y, S454I, and/or P504I amino acid substitution mutations; (b) a minor capsid protein VP2 comprising I451Y, S454I, and/or P504I amino acid substitution mutations; and/or (c) a minor capsid protein VP3 comprising I451Y, S454I, and/or P504I amino acid substitution mutations; wherein the cargo is encapsulated by the AAV or VLP, wherein the amino acid substitution mutations are numbered relative to the AAV9 major capsid protein VP1 (SEQ ID NO: 1), and wherein the AAV or VLP has an increased rate of transduction into a target cell relative to an AAV or VLP having a capsid (a) not comprising any of the indicated amino acid substitution mutations or (b) comprising fewer of the indicated amino acid substitution mutations than the modified AAV or VLP of interest.

In another aspect, the disclosure features a method of delivering a cargo to a cell, the method comprising contacting a target cell with a modified AAV or virus-like particle (VLP) having a capsid comprising (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations; wherein the cargo is encapsulated by the AAV or VLP, wherein the amino acid substitution mutations are numbered relative to the AAV9 major capsid protein VP1 (SEQ ID NO: 1), and wherein the AAV or VLP has an increased rate of transduction into a target cell relative to an AAV or VLP having a capsid (a) not comprising any of the indicated amino acid substitution mutations or (b) comprising fewer of the indicated amino acid substitution mutations than the modified AAV or VLP of interest. The disclosure also provides use of such AAVs or VLPs in the manufacture of a medicament for delivering a cargo to a target cell and such AAVs or VLPs for use in delivering a cargo to a target cell.

Thus, in some aspects, the disclosure provides use of a modified AAV in the manufacture of a medicament for delivering a cargo to a target cell, wherein the modified AAV has a capsid comprising (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations, wherein the cargo is encapsulated by the AAV, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some aspects, the disclosure provides a modified AAV for use in delivering a cargo to a target cell, wherein the modified AAV has a capsid comprising (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations, wherein the cargo is encapsulated by the AAV, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV of interest.

In some aspects, the AAV or VLP is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV is AAV1, AAV2, or AAV9. Further serotypes that may be used in the invention are provided, e.g., in Section II(F), above.

In another aspect, the disclosure features a method of delivering a cargo to a cell, the method comprising contacting a target cell with a modified AAV or VLP having a capsid comprising: (a) a major capsid protein VP1 comprising I451Y, S454I, and P504I amino acid substitution mutations; (b) a minor capsid protein VP2 comprising I451Y, S454I, and P504I amino acid substitution mutations; and/or (c) a minor capsid protein VP3 comprising I451Y, S454I, and P504I amino acid substitution mutations, wherein the cargo is encapsulated by the AAV or VLP; wherein the AAV or VLP is AAV9 or a variant thereof and the amino acid substitution mutations are numbered relative to the AAV9 major capsid protein VP1 (SEQ ID NO: 1), and wherein the AAV or VLP has an increased rate of transduction into a target cell relative to an AAV9 or VLP derivative thereof having a capsid (a) not comprising any of the indicated amino acid substitution mutations or (b) comprising fewer of the indicated amino acid substitution mutations than the modified AAV or VLP of interest. The disclosure also provides use of such AAVs or VLPs in the manufacture of a medicament for delivering a cargo to a target cell and such AAVs or VLPs for use in delivering a cargo to a target cell.

In some aspects, (a) the major capsid protein VP1 further comprises an E500P amino acid substitution mutation; (b) the minor capsid protein VP2 further comprises an E500P amino acid substitution mutation; and/or (c) the minor capsid protein VP3 further comprises an E500P amino acid substitution mutation.

In some aspects, (a) the major capsid protein VP1 further comprises an E500R amino acid substitution mutation; (b) the minor capsid protein VP2 further comprises an E500R amino acid substitution mutation; and/or (c) the minor capsid protein VP3 further comprises an E500R amino acid substitution mutation. In some aspects, the disclosure features a method of delivering a cargo to a cell, the method comprising contacting a target cell with a modified AAV or VLP having a capsid comprising (a) a major capsid protein VP1 comprising I451Y, S454I, E500P, and P504I amino acid substitution mutations; (b) a minor capsid protein VP2 comprising I451Y, S454I, E500P, and P504I amino acid substitution mutations; and/or (c) a minor capsid protein VP3 comprising I451Y, S454I, E500P, and P504I amino acid substitution mutations; wherein the cargo is encapsulated by the AAV or VLP, wherein the amino acid substitution mutations are numbered relative to the AAV9 major capsid protein VP1 (SEQ ID NO: 1), and wherein the AAV or VLP has an increased rate of transduction into a target cell relative to an AAV or VLP having a capsid not comprising any of the indicated amino acid substitution mutations.

In some aspects in which the modified AAV or VLP comprises three of the four I451Y, S454I, E500P, and P504I amino acid substitution mutations or three of the four I451Y, S454I, E500R, and P504I amino acid substitution mutations, the modified AAV or VLP has an increased rate of transduction into a target cell relative to a control AAV or VLP having a capsid that comprises only one or only two of the indicated amino acid substitution mutations. In some aspects, the modified AAV or VLP comprises I451Y, S454I, and P504I amino acid substitution mutations and the modified AAV or VLP has an increased rate of transduction into a target cell relative to a control AAV or VLP having a capsid that comprises only one or only two of the indicated amino acid substitution mutations. In some aspects in which the modified AAV or VLP comprises all four of the I451Y, S454I, E500P, and P504I amino acid substitution mutations or all four of the I451Y, S454I, E500R, and P504I amino acid substitution mutations, the modified AAV or VLP has an increased rate of transduction into a target cell relative to a control AAV or VLP having a capsid that comprises only one, only two, or only three of the indicated amino acid substitution mutations.

In some aspects, the AAV or VLP is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV is AAV1, AAV2, or AAV9. In some aspects, the AAV or VLP is AAV9 or a variant thereof.

In some aspects, the cargo of the AAV or VLP is a nucleic acid, e.g., a single-stranded RNA (ssRNA). In some aspects, the cargo is a therapeutic nucleic acid. In some aspects, the cargo is a nucleic acid that is useful as a gene therapy. In some aspects, the cargo of the AAV or VLP is an antisense oligonucleotide or an siRNA. In some aspects, the inverted terminal repeat (ITR) sequences of the AAV genome are retained for packaging, and the viral genes are replaced with a sequence comprising or encoding the cargo.

In some aspects, the major capsid protein VP1, minor capsid protein VP2, and/or minor capsid protein VP3 comprise one or more further amino acid substitution mutations and/or comprise one or more amino acid insertions or deletions, e.g., comprise any of the features described in Section II(H), above.

The cell may be, e.g., an endothelial cell (e.g., a brain endothelial cell), a cancer cell, a central nervous system (CNS) cell, an eye cell, a retinal cell, a muscle cell, a stem cell, a heart cell, a lung cell, a skin cell, a kidney cell, and a liver cell. In some aspects, the target cell is an in vitro cell. In other aspects, the target cell is a cell in an organism (e.g., a mammal, e.g., a human).

### Alternative Amino Acid Substitutions at Position I451

In some embodiments of any of the aspects provided herein, the modified AAV or VLP may comprise an I451Y or I451M amino acid substitution mutation instead of the I451T amino acid substitution mutation.

### Alternative Amino Acid Substitution at Position S454

In some embodiments of any of the aspects provided herein, the modified AAV or VLP may comprise a S454F amino acid substitution mutation instead of the S454I amino acid substitution mutation.

### Alternative Amino Acid Substitutions at Position P504

In some embodiments of any of the aspects provided herein, the modified AAV or VLP may comprise a P504T or P504V amino acid substitution mutation instead of the P504I amino acid substitution mutation.

### Further Capsid Mutations

In another aspect, the disclosure features a method of delivering a cargo to a cell, the method comprising contacting a target cell with a modified AAV or virus-like particle (VLP) having a capsid comprising (a) a major capsid protein VP1; (b) a minor capsid protein VP2; and/or (c) a minor capsid protein VP3 comprising one or more of an I451T, I451Y, or I451M amino acid substitution mutation; an S454I or S454F amino acid substitution mutation; an A472F, A472H, A472K, A472N, A472W, or A472Y amino acid substitution mutation; a P504I, P504T, or P504V amino acid substitution mutation; an E500P or E500R amino acid substitution mutation; an A273N amino acid substitution mutation; a Q387K amino acid substitution mutation; an S263A or S263Y amino acid substitution mutation, and a W503H amino acid substitution mutation, wherein the cargo is encapsulated by the AAV or VLP, wherein the amino acid substitution mutations are numbered relative to the AAV9 major capsid protein VP1 (SEQ ID NO: 1), and wherein the AAV or VLP has an increased rate of transduction into a target cell relative to an AAV or VLP having a capsid (a) not comprising any of the indicated amino acid substitution mutations or (b) comprising fewer of the indicated amino acid substitution mutations than the modified AAV or VLP of interest. The disclosure also provides use of such AAVs or VLPs in the manufacture of a medicament for delivering a cargo to a target cell and such AAVs or VLPs for use in delivering a cargo to a target cell.

Thus, in some aspects, the disclosure provides use of a modified AAV in the manufacture of a medicament for delivering a cargo to a target cell, wherein the modified AAV has a capsid comprising (a) a major capsid protein VP1; (b) a minor capsid protein VP2; and/or (c) a minor capsid protein VP3 comprising one or more of an I451T, I451Y, or I451M amino acid substitution mutation; an S454I or S454F amino acid substitution mutation; an A472F, A472H, A472K, A472N, A472W, or A472Y amino acid substitution mutation; a P504I, P504T, or P504V amino acid substitution mutation; an E500P or E500R amino acid substitution mutation; an A273N amino acid substitution mutation; a Q387K amino acid substitution mutation; an S263A or S263Y amino acid substitution mutation, and a W503H amino acid substitution mutation, wherein the cargo is encapsulated by the AAV, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV or VLP of interest.

In some aspects, the disclosure provides a modified AAV for use in delivering a cargo to a target cell, wherein the modified AAV has a capsid comprising (a) a major capsid protein VP1; (b) a minor capsid protein VP2; and/or (c) a minor capsid protein VP3 comprising one or more of an I451T, I451Y, or I451M amino acid substitution mutation; an S454I or S454F amino acid substitution mutation; an A472F, A472H, A472K, A472N, A472W, or A472Y amino acid substitution mutation; a P504I, P504T, or P504V amino acid substitution mutation; an E500P or E500R amino acid substitution mutation; an A273N amino acid substitution mutation; a Q387K amino acid substitution mutation; an S263A or S263Y amino acid substitution mutation, and a W503H amino acid substitution mutation, wherein the cargo is encapsulated by the AAV, wherein the amino acid substitution mutations are numbered relative to the AAV9 major capsid protein VP1 (SEQ ID NO: 1), and wherein the transduction affinity of the modified AAV is improved relative to the transduction efficiency of an AAV having a capsid (a) not comprising any of the indicated amino acid substitution mutations or (b) comprising fewer of the indicated amino acid substitution mutations than the modified AAV or VLP of interest.

In some aspects, the AAV or VLP comprises two, three, four, five, six, or seven, eight, or nine of the listed amino acid substitution mutations. For example, in some aspects, the AAV or VLP has a capsid comprising (a) a major capsid protein VP1; (b) a minor capsid protein VP2; and/or (c) a minor capsid protein VP3 comprising I451T and A472H, I451Y and A472H, S454I and A472H, A472H and P504I, A472H and P504T, A472H and E500R, A273N and A472H, A472H and E500P, Q387K and A472H, A273N and Q387K, A273N and E500R, or Q387K and E500R amino acid substitution mutations. In some aspects, the AAV or VLP has a capsid comprising (a) a major capsid protein VP1; (b) a minor capsid protein VP2; and/or (c) a minor capsid protein VP3 comprising Q387K, A472H, and E500P; A273N, A472H, and E500P; I451Y, S454I, and E500P; or A273N, Q387K, and E500R amino acid substitution mutations. In some aspects, the AAV or VLP has a capsid comprising (a) a major capsid protein VP1; (b) a minor capsid protein VP2; and/or (c) a minor capsid protein VP3 comprising I451Y, S454I, E500P, and P504I; I451Y, S454I, E500P, and P504T; or A273N, Q387K, A472H, and E500P amino acid substitution mutations.

In some aspects, the AAV or VLP is of a serotype that interacts with the AAV receptor (AAVR) (e.g., AAV1, AAV2, AAV3, AAV6, AAV7, AAV8, AAV9, AAV10, AAV11, or AAV12). In some aspects, the AAV is AAV1, AAV2, or AAV9.

In some aspects, the cargo of the AAV or VLP is a nucleic acid, e.g., a single-stranded RNA (ssRNA). In some aspects, the cargo is a therapeutic nucleic acid. In some aspects, the cargo is a nucleic acid that is useful as a gene therapy. In some aspects, the cargo of the AAV or VLP is an antisense oligonucleotide or an siRNA. In some aspects, the inverted terminal repeat (ITR) sequences of the AAV genome are retained for packaging, and the viral genes are replaced with a sequence comprising or encoding the cargo.

In some aspects, the major capsid protein VP1, minor capsid protein VP2, and/or minor capsid protein VP3 comprise one or more further amino acid substitution mutations and/or comprise one or more amino acid insertions or deletions, e.g., comprise any of the features described in Section II(H), above.

The cell may be, e.g., an endothelial cell (e.g., a brain endothelial cell), a cancer cell, a central nervous system (CNS) cell, an eye cell, a retinal cell, a muscle cell, a stem cell, a heart cell, a lung cell, a skin cell, a kidney cell, and a liver cell. In some aspects, the target cell is an in vitro cell. In other aspects, the target cell is a cell in an organism (e.g., a mammal, e.g., a human).

### D. Methods of Delivery

The modified AAVs and VLPs provided herein can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The modified AAVs described herein can also be administered systemically or locally. The method of administration can vary depending on various factors (e.g., the compound or composition being administered and the severity of the condition, disease, or disorder being treated). Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein. In some aspects, the modified AAVs and/or VLPs provided herein are administered to a cell in an in vitro system.

A modified AAV or VLP described herein (and any additional therapeutic agent) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The modified AAV or VLP need not be, but is optionally formulated with and/or administered concurrently with, one or more agents currently used to prevent or treat the disorder in question. The effective amount of such other agents depends on the amount of the modified AAV present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

### IV. Methods of Determining AAV Capsid Affinity

In another aspect, the disclosure features a method for determining the affinity of an adeno-associated virus (AAV) capsid for a query protein, the method comprising (a) providing a virus-like particle (VLP) having a capsid consisting essentially of (e.g., consisting of) the VP3 subunit of the AAV capsid (or a variant thereof) (e.g., having a capsid not comprising VP1 or VP2); (b) contacting the VLP of step (a) with the query protein under conditions permitting the binding of the query protein and the VLP; and (c) quantifying the strength of the interaction between the query protein and the VLP, thereby determining the affinity of the AAV capsid for the query protein.

In some aspects, the VP3 subunit of the AAV capsid in the VLP is a variant VP3 subunit, e.g., a VP3 subunit comprising one or more amino acid insertions, deletions, and/or substitution mutations relative to the sequence of a wild-type VP3 subunit of the AAV capsid (e.g., relative to a wild-type AAV1, AAV2, AAV3, AAV4, AAV5, AAV6, AAV7, AAV8, AAV9, AAV10, AAV11, or AAV12 VP3 sequence). For example, in some instances, the VP3 subunit comprises one or more amino acid substitution mutations relative to the sequence of a wild-type VP3 subunit of the AAV capsid. In some aspects, the method comprises comparing the affinity for the query protein of a VLP comprising a variant VP3 subunit to that of a VLP comprising a wild-type VP3 subunit, thereby determining whether the variation(s) (e.g., amino acid insertions, deletions, and/or substitution mutations) affect (e.g., increase or decrease) the affinity of the AAV capsid for the query protein.

The AAV may be of any serotype. In some aspects, the serotype of the AAV is AAV1, AAV2, AAV3, AAV4, AAV5, AAV6, AAV7, AAV8, AAV9, AAV10, AAV11, or AAV12. Further serotypes that may be used in the invention are provided, e.g., in Section II(F), above.

Exemplary amino acid substitution mutations that may be assessed using the method include, but are not limited to an A273N, Q387K, A472H, or E500R amino acid substitution mutation numbered relative to the AAV9 major capsid protein VP1 (SEQ ID NO: 1) (e.g., A273N, Q387K, A472H, and/or E500R mutations in the VP3 subunit of AAV9) and T592D or A593E amino acid substitution mutations numbered relative to the AAV2 major capsid protein VP1 (SEQ ID NO: 2) (e.g., T592D and/or A593E mutations in the VP3 subunit of AAV2).

In some aspects, the VLP is immobilized on a solid surface (e.g. a sensor chip) and is contacted with the query protein, e.g., in soluble format. In some aspects, the solid surface comprises an antibody (e.g., a single-domain antibody) or antigen-binding fragment thereof (e.g., a Fab, Fab′, Fab′-SH, Fv, single chain variable fragment (scFv), or (Fab′)₂ fragment) having affinity for the VLP. In some aspects, the antibody or antigen-binding fragment thereof binds to the VP3 subunit of the AAV capsid.

In some aspects, VLPs are generated by co-transfecting a host cell (e.g., a mammalian cell, e.g., a HEK293 cell) with a first plasmid containing only the wild-type VP3 subunit of an AAV capsid and a second plasmid plasmid encoding the wild-type assembly-activating protein (AAP) of the AAV. In some aspects, the VP3 subunit and the AAP are derived from the same AAV serotype. In some aspects, the plasmids are transfected at a molar ratio of 5:1 (VP3 plasmid:AAP plasmid). After an appropriate period of growth (e.g., 7 days), VLPs may be harvested from the host cells (e.g., from conditioned media and/or from lysate of pelleted host cells).

In some aspects, the VLPs are not purified. For example, in some aspects, the VLP is produced in a cell line, immobilizing the VLP on the solid surface comprises contacting the solid surface with a culture medium comprising the VLP. In some aspects, the culture medium comprises lysate of the cell line from which the VLP is produced (e.g., cells of the cell line from which the VLP is produced are lysed, and the VLPs are not separated from the lysate of the cells prior to immobilizing the VLP on the solid surface.

Quantifying the strength of the interaction between the query protein and the VLP may be performed using any appropriate assay for assessing in vitro protein-protein interaction. In some aspects, the quantifying is performed using biolayer interferometry (BLI) and/or surface plasmon resonance (SPR). In some aspects, an initial BLI assay is performed, and an SPR assay is used to confirm or validate the results of the BLI assay and/or to quantify binding kinetics.

### VI. Examples

### Example 1. AAV Capsid Engineering for Improved Transduction

### A. Background

Adeno-associated viruses (AAVs) are non-pathogenic single-stranded DNA (ssDNA) viruses that may be used, e.g., as vectors for gene therapy. AAVs have a non-enveloped icosahedral capsid composed of major capsid protein VP1, minor capsid protein VP2, and minor capsid protein VP3, which are encoded by overlapping genes.

An important factor in the use of AAVs is mitigation of their immunogenicity in the subject. Factors affecting immunogenicity of an AAV include pre-existing humoral immunity of the subject to the AAV (the majority of the human population has already been exposed to at least one AAV serotype) and innate and adaptive immune responses. These immune responses have been shown to correlate with viral genome dose.

The experiments provided herein are based on the hypothesis that improving the affinity of the AAV capsid to the AAV receptor (AAVR) could increase transduction efficiency of the AAV. Increased transduction affinity might reduce dose requirements, potentially mitigating immunogenicity.

AAVR is critical for transduction of most AAV serotypes into the cell. AAVR rapidly traffics between the plasma membrane and the trans-Golgi network, and acts upstream of or at the stage of nuclear import of the AAV.

Although AAVR is required for transduction of AAVs, AAV attachment is independent of AAVR (Dudek et al., JVI, 92(7): e02213-17, 2018; Dudek et al., Mol Ther., 28(2): 367-381, 2020). Instead, AAV attachment to a host cell occurs through binding of the AAV to a primary receptor. The primary receptor is serotype-dependent, but is often related to glycans and heparan sulfate proteoglycans (HSPGs).

### B. Workflow for Screening AAV Virus-Like Particle (VLP) Mutants

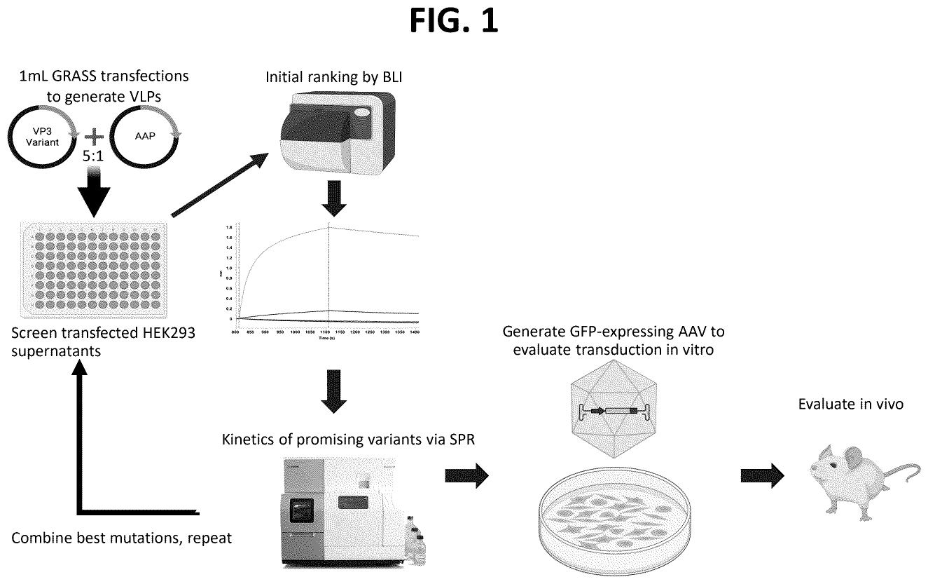

A method was developed for screening the affinity of AAV capsid variants to a target receptor using unpurified virus-like particles (VLPs), rather than full AAV virions (FIG. 1; image made with BioRender). The VLPs used in the method are composed of only VP3. This method retains the relevant exterior of the capsid and capsid assembly; however, the VLPs are easier to express and screen and are safer than using full virions. A sufficient quantity of VLPs for the experiments described herein can be collected from <1 mL of conditioned media. This minimal volume requirement is conducive to high-throughput screening.

Variant VP3 proteins (e.g., VP3 proteins comprising one or more amino acid substitution mutations of interest) were generated by introducing the desired mutations into a template plasmid containing only the wild-type VP3 subunit of an AAV capsid (e.g., AAV2 or AAV9). The resulting mutant plasmids were then co-transfected into mammalian cells (HEK293) with a separate plasmid encoding the wild-type assembly-activating protein (AAP) of the AAV (e.g., AAV2 or AAV9 AAP) at a molar ratio of 5:1 (VP3:AAP). Alternatively, the co-transfection may be transformed at a molar ratio of between 1:1 VP3:AAP to 20:1 VP3:AAP. After 7 days of growth, the cells and conditioned media were harvested. Either the conditioned media or lysates of the cell pellets can be used as sources of VLPs.

Supernatants of the transfected cells are screened, and an initial ranking of VP3 variants based on affinity to AAVR is determined using biolayer interferometry (BLI). Binding studies are performed using a fragment of AAVR comprising of the domains “PKD1” and “PKD2” (residues V305-V499, numbering starting from the initiating methionine of the full protein (SEQ ID NO: 3)), as these are the only domains known to interact with the AAV capsid. The AAVR binding kinetics of promising variants are determined using surface plasmon resonance (SPR). In some instances, promising mutations are combined, and the transfection and screening steps are repeated for VLPs comprising multiple mutations. GFP-expressing AAVs composed of VP1, VP2, and VP3 proteins comprising the mutation(s) of interest are then generated, and transduction efficiency is evaluated in vitro and in vivo.

To avoid having to purify the VLPs, an antibody, single-domain antibodies (sdABs) or fragments thereof (e.g., Fabs) can be immobilized as the ligand on the sensor surface and used to capture VLPs directly from the conditioned media or cell lysate. The captured VLPs can then be used for measuring the affinity for the analyte of interest (e.g., AAVR). Other in vitro protein-protein interaction methods may also be used for affinity analysis.

### C. Rational Substitutions Selected Based on AAVR/AAV2 Structure

Structures are publicly available for AAVR in complex with AAV1 (Zhang et al., Nat Commun, 10(1): 3760, 2019), AAV2 (Zhang et al., Nat. Microbiol., 4(4): 675-682, 2019), AAV5 (Zhang et al., Nat Commun, 10(1): 3760, 2019), and AAV9 (Xu et al., Mol Ther Methods Clin Dev, 26: 52-60, 2022). Mutations have been made to AAVR at its interface with the AAV capsid. Most of these mutations reduce both affinity of AAVR for the AAV capsid and transduction efficiency of AAV into cells.

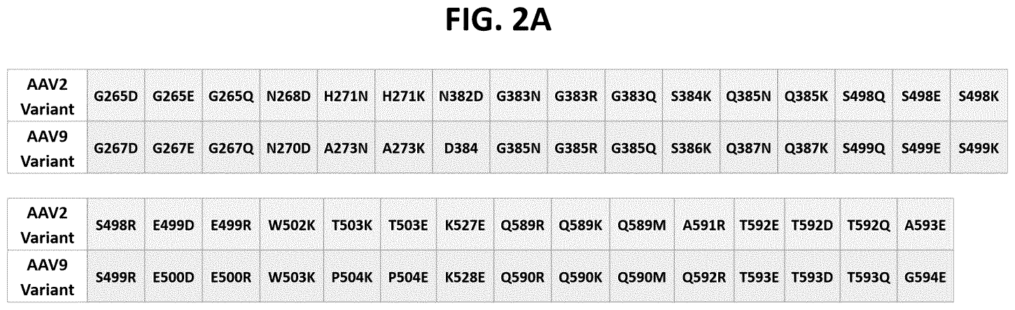

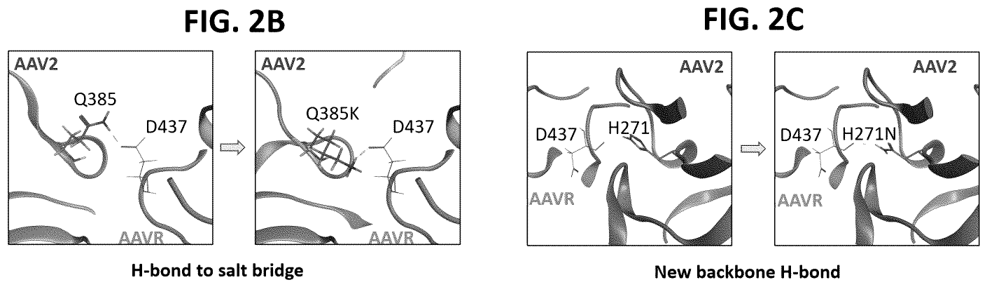

31 AAV capsid variants with mutations across 16 residues at the capsid/AAVR interface were designed for both AAV2 and AAV9 (FIG. 2A).

The variants tested in AAV2 were G265D, G265E, G265Q, N268D, H271N, H271K, N382D, G383N, G383R, G383Q, S384K, Q385N, Q385K, S498Q, S498E, S498K, S498R, E499D, E499R, W502K, T503K, T503E, K527E, Q589R, Q589K, Q589M, A591R, T592E, T592D, T592Q, and A593E (FIG. 2A).

The variants tested in AAV9 were G267D, G267E, G267Q, N270D, A273N, A273K, G385N, G385R, G385Q, S386K, Q387N, Q387K, S499Q, S499E, S499K, S499R, E500D, E500R, W503K, P504K, P504E, K528E, Q590R, Q590K, Q590M, Q592R, T593E, T593D, T593Q, and G594E (FIG. 2A).

The Q385K mutation in AAV2 was designed to replace a hydrogen bond with a salt bridge (FIG. 2B). The H271N mutation in AAV2 was designed to create a new backbone hydrogen bond (FIG. 2C).

### D. AAV2 VLP Variants Having Improved AAVR Affinity

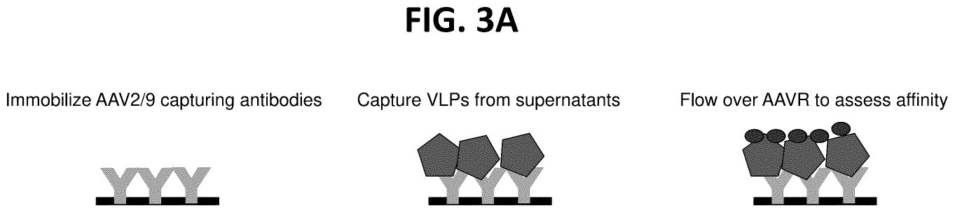

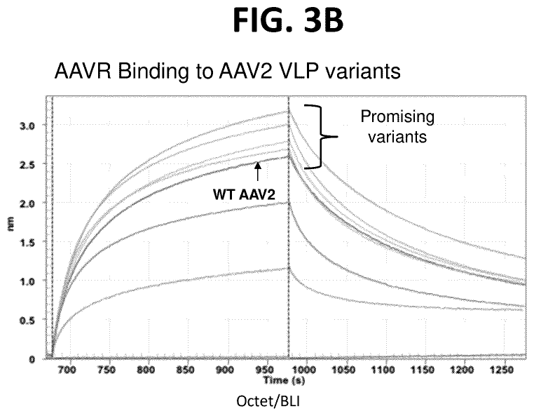

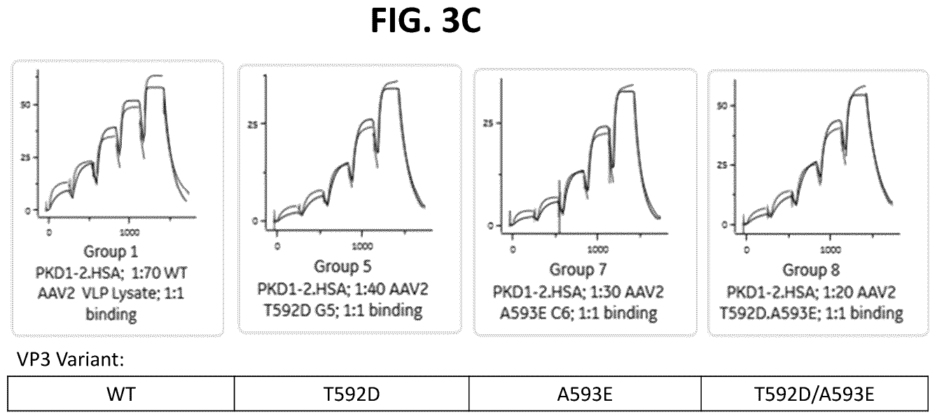

FIG. 3A shows a workflow for assessing affinity of VLPs for AAVR using BLI. AAV2 and AAV9 capturing antibodies are immobilized on a surface. VLPs from supernatant are captured on the antibodies, and AAVR is flowed over the surface to assess affinity.

T592D, A593E, and the combination of T592D and A593E (T592D/A593E) were identified as promising variants in AAV2 based on increased binding relative to a wild-type AAV2 VLP control, as shown in a BLI assay (FIG. 3B).

Affinity of the T592D, A593E, and T592D/A593E variants for AAVR was further assessed using SPR (FIG. 3C and Table 1). The T592D variant had 5× greater affinity for AAVR than a wild-type control. The A593E variant had about 4× greater affinity for AAVR than a wild-type control. The T592D/A593E variant had about 6× greater affinity for AAVR than a wild-type control.

TABLE 1
Affinity of AAV2 VP3 variants for AAVR

| | VP3 Variant: | | | |
|---|---|---|---|---|
| | Wild-type | | | |
| | AAV2 VLP | | | T592D/ |
| | (WT) | T592D | A593E | A593E |
| kₐ(1/Ms): | 1.51 × 10⁴ | 1.91 × 10⁴ | 2.16 × 10⁴ | 1.96 × 10⁴ |
| k_{d}(1/s): | 3.11 × 10⁻² | 7.97 × 10⁻³ | 1.11 × 10⁻² | 6.52 × 10⁻³ |
| KD (μM): | 2.1 | 0.42 | 0.52 | 0.33 |
| Fold change | N/A | 5X | ~4X | ~6X |
| vs WT: | | | | |

### E. AAV9 VLP Variants Having Improved AAVR Affinity

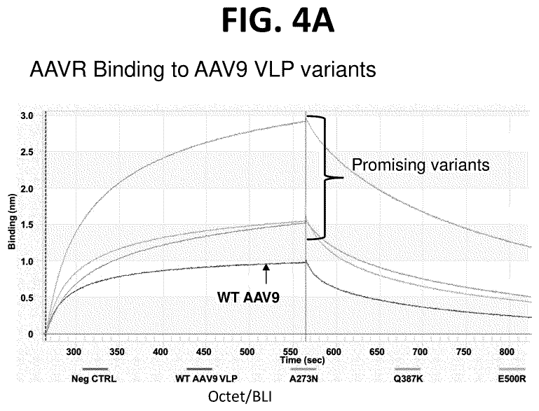

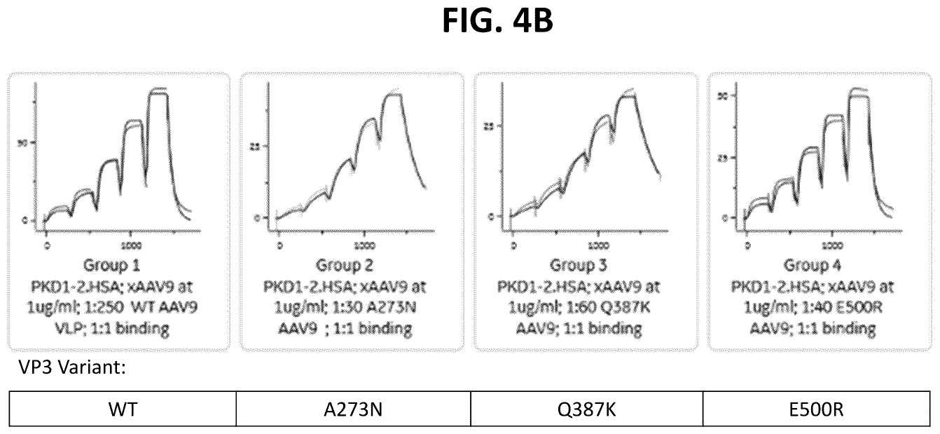

A273N, Q387K, and E500R were identified as promising variants in AAV9 based on increased binding relative to a wild-type AAV9 VLP control, as shown in a BLI assay (FIG. 4A).

Affinity of the A273N, Q387K, and E500R variants for AAVR was further assessed using SPR (FIG. 4B and Table 2; n=1). The A273N variant had 3.6× greater affinity for AAVR than a wild-type control. The Q387K variant had 14.9× greater affinity for AAVR than a wild-type control. The E500R variant had 1.6× greater affinity for AAVR than a wild-type control.

TABLE 2
Affinity of AAV9 VP3 variants for
AAVR (data from single replicate)

| | VP3 Variant: | | | |
|---|---|---|---|---|
| | Wild-type | | | |
| | AAV9 VLP | | | |
| | (WT) | A273N | Q387K | E500R |
| kₐ(1/Ms): | 4.89 × 10³ | 5.27 × 10³ | 1.60 × 10⁴ | 7.77 × 10³ |
| k_{d}(1/s): | 1.64 × 10⁻² | 4.87 × 10⁻³ | 3.60 × 10⁻³ | 1.66 × 10⁻² |
| KD (μM): | 3.36 | 0.93 | 0.23 | 2.14 |
| Fold change | N/A | 3.6X | 14.9X | 1.6X |
| vs WT: | | | | |

In a second SPR analysis (n=3) (Table 3), the A273N variant had 2.5× greater affinity for AAVR than a wild-type control; the Q387K variant had 15.6× greater affinity for AAVR than a wild-type control; and the E500R variant had 1.7× greater affinity for AAVR than a wild-type control.

TABLE 3
Affinity of AAV9 VP3 variants for
AAVR (data from three replicates)

| | VP3 Variant: | | | |
|---|---|---|---|---|
| | Wild-type | | | |
| | AAV9 VLP | | | |
| | (WT) | A273N | Q387K | E500R |
| kₐ(1/Ms): | 6.55 × 10³ | 5.10 × 10³ | 2.54 × 10⁴ | 9.74 × 10³ |
| k_{d}(1/s): | 1.53 × 10⁻² | 4.70 × 10⁻³ | 3.82 × 10⁻³ | 1.36 × 10⁻² |
| KD (μM): | 2.34 | 0.92 | 0.15 | 1.39 |
| Fold change | N/A | 2.5X | 15.6X | 1.7X |
| vs WT: | | | | |

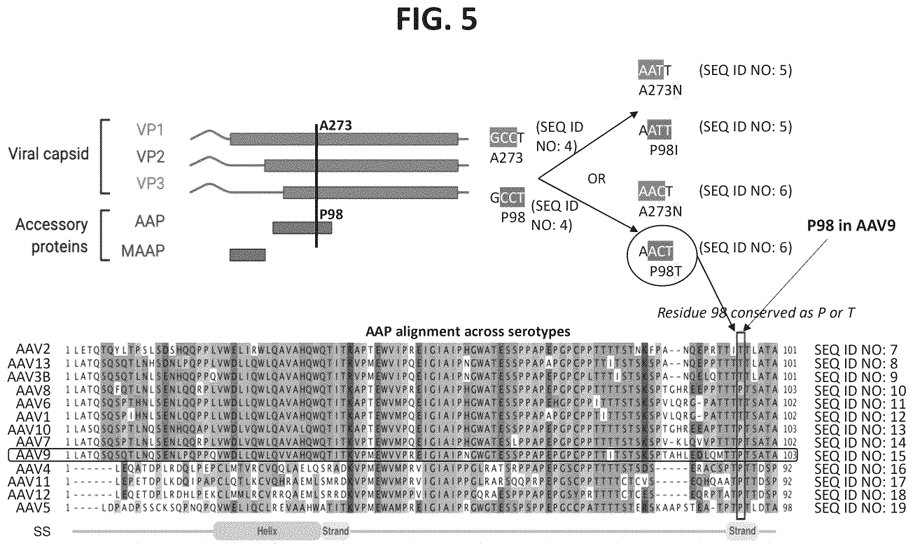

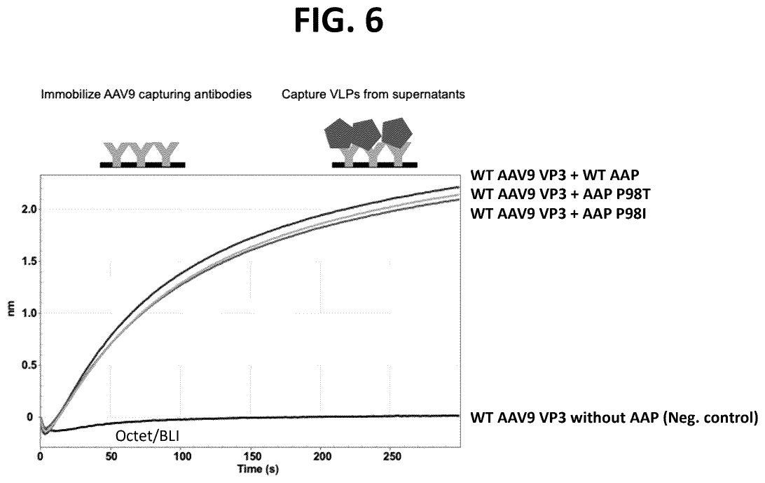

It is noted that the A273N substitution in the capsid would also alter the P98 codon in the AAV assembly-activating protein (AAP) (FIG. 5). AAV9 AAP tolerates both P98T and P981 substitutions: AAV9 VLPs generated with co-expressed AAP mutants showed similar capture levels from conditioned media on anti-AAV9 BLI biosensor (FIG. 6).

### F. AAV9 VLP Variant Demonstrates Increased Transduction In Vitro

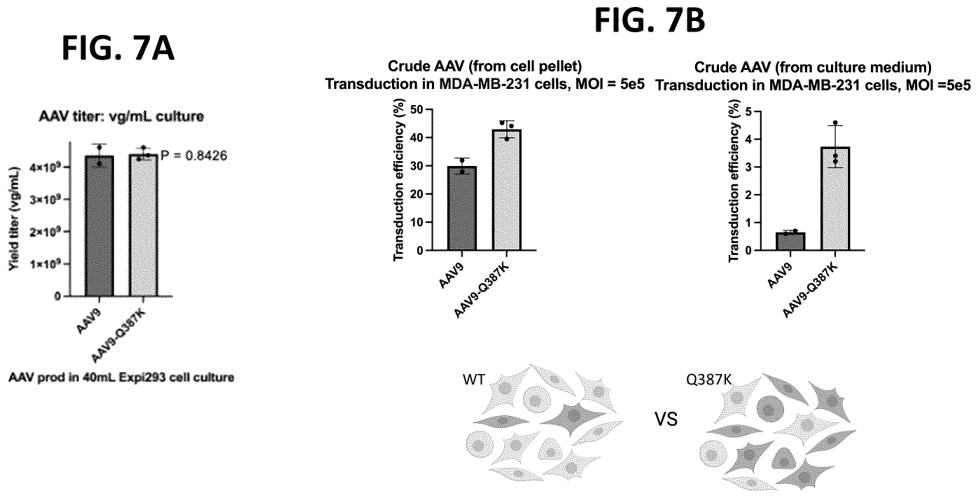

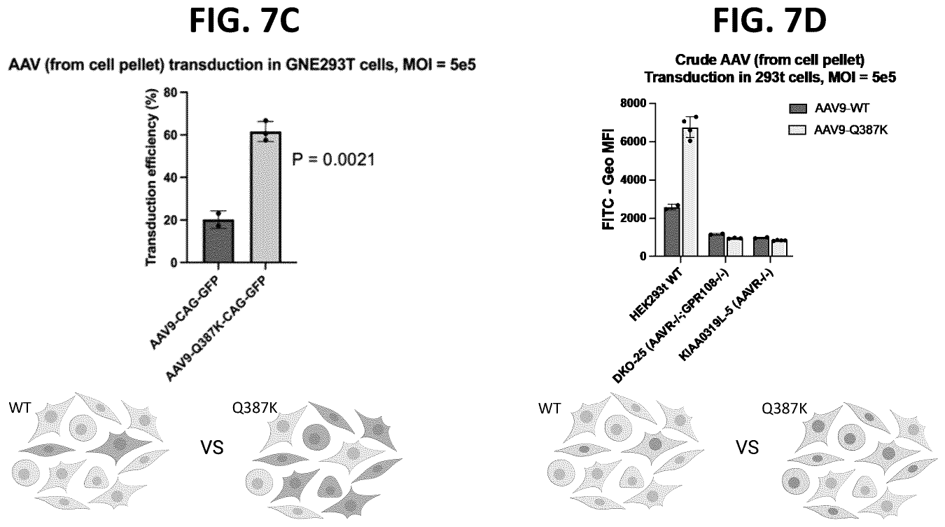

AAV9 AAVs comprising the Q387K variant produced similar titers to wild-type AAV9 (FIG. 7A). The percentage of cells transduced by the Q387K AAV9 variant increased by 1.5- to 4-fold in a breast cancer cell line (FIG. 7B) and by about 3-fold in HEK293 cells (293/tsA1609neo cells) (FIG. 7C). 293/tsA1609neo cells are described, e.g., in DuBridge et al., Mol Cell Biol, 7(1): 379-387, 1987). Expression of a transgene (GFP) in transduced HEK293 cells increased by ˜3-fold (FIG. 7D). This enhancement was dependent on AAVR (FIG. 7D).

### G. AAV9 VLP Variant Combinations have Up to 53× Stronger Affinity to AAVR

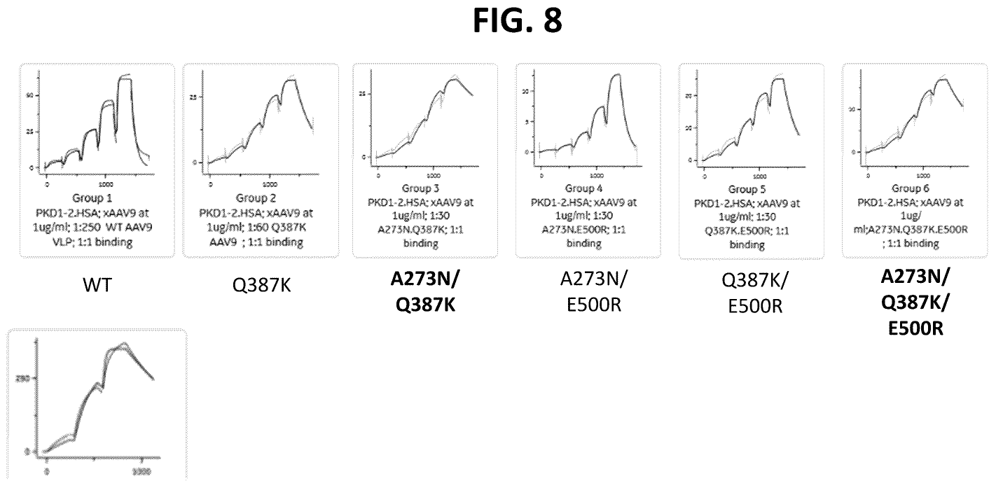

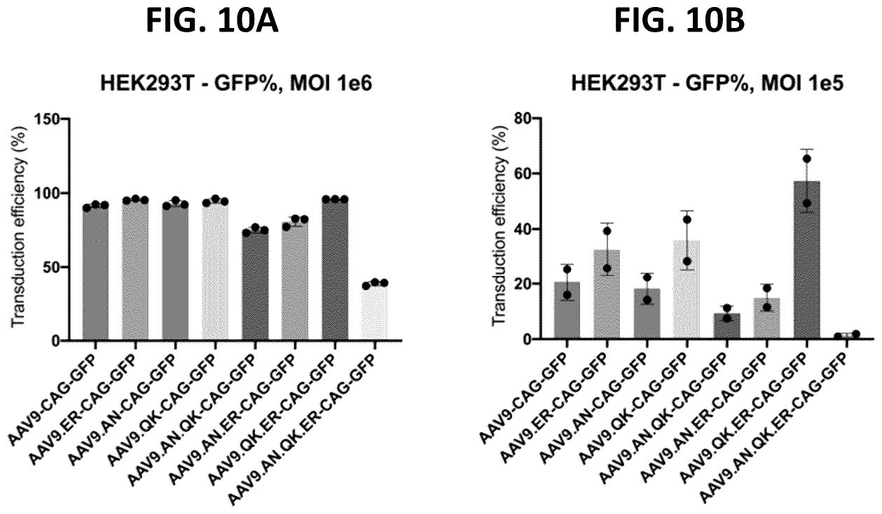

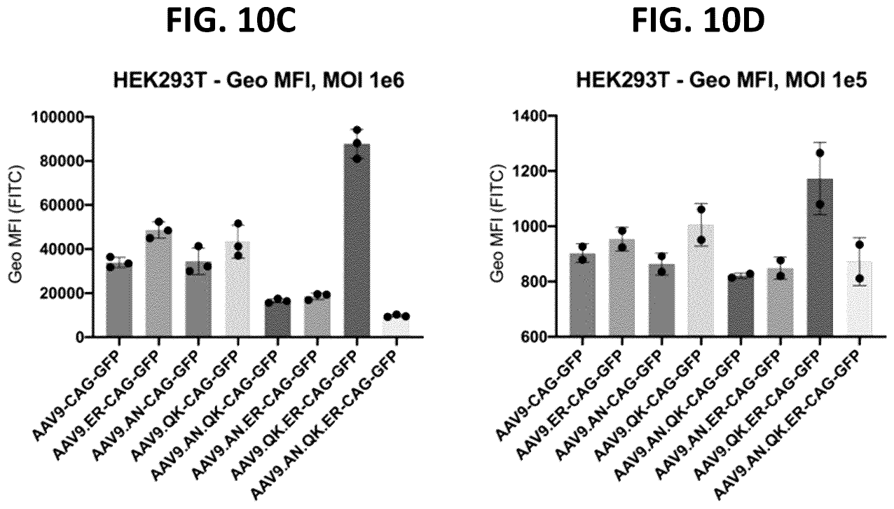

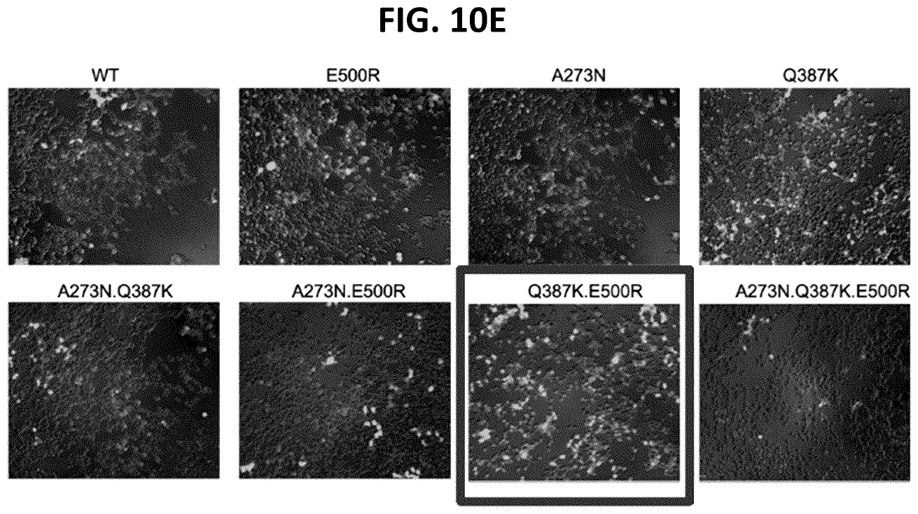

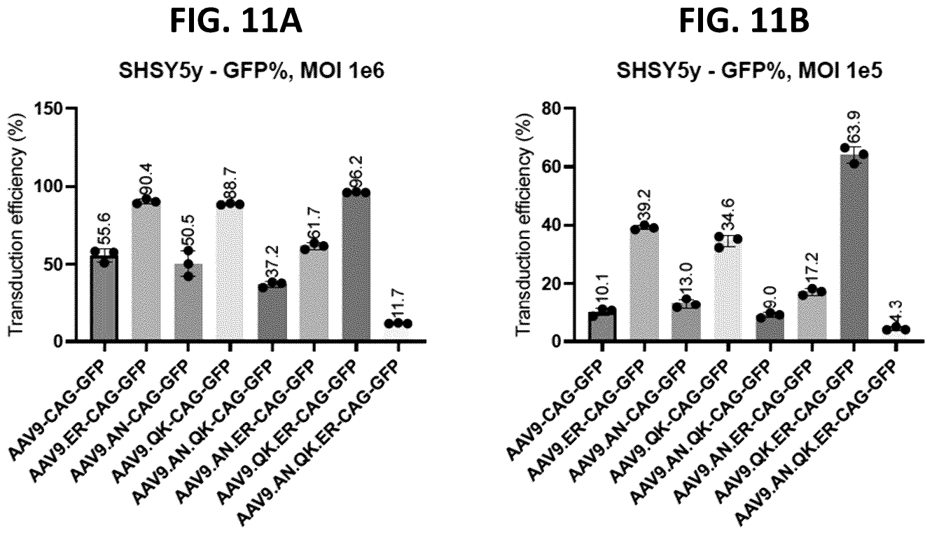

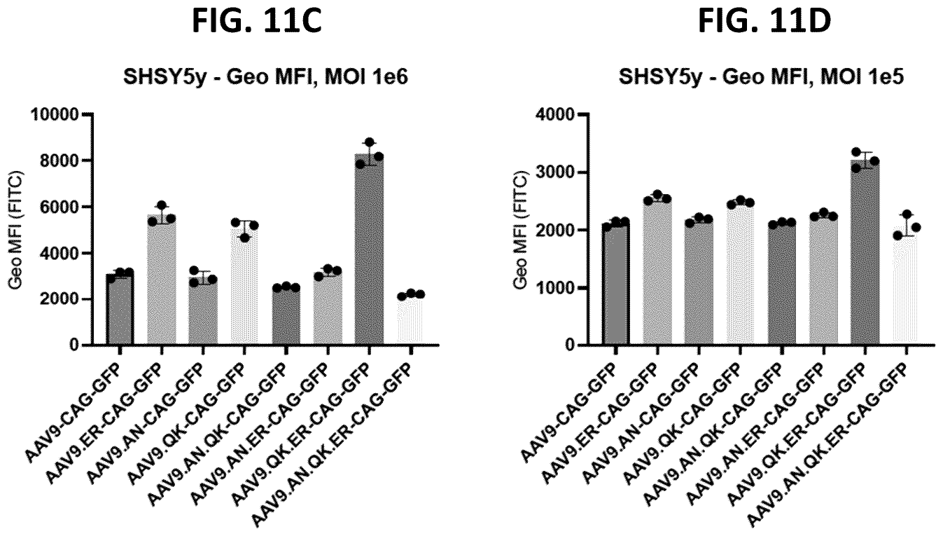

Combinations of the A273N, Q387K, and E500R amino acid substitution mutations in AAVR VLPs resulted in even higher affinities for AAVR (FIG. 8 and Tables 4 (n=1) and 5 (n=3)), with the highest affinity observed in VLPs comprising all three of the A273N, Q387K, and E500R amino acid substitution mutations. FIGS. 10A-10E and 11A-11D show the transduction efficiency of AAVs comprising E500R (“ER”), A273N (“AN”), Q387K (“QK”), A273N and Q387K (“AN.QK”), A273N and E500R (“AN.ER”), Q387K and E500R (“QK.ER”), or A273N, Q387K, and E500R (“AN.QK.ER”) in HEK293T and SH-SY5y (neuroblastoma) cells.

TABLE 4
Affinity of AAV9 VP3 variant combinations
for AAVR (data from single replicate)

| | VP3 Variant: | | | | | | |
|---|---|---|---|---|---|---|---|
| | | | A273N/ | A273N/ | Q387K/ | A273N/ | |
| | WT | Q387K | Q387K | E500R | E500R | Q387K/E500R | A472H |
| kₐ(1/Ms): | 3.18 × | 1.25 × | 8.39 × | 1.04 × | 1.57 × | 1.18 × | 5.63 × |
| | 10³ | 10⁴ | 10³ | 10⁴ | 10⁴ | 10⁴ | 10³ |
| k_{d}(1/s): | 1.35 × | 3.12 × | 8.23 × | 4.03 × | 3.00 × | 9.62 × | 1.17 × |
| | 10⁻² | 10⁻³ | 10⁻⁴ | 10⁻³ | 10⁻³ | 10⁻⁴ | 10⁻³ |
| KD (μM): | 4.25 | 0.25 | 0.10 | 0.38 | 0.19 | 0.08 | 0.21 |
| Fold change | N/A | 17X | 42.5X | 11X | 22X | 53X | 16X |
| vs WT: | | | | | | | |

TABLE 5
Affinity of AAV9 VP3 variant combinations
for AAVR (data from three replicates)

| | VP3 Variant: | | | | | | |
|---|---|---|---|---|---|---|---|
| | | | A273N/ | A273N/ | Q387K/ | A273N/ | |
| | WT | Q387K | Q387K | E500R | E500R | Q387K/E500R | A472H |
| kₐ(1/Ms): | 6.55 × | 2.54 × | 2.81 × | 1.89 × | 2.61 × | 3.33 × | 8.52 × |
| | 10³ | 10⁴ | 10⁴ | 10⁴ | 10⁴ | 10⁴ | 10³ |
| k_{d}(1/s): | 1.53 × | 3.82 × | 4.20 × | 4.92 × | 2.53 × | 6.67 × | 1.64 × |
| | 10⁻² | 10⁻³ | 10⁻³ | 10⁻³ | 10⁻³ | 10⁻⁴ | 10⁻³ |
| KD (μM): | 2.34 | 0.15 | 0.15 | 0.26 | 0.1 | 0.02 | 0.19 |
| Fold change | N/A | 15.6X | 15.6X | 9X | 23X | 117X | 12.3X |
| vs WT: | | | | | | | |

### H. A472H VLP Variant

In order to identify AAV capsid residues important for the interaction of the capsid with AAVR, an alanine screen of AAV9 VP3 VLPs was performed across the following residues that are located at or near the interface with AAVR: S261-A273, L382-G390, N452-T460, A466-Q474, T491-N515, S526-L541, and A581-W595. Residues at the indicated positions were substituted with alanine. In cases in which the residue of interest was already alanine (e.g., A472), the alanine was substituted with a histidine, which has a large hydrophilic side chain to contrast the small hydrophobic sidechain of alanine. Mutants resulting from the screen were investigated for binding to AAVR using SPR. The expectation was that the alanine or histidine substitutions would result in a decreased affinity for AAVR. Unexpectedly, in the case of A472H, an improved affinity was observed.

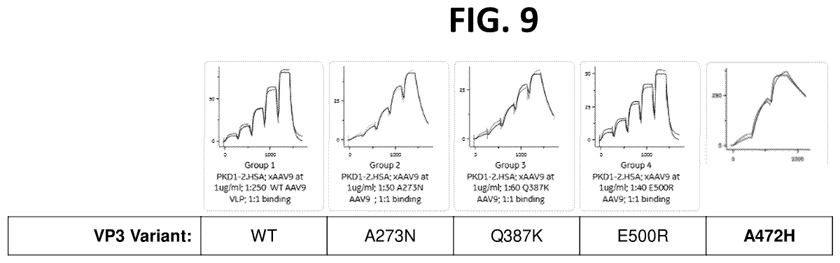

A472H was identified as a promising variant in AAV9 based on increased binding relative to a wild-type AAV9 VLP control. Affinity of the A273N, Q387K, E500R, and A472H variants for AAVR was assessed using SPR (FIG. 9 and Table 6). The A472H variant had 16× greater affinity for AAVR than a wild-type control.

TABLE 6
Affinity of AAV9 VP3 variant combinations for AAVR

| VP3 Variant: | WT | A273N | Q387K | E500R | A472H |
|---|---|---|---|---|---|
| kₐ(1/Ms): | 4.89 × 10³ | 5.27 × 10³ | 1.60 × 10⁴ | 7.77 × 10³ | 5.63 × 10³ |
| k_{d}(1/s): | 1.64 × 10⁻² | 4.87 × 10⁻³ | 3.60 × 10⁻³ | 1.66 × 10⁻² | 1.17 × 10⁻³ |
| KD (μM): | 3.36 | 0.93 | 0.23 | 2.14 | 0.21 |
| Fold change | N/A | 3.6X | 14.9X | 1.6X | 16X |
| vs WT: | | | | | |

### Example 2. COSMO Workflow for AAV Capsid Engineering and Additional AAV Variants

### A. Method for Generating AAV9 VP3 Variants by COSMO

A high-throughput method (COSMO workflow) was developed to identify additional AAV9 VP3 variants that improve transduction efficiency.

The comprehensive substitution for multidimensional optimization (COSMO) workflow is a high-throughput site-directed mutagenesis protocol for antibody engineering applications (see Sampei et al., PloS ONE, 13(12): e0209509, 2018, which is incorporated herein by reference in its entirety).

The COSMO workflow was adapted for the high-throughput production of VLP capsid protein variants, and allows for mutagenesis and production of a large number of VLPs.

Analysis of AAV9 VP3 VLP interactions with AAVR was performed using SPR. VLPs were captured directly from cell media using Anti-AAV9-Biotin VHH (Thermo Scientific #7103332500) immobilized on a streptavidin (SA) sensor chip surface.

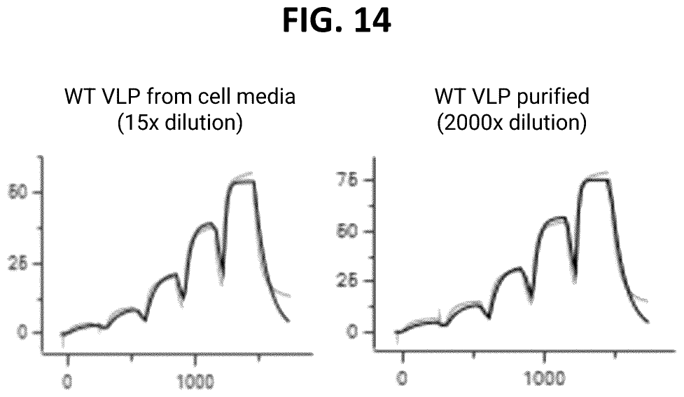

As shown in FIG. 14 and Table 7, SPR results using purified and unpurified VLPs were comparable.

TABLE 7
SPR results using purified and unpurified VLPs

VLP Variant:

| | WT VLP from cell media | WT VLP purified |
|---|---|---|
| | (15x dilution) | (2000x dilution) |
| kₐ(1/Ms): | 5.04 × 10³ | 6.34 × 10³ |
| k_{d}(1/s): | 9.23 × 10⁻³ | 1.01 × 10⁻² |
| KD (μM): | 1.83 | 1.59 |
| Rmax (RU) | 66 | 90.4 |

Mutants with increased binding affinity for AAVR were identified and further analyzed, as described below.

### Amino Acid Screening

Amino acid screening was performed across various residues at the interface between AAV9 VP3 and AAVR. Substitution mutations were introduced using the COSMO workflow described. Mutants were transferred to a 384-well plate and SPR was performed using the CARTERRA® LSA, using the capture method described.

### B. AAV9 VP3 Variants Generated by COSMO Workflow

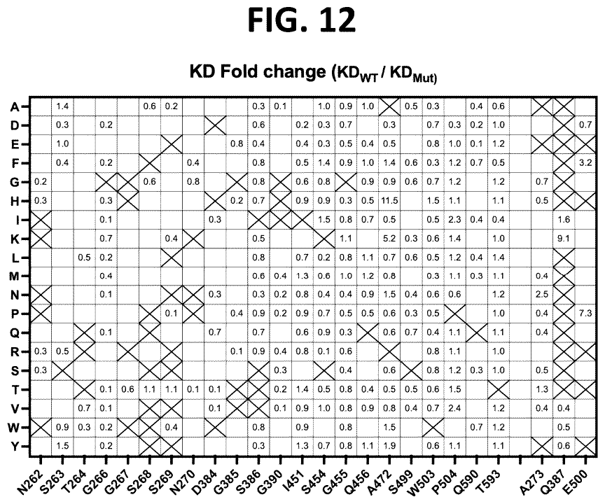

FIG. 12 shows the affinity for AAVR of VLPs generated by the COSMO workflow, presented as a fold change in K_{D} relative to a wild-type VLP. Sixteen of the tested mutants had at least a 1.5-fold increase in AAVR affinity (S263Y, S454I, A472H, A472K, A472N, A472W, A472Y, W503H, P504I, P504T, P504V, A273N, Q387I, Q387K, E500F, and E500P). A273, Q387, and E500 were residues explored during the rational design experiments described in Example 1, above. The A472H mutant was found to have a greater than ten-fold increase in affinity for AAVR relative to wild-type.

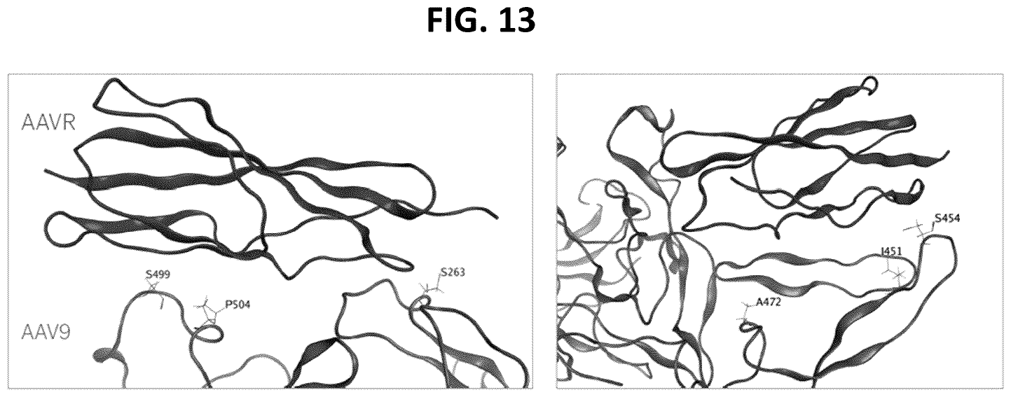

Variants with improved affinity for AAVR were identified at six locations (S263, I451, S454, A472, S499, and P504) shown in Table 8, below. Each of the variants shown in Table 8 has at least a 1.3-fold increase in K_{D} relative to wild-type. Ribbon diagrams showing the location of these residues in AAV9 (in complex with AAVR) are shown in FIG. 13. Additional SPR analyses of the affinity of the variants for AAVR are shown in Table 9; P504V, S499N, S263Y, I451K, and S454W were not analyzed due to low immobilization levels.

TABLE 8
CARTERRA ® analysis of affinity
of AAV9 VP3 variants for AAVR

| AAV9 Variant | K_{D} Fold Change |
|---|---|
| S263A | 1.4x |
| S263Y | 1.5x |
| I451M | 1.3x |
| I451T | 1.4x |
| I451Y | 1.3x |
| S454F | 1.3x |
| S454I | 1.5x |
| A472F | 1.4x |
| A472H | 11.5x |
| A472K | 5.2x |
| A472N | 1.5x |
| A472W | 1.5x |
| A472Y | 1.9x |
| W503H | 1.5x |
| P504I | 2.3x |
| P504T | 1.5x |
| P504V | 2.4x |

TABLE 9
SPR analysis of affinity of AAV9 VP3 variants and combinations thereof for AAVR

| AAV9 Variant | ka (1/Ms) | kd (1/s) | K_{D} (M) | K_{D} Fold Change |
|---|---|---|---|---|
| I451T | 5.82 × 10³ | 1.34 × 10⁻² | 2.30 × 10⁻⁶ | 1.0x |
| I451Y | 5.07 × 10³ | 9.03 × 10⁻³ | 1.78 × 10⁻⁶ | 1.3x |
| S454I | 7.11 × 10³ | 1.21 × 10⁻² | 1.70 × 10⁻⁶ | 1.4x |
| A472F | 7.26 × 10³ | 1.24 × 10⁻² | 1.71 × 10⁻⁶ | 1.4x |
| A472H | 8.52 × 10³ | 1.64 × 10⁻³ | 1.92 × 10⁻⁷ | 12.2x |
| A472K | 1.3210⁴ | 5.33 × 10⁻³ | 4.03 × 10⁻⁷ | 5.8x |
| A472N | 5.53 × 10³ | 1.06 × 10⁻² | 1.93 × 10⁻⁶ | 1.2x |
| A472W | 6.62 × 10³ | 1.03 × 10⁻² | 1.55 × 10⁻⁶ | 1.5x |
| A472Y | 6.48 × 10³ | 1.08 × 10⁻² | 1.67 × 10⁻⁶ | 1.4x |
| P504I | 1.02 × 10⁴ | 1.40 × 10⁻² | 1.37 × 10⁻⁶ | 1.7x |
| P504T | 8.00 × 10³ | 1.44 × 10⁻² | 1.80 × 10⁻⁶ | 1.3x |
| E500P | 1.19 × 10⁴ | 2.40 × 10⁻³ | 2.02 × 10⁻⁷ | 11.6x |
| I451Y, S454I, E500P | 1.81 × 10⁴ | 2.38 × 10⁻³ | 1.08 × 10⁻⁸ | 217x |
| I451Y, S454I, E500P, P504I | 2.25 × 10⁴ | 1.66 × 10⁻³ | 1.09 × 10⁻⁸ | 214x |
| I451Y, S454I, E500P, P504T | 2.13 × 10⁴ | 2.88 × 10⁻³ | 1.60 × 10⁻⁸ | 146x |
| I451T, A472H | 1.76 × 10⁴ | 9.27 × 10⁻⁴ | 5.26 × 10⁻⁸ | 44.5x |
| I451Y, A472H | 1.49 × 10⁴ | 6.21 × 10⁻⁴ | 4.18 × 10⁻⁸ | 56.0x |
| S454I, A472H | 1.79 × 10⁴ | 1.67 × 10⁻³ | 9.31 × 10⁻⁸ | 25.1x |
| A472H, P504I | 2.54 × 10⁴ | 8.80 × 10⁻⁴ | 3.46 × 10⁻⁸ | 67.5x |
| A472H, P504T | 2.30 × 10⁴ | 1.23 × 10⁻³ | 5.32 × 10⁻⁸ | 44.0x |
| A472H, E500R | 3.35 × 10⁴ | 4.46 × 10⁻³ | 1.33 × 10⁻⁷ | 17.6x |
| A273N, A472H | 1.35 × 10⁴ | 5.05 × 10⁻⁴ | 3.73 × 10⁻⁸ | 62.7x |
| A472H, E500P | 2.97 × 10⁴ | 5.10 × 10⁻⁴ | 1.72 × 10⁻⁸ | 136x |
| Q387K, A472H, E500P | 4.87 × 10⁵ | 3.38 × 10⁻⁴ | 6.94 × 10⁻¹⁰ | 3370x |
| A273N, A472H, E500P | 4.59 × 10⁴ | 2.36 × 10⁻⁴ | 5.14 × 10⁻⁹ | 455x |
| Q387K, A472H | 6.16 × 10⁴ | 4.30 × 10⁻⁴ | 6.98 × 10⁻⁹ | 335x |
| A273N, Q387K, A472H, E500P | 9.40 × 10⁴ | 3.96 × 10⁻⁵ | 4.21 × 10⁻¹⁰ | 5560x |
| A273N | 5.10 × 10³ | 4.70 × 10⁻³ | 9.2 × 10⁻⁷ | 2.5x |
| Q387K | 2.54 × 10⁴ | 3.82 × 10⁻³ | 1.5 × 10⁻⁷ | 15.6x |
| E500R | 9.74 × 10³ | 1.36 × 10⁻² | 1.39 × 10⁻⁶ | 1.7x |
| A273N, Q387K | 2.81 × 10⁴ | 4.20 × 10⁻³ | 1.5 × 10⁻⁷ | 15.6x |
| A273N, E500R | 1.89 × 10⁴ | 4.92 × 10⁻³ | 2.6 × 10⁻⁷ | 9.0x |
| Q387K, E500R | 2.61 × 10⁴ | 2.53 × 10⁻³ | 1.0 × 10⁻⁷ | 23x |
| A273N, Q387K, E500R | 3.33 × 10⁴ | 6.67 × 10⁻⁴ | 2.0 × 10⁻⁸ | 117x |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of improving transduction efficiency of an adeno-associated virus (AAV), the method comprising providing a modified AAV having a capsid comprising:
(I)
(a) a major capsid protein VP1 comprising an A472H amino acid substitution mutation;
(b) a minor capsid protein VP2 comprising an A472H amino acid substitution mutation; and/or
(c) a minor capsid protein VP3 comprising an A472H amino acid substitution mutation;
wherein the amino acid substitution mutations are numbered relative to the AAV9 major capsid protein VP1 (SEQ ID NO: 1);
(II)
(a) a major capsid protein VP1 comprising an E500R amino acid substitution mutation;
(b) a minor capsid protein VP2 comprising an E500R amino acid substitution mutation; and/or
(c) a minor capsid protein VP3 comprising an E500R amino acid substitution mutation;
wherein the AAV is AAV9 or a variant thereof and the amino acid substitution mutations are numbered relative to the AAV9 major capsid protein VP1 (SEQ ID NO: 1);
(III)
(a) a major capsid protein VP1 comprising T592D and A593E amino acid substitution mutations;
(b) a minor capsid protein VP2 comprising T592D and A593E amino acid substitution mutations; and/or
(c) a minor capsid protein VP3 comprising T592D and A593E amino acid substitution mutations;
wherein the AAV is AAV2 or a variant thereof and the amino acid substitution mutations are numbered relative to the AAV2 major capsid protein VP1 (SEQ ID NO: 2); or
(IV)
(a) a major capsid protein VP1 comprising I451Y, S454I, and P504I amino acid substitution mutations;
(b) a minor capsid protein VP2 comprising I451Y, S454I, and P504I amino acid substitution mutations; and/or
(c) a minor capsid protein VP3 comprising I451Y, S454I, and P504I amino acid substitution mutations;
wherein the AAV is AAV9 or a variant thereof and the amino acid substitution mutations are numbered relative to the AAV9 major capsid protein VP1 (SEQ ID NO: 1).

2-5. (canceled)

6. The method of claim 1, wherein:
(a) the major capsid protein VP1 comprises A273N, Q387K, A472H, and E500R amino acid substitution mutations;
(b) the minor capsid protein VP2 comprises A273N, Q387K, A472H, and E500R amino acid substitution mutations; and/or
(c) the minor capsid protein VP3 comprises A273N, Q387K, A472H, and E500R amino acid substitution mutations, wherein the amino acid substitution mutations are numbered relative to the AAV9 major capsid protein VP1 (SEQ ID NO: 1).

7-8. (canceled)

9. The method of claim 1, wherein the method comprises contacting a target cell with the modified AAV, thereby improving transduction efficiency.

10. The method of claim 1, wherein the transduction efficiency is improved relative to the transduction efficiency of an AAV having a capsid not comprising any of the indicated amino acid substitution mutations.

11. A method of delivering a cargo to a cell, the method comprising contacting a target cell with a modified AAV having a capsid comprising:
(I)
(a) a major capsid protein VP1 comprising an A472H amino acid substitution mutation;
(b) a minor capsid protein VP2 comprising an A472H amino acid substitution mutation; and/or
(c) a minor capsid protein VP3 comprising an A472H amino acid substitution mutation;
wherein the cargo is encapsulated by the AAV;
wherein the amino acid substitution mutations are numbered relative to the AAV9 major capsid protein VP1 (SEQ ID NO: 1);
and wherein the AAV has an increased rate of transduction into a target cell relative to an AAV having a capsid not comprising any of the indicated amino acid substitution mutations;
(II)
(a) a major capsid protein VP1 comprising an E500R amino acid substitution mutation;
(b) a minor capsid protein VP2 comprising an E500R amino acid substitution mutation; and/or
(c) a minor capsid protein VP3 comprising an E500R amino acid substitution mutation;
wherein the cargo is encapsulated by the AAV;
wherein the AAV is AAV9 or a variant thereof and the amino acid substitution mutations are numbered relative to the AAV9 major capsid protein VP1 (SEQ ID NO: 1);
and wherein the AAV has an increased rate of transduction into a target cell relative to an AAV9 having a capsid not comprising any of the indicated amino acid substitution mutations;
(III)
(a) a major capsid protein VP1 comprising T592D and A593E amino acid substitution mutations;
(b) a minor capsid protein VP2 comprising T592D and A593E amino acid substitution mutations; and/or
(c) a minor capsid protein VP3 comprising T592D and A593E amino acid substitution mutations;
wherein the cargo is encapsulated by the AAV;
wherein the AAV is AAV2 or a variant thereof and the amino acid substitution mutations are numbered relative to the AAV2 major capsid protein VP1 (SEQ ID NO: 2);
and wherein the AAV has an increased rate of transduction into a target cell relative to an AAV2 having a capsid not comprising either of the indicated amino acid substitution mutations; or
(IV)
(a) a major capsid protein VP1 comprising I451Y, S454I, and P504I amino acid substitution mutations;
(b) a minor capsid protein VP2 comprising I451Y, S454I, and P504I amino acid substitution mutations; and/or
(c) a minor capsid protein VP3 comprising I451Y, S454I, and P504I amino acid substitution mutations;
wherein the cargo is encapsulated by the AAV;
wherein the AAV is AAV9 or a variant thereof and the amino acid substitution mutations are numbered relative to the AAV9 major capsid protein VP1 (SEQ ID NO: 1);
and wherein the AAV has an increased rate of transduction into a target cell relative to an AAV9 having a capsid not comprising any of the indicated amino acid substitution mutations.

12-15. (canceled)

16. The method of claim 11, wherein:
(a) the major capsid protein VP1 comprises A273N, Q387K, A472H, and E500R amino acid substitution mutations;
(b) the minor capsid protein VP2 comprises A273N, Q387K, A472H, and E500R amino acid substitution mutations; and/or
(c) the minor capsid protein VP3 comprises A273N, Q387K, A472H, and E500R amino acid substitution mutations, wherein the amino acid substitution mutations are numbered relative to the AAV9 major capsid protein VP1 (SEQ ID NO: 1).

17-18. (canceled)

19. The method of claim 1, wherein the modified AAV has an increased affinity for AAV receptor (AAVR), has an increased rate of delivery to the nucleus of the target cell, and/or transduces the target cell at a lower dose relative to an AAV having a capsid that does not comprise any of the indicated amino acid substitution mutations.

20-25. (canceled)

26. The method of claim 9, wherein the target cell is an endothelial cell, a cancer cell, a central nervous system (CNS) cell, an eye cell, a retinal cell, a muscle cell, a stem cell, a heart cell, a lung cell, a skin cell, a kidney cell, or a liver cell.

27. The method of claim 26, wherein the endothelial cell is a brain endothelial cell.

28. The method of claim 1, wherein the major capsid protein VP1, minor capsid protein VP2, and/or minor capsid protein VP3 comprise one or more further amino acid substitution mutations and/or comprise one or more amino acid insertions or deletions.

29-34. (canceled)

35. An AAV having a capsid comprising:
(I)
(a) a major capsid protein VP1 comprising an A472H amino acid substitution mutation;
(b) a minor capsid protein VP2 comprising an A472H amino acid substitution mutation; and/or
(c) a minor capsid protein VP3 comprising an A472H amino acid substitution mutation;
wherein the amino acid substitution mutations are numbered relative to the AAV9 major capsid protein VP1 (SEQ ID NO: 1);
(II)
(a) a major capsid protein VP1 comprising an E500R amino acid substitution mutation;
(b) a minor capsid protein VP2 comprising an E500R amino acid substitution mutation; and/or
(c) a minor capsid protein VP3 comprising an E500R amino acid substitution mutation;
wherein the AAV is AAV9 or a variant thereof and the amino acid substitution mutations are numbered relative to the AAV9 major capsid protein VP1 (SEQ ID NO: 1);
(a) a major capsid protein VP1 comprising T592D and A593E amino acid substitution mutations;
(b) a minor capsid protein VP2 comprising T592D and A593E amino acid substitution mutations; and/or
(c) a minor capsid protein VP3 comprising T592D and A593E amino acid substitution mutations;
wherein the AAV is AAV2 or a variant thereof and the amino acid substitution mutations are numbered relative to the AAV2 major capsid protein VP1 (SEQ ID NO: 2); or
(IV)
(a) a major capsid protein VP1 comprising I451Y, S454I, and P504I amino acid substitution mutations;
(b) a minor capsid protein VP2 comprising I451Y, S454I, and P504I amino acid substitution mutations; and/or
(c) a minor capsid protein VP3 comprising I451Y, S454I, and P504I amino acid substitution mutations;
wherein the AAV is AAV9 or a variant thereof and the amino acid substitution mutations are numbered relative to the AAV9 major capsid protein VP1 (SEQ ID NO: 1).

36-39. (canceled)

40. The AAV of claim 35, wherein:
(a) the major capsid protein VP1 comprises A273N, Q387K, A472H, and E500R amino acid substitution mutations;
(b) the minor capsid protein VP2 comprises A273N, Q387K, A472H, and E500R amino acid substitution mutations; and/or
(c) the minor capsid protein VP3 comprises A273N, Q387K, A472H, and E500R amino acid substitution mutations;
wherein the amino acid substitution mutations are numbered relative to the AAV9 major capsid protein VP1 (SEQ ID NO: 1).

41-101. (canceled)

102. The method of claim 1, wherein:
(a) the major capsid protein VP1 comprises I451Y, S454I, and P504I amino acid substitution mutations and further comprises an E500P or E500R amino acid substitution mutation;
(b) the minor capsid protein VP2 comprises I451Y, S454I, and P504I amino acid substitution mutations and further comprises an E500P or E500R amino acid substitution mutation; and/or
(c) the minor capsid protein VP3 comprises I451Y, S454I, and P504I amino acid substitution mutations and further comprises an E500P or E500R amino acid substitution mutation;
wherein the AAV is AAV9 or a variant thereof and the amino acid substitution mutations are numbered relative to the AAV9 major capsid protein VP1 (SEQ ID NO: 1).

103-131. (canceled)

132. A method for determining the affinity of an AAV capsid for a query protein, the method comprising:
(a) providing a virus-like particle (VLP) having a capsid consisting essentially of the VP3 subunit of the AAV capsid;
(b) contacting the VLP of step (a) with the query protein under conditions permitting the binding of the query protein and the VLP; and
(c) quantifying the strength of the interaction between the query protein and the VLP, thereby determining the affinity of the AAV capsid for the query protein.

133-142. (canceled)


SEQUENCE LISTING (19 sequences)

SEQ ID NO: 1 - protein, 736 aa, Adeno-associated virus serotype 9 (AAV9)
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 735 aa, Adeno-associated virus serotype 2 (AAV2) [sequence omitted]

SEQ ID NO: 3 - protein, 1049 aa, Homo sapiens
MEKRLGVKPNPASWILSGYYWQTSAKWLRSLYLFYTCFCFSVLWLSTDASESRCQQGKTQFGVGLRSGGENHLWLLEGTPSLQSCWAACCQDSACHVFWWLEGMCIQADCSRPQSCRAFRTHSSNSMLVFLKKFQTADDLGFLPEDDVPHLLGLGWNWASWRQSPPRAALRPAVSSSDQQSLIRKLQKRGSPSDVVTPIVTQHSKVNDSNELGGLTTSGSAEVHKAITISSPLTTDLTAELSGGPKNVSVQPEISEGLATTPSTQQVKSSEKTQIAVPQPVAPSYSYATPTPQASFQSTSAPYPVIKELVVSAGESVQITLPKNEVQLNAYVLQEPPKGETYTYDWQLITHPRDYSGEMEGKHSQILKLSKLTPGLYEFKVIVEGQNAHGEGYVNVTVKPEPRKNRPPIAIVSPQFQEISLPTTSTVIDGSQSTDDDKIVQYHWEELKGPLREEKISEDTAILKLSKLVPGNYTFSLTVVDSDGATNSTTANLTVNKAVDYPPVANAGPNQVITLPQNSITLFGNQSTDDHGITSYEWSLSPSSKGKVVEMQGVRTPTLQLSAMQEGDYTYQLTVTDTIGQQATAQVTVIVQPENNKPPQADAGPDKELTLPVDSTTLDGSKSSDDQKIISYLWEKTQGPDGVQLENANSSVATVTGLQVGTYVFTLTVKDERNLQSQSSVNVIVKEEINKPPIAKITGNVVITLPTSTAELDGSKSSDDKGIVSYLWTRDEGSPAAGEVLNHSDHHPILFLSNLVEGTYTFHLKVTDAKGESDTDRTTVEVKPDPRKNNLVEIILDINVSQLTERLKGMFIRQIGVLLGVLDSDIIVQKIQPYTEQSTKMVFFVQNEPPHQIFKGHEVAAMLKSELRKQKADFLIFRALEVNTVTCQLNCSDHGHCDSFTKRCICDPFWMENFIKVQLRDGDSNCEWSVLYVIIATFVIVVALGILSWTVICCCKRQKGKPKRKSKYKILDATDQESLELKPTSRAGIKQKGLLLSSSLMHSESELDSDDAIFTWPDREKGKLLHGQNGSVPNGQTPLKARSPREEIL

SEQ ID NO: 4 - protein, 4 aa, Adeno-associated virus serotype 9 (AAV9)
GCCT

SEQ ID NO: 5 - protein, 4 aa, Adeno-associated virus serotype 9 (AAV9)
AATT

SEQ ID NO: 6 - protein, 4 aa, Adeno-associated virus serotype 9 (AAV9)
AACT

SEQ ID NO: 7 - protein, 101 aa, Adeno-associated virus serotype 2 (AAV2)
LETQTQYLTPSLSDSHQQPPLVWELIRWLQAVAHQWQTITRAPTEWVIPREIGIAIPHGWATESSPPAPEPGPCPPTTTTSTNKFPANQEPRTTITTLATA

SEQ ID NO: 8 - protein, 101 aa, Adeno-associated virus serotype 13 (AAV13)
LATQSQSQTLNHSDNLPQPPLVWDLLQWLQAVAHQWQTITRVPMEWVIPQEIGIAIPNGWATESSPPAPAPGPCPPTTITSTSKSPANQEPPTTTTTLATA

SEQ ID NO: 9 - protein, 101 aa, Adeno-associated virus serotype 3b (AAV3b)
LATQSQSQTLNLSENHQQPPQVWDLIQWLQAVAHQWQTITRVPMEWVIPQEIGIAIPNGWATESSPPAPEPGPCPLTTTISTSKSPANQELQTTTTTLATA

SEQ ID NO: 10 - protein, 103 aa, Adeno-associated virus serotype 8 (AAV8)
LATQSQFQTLNLSENLQQRPLVWDLIQWLQAVAHQWQTITKAPTEWVVPREIGIAIPHGWATESSPPAPEPGPCPPTTTTSTSKSPTGHREEPPTTTPTSATA

SEQ ID NO: 11 - protein, 102 aa, Adeno-associated virus serotype 6 (AAV6)
LATQSQSPTHNLSENLQQPPLLWDLLQWLQAVAHQWQTITKAPTEWVMPQEIGIAIPHGWATESSPPAPEHGPCPPITTTSTSKSPVLQRGPATTTTTSATA

SEQ ID NO: 12 - protein, 102 aa, Adeno-associated virus serotype 1 (AAV1)
LATQSQSPIHNLSENLQQPPLLWDLLQWLQAVAHQWQTITKAPTEWVMPQEIGIAIPHGWATESSPPAPAPGPCPPTITTSTSKSPVLQRGPATTTTTSATA

SEQ ID NO: 13 - protein, 103 aa, Adeno-associated virus serotype 10 (AAV10)
LASQSQSPTLNQSENHQQAPLVWDLVQWLQAVALQWQTITKAPTEWVVPQEIGIAIPHGWATESSPPAPEPGPCPPTTTTSTSKSPTGHREEAPTTTPTSATA

SEQ ID NO: 14 - protein, 102 aa, Adeno-associated virus serotype 7 (AAV7)
LATQSQSPTLNLSENLQQRPLVWDLVQWLQAVAHQWQTITKVPTEWVMPQEIGIAIPHGWATESLPPAPEPGPCPPTTTTSTSKSPVKLQVVPTTTPTSATA

SEQ ID NO: 15 - protein, 103 aa, Adeno-associated virus serotype 9 (AAV9)
LATQSQSQTLNQSENLPQPPQVWDLLQWLQVVAHQWQTITKVPMEWVVPREIGIAIPNGWGTESSPPAPEPGPCPPTTITSTSKSPTAHLEDLQMTTPTSATA

SEQ ID NO: 16 - protein, 92 aa, Adeno-associated virus serotype 4 (AAV4)
LEQATDPLRDQLPEPCLMTVRCVQQLAELQSRADKVPMEWVMPRVIGIAIPPGLRATSRPPAPEPGSCPPTTTTSTSDSERACSPTPTTDSP

SEQ ID NO: 17 - protein, 92 aa, Adeno-associated virus serotype 11 (AAV11)
LEPETDPLKDQIPAPCLQTLKCVQHRAEMLSMRDKVPMEWVMPRVIGIAIPPGLRARSQQPRPEPGSCPPTTTTCTCVSEQHQAATPTTDSP

SEQ ID NO: 18 - protein, 92 aa, Adeno-associated virus serotype 12 (AAV12)
LEQETDPLRDHLPEKCLMMLRCVRRQAEMLSRRDKVPMEWVMPPVIGIAIPPGQRAESPPPAPEPGSYPRTTTTCTCESEQRPTATPTTDSP

SEQ ID NO: 19 - protein, 98 aa, Adeno-associated virus serotype 5 (AAV5)
LDPADPSSCKSQPNQPQVWELIQCLREVAAHWATITKVPMEWAMPREIGIAIPRGWGTESSPSPPEPGCCPATTTTSTERSKAAPSTEATPTPTLDTA